(19) United States
(12) Patent Application Publication
(10) Pub. No.: US 2020/0190574 A1
(43) Pub. Date: Jun. 18, 2020

(54) RNA-STITCH SEQUENCING: AN ASSAY FOR DIRECT MAPPING OF RNA : RNA INTERACTIONS IN CELLS

(71) Applicant: The Regents of the University of California, Oakland, CA (US)

(72) Inventors: Sheng Zhong, San Diego, CA (US); Tri Cong Nguyen, San Diego, CA (US)

(21) Appl. No.: 15/462,680

(22) Filed: Mar. 17, 2017

Related U.S. Application Data
(63) Continuation of application No. PCT/US2015/051075, filed on Sep. 18, 2015.
(60) Provisional application No. 62/053,615, filed on Sep. 22, 2014.

(51) Int. Cl. C12Q 1/68; G06F 19/18; G06F 19/24

(52) U.S. Cl. CPC C12Q 1/6874; C12Q 1/6876; G06F 19/18; C12Q 2600/178; G06F 19/24; C12Q 2600/136; C12Q 1/6806

(57) ABSTRACT

Methods and compositions for generating chimeric RNAs comprising RNAs which interact with one another in a cell are provided. In some embodiments, the chimeric RNAs can be used to identify at least 100, at least 500, at least 1000 or more than 1000 RNA-RNA interactions in the cell.

### CROSS REFERENCE TO RELATED APPLICATIONS

The present application is a continuation of PCT/US2015/051075 filed Sep. 18, 2015, which claims the benefit of priority to U.S. Provisional Patent Application No. 62/053,615, filed on Sep. 22, 2014. The entire disclosures of the aforementioned applications are expressly incorporated herein by reference in their entirety.

### STATEMENT REGARDING FEDERALLY SPONSORED R&D

This invention was made with government support under grant number NIH DP2-OD007417 awarded by the National Institute of Health. The government has certain rights in the invention.

### REFERENCE TO SEQUENCE LISTING, TABLE, OR COMPUTER PROGRAM LISTING

The present application is being filed along with a Sequence Listing in electronic format. The Sequence Listing is provided as a file entitled UCSD089.001C1 Substitute .TXT, created Jun. 8, 2017, which is 11 Kb in size. The information in the electronic format of the Sequence Listing is incorporated herein by reference in its entirety.

### BACKGROUND OF THE INVENTION

### Field of the Invention

Methods and compositions for identifying RNAs which interact with one another in a cell are provided.

### Description of the Related Art

Currently, there are no efficient methods that can directly assay substantially all RNA-RNA interactions in a cell type at once. There are two kinds of methods which exist to partially achieve this goal, both with weaknesses. Technologies like HITS-CLIP and CLASH can detect targets of many miRNAs. However, both methods concentrate on miRNAs, which only comprise a small portion of RNAs. Thus, these technologies are not able to reveal the majority of RNA-RNA interactions. Furthermore, each technology has additional weaknesses. For example, direct pairing of a miRNA to their target mRNAs cannot be directly deduced from HITS-CLIP. In other words, HITS-CLIP does not directly inform which miRNA regulates which mRNAs (no one-to-one information).

A recent method called CLASH (cross-linking, ligation, and sequencing of hybrids) could allow direct observation of miRNA-target pairs. However, the number of interactions is still small as compared to the number of sequencing reads: only 2% of sequenced reads are chimeric, 98% are still single reads. This requires much deeper sequencing coverage or preparation of multiple samples to obtain enough coverage of miRNA-mRNA interactions.

### SUMMARY OF THE INVENTION

Some embodiments of the present invention are provided in the following numbered paragraphs:

1. A method for generating chimeric RNAs comprising RNAs which interact with one another in a cell comprising cross-linking RNA to protein and ligating RNAs cross-linked to the same protein molecule together to form a chimeric RNA.

2. The method of Paragraph 1, wherein said cross-linking of RNA to protein is performed on an intact cell or in a cell lysate.

3. The method of any one of Paragraphs 1 or 2 wherein said cross-linking comprises UV cross-linking.

4. The method of any one of Paragraphs 1-3, further comprising associating said protein with an agent which facilitates immobilization of said protein on a surface.

5. The method of Paragraph 4, wherein said agent which facilitates immobilization comprises biotin.

6. The method of any one of Paragraphs 1-5, further comprising fragmenting said RNAs cross-linked to the same protein molecule.

7. The method of Paragraph 6, wherein said fragmenting comprises contacting said RNAs cross-linked to the same protein molecule with an RNAse under conditions which facilitate partial digestion of said RNAs.

8. The method of any one of Paragraphs 1-7, further comprising linking said RNAs cross-linked to the same protein molecule to an agent which facilitates recovery of said RNAs.

9. The method of Paragraph 8, wherein said linking comprises ligating the ends of said RNAs to said agent.

10. The method of Paragraph 9, wherein said agent facilitates recovery of said RNAs comprises a nucleic acid.

11. The method of Paragraph 10, wherein said nucleic acid comprises a nucleic acid having biotin thereon.

12. The method of Paragraph 11, wherein said linking of said nucleic acid having biotin thereon to said ends of said RNAs comprises ligating said nucleic acid having biotin thereon to the 5′ ends of said RNAs prior to ligating said RNAs cross-linked to the same protein molecule together to form a chimeric RNA.

13. The method of Paragraph 12, further comprising removing said biotin from the 5′ region of said chimeric RNA.

14. The method of any one of Paragraphs 1-13, further comprising recovering said chimeric RNAs.

15. The method of any one of Paragraphs 1-14, further comprising fragmenting said chimeric RNAs.

16. The method of any one of Paragraphs 1-15, wherein said fragmenting of said chimeric RNAs comprises contacting said chimeric RNAs with an RNAse under conditions which facilitate partial digestion of said RNAs.

17. The method of any one of Paragraphs 1-16, further comprising reverse transcribing said chimeric RNAs to generate a chimeric cDNA.

18. The method of any one of Paragraphs 1-17, further comprising determining at least a portion of the sequences in said chimeric RNAs or chimeric cDNAs which originate from each of the RNAs in said chimeric RNAs or chimeric cDNAs.

19. The method of any one of Paragraphs 1-17, further comprising identifying the RNAs present in said chimeric RNAs, thereby identifying RNAs which interact with one another in a cell.

20. The method of Paragraph 19, wherein at least 100, at least 500, at least 1000 or more than 1000 RNA-RNA interactions in the cell are identified.

21. The method of Paragraph 19, wherein substantially all of the RNAs which interact with one another in a cell are identified.

22. The method of Paragraph 21, wherein at least 70%, at least 80%, at least 90% or more than 90% of the direct RNA-RNA interactions in the cell are identified.

23. The method of any one of Paragraphs 19-22, wherein the identification of the RNAs which interact with one another in a cell comprises performing sequence reads on said chimeric RNAs using an automated sequencing device.

24. The method of Paragraph 23, wherein said identification of the RNAs which interact with one another in a cell comprises identifying the chimeric sequences from all the sequence reads.

25. The method of any one of Paragraphs 19-24, further comprising transforming the chimeric RNAs into annotated RNA clusters using a computer.

26. The method of Paragraph 25, further comprising identifying direct interactions among said RNA clusters using a statistical test performed by a computer.

27. An isolated complex comprising a chimeric RNA cross-linked to a protein, wherein said chimeric RNA comprises RNAs which interact with one another in a cell.

28. A method for identifying a candidate therapeutic agent comprising:
identifying RNAs which interact with one another in a cell using the method of any one of Paragraphs 1-26; and
evaluating the ability of an agent to reduce or increase the interaction of said RNAs, wherein said agent is a candidate therapeutic agent if said agent is able to reduce or increase said interaction of said RNAs.

29. The method of Paragraph 28, wherein said agent comprises a nucleic acid.

30. The method of Paragraph 28, wherein said agent comprises a chemical compound.

31. A method of making a pharmaceutical comprising formulating an agent identified using the method of any one of Paragraphs 28-30 in a pharmaceutically acceptable carrier.

32. A pharmaceutical made using the method of Paragraph 31.

33. A method for generating chimeric RNAs comprising RNAs which interact with one another in a cell comprising cross-linking RNA to protein intermediates and/or a protein complex and ligating RNAs cross-linked to protein intermediates and/or the protein complex together to form a chimeric RNA, and wherein the protein complex comprises two or more interacting proteins.

34. The method of Paragraph 33, wherein said cross-linking of RNA to the protein intermediates and/or the protein complex is performed on an intact cell or in a cell lysate.

35. The method of any one of Paragraphs 33 or 34 wherein said cross-linking comprises UV cross-linking.

36. The method of any one of Paragraphs 33-35, further comprising associating said protein intermediates and/or the protein complex with an agent which facilitates immobilization of said protein intermediates and/or the protein complex on a surface.

37. The method of Paragraph 36, wherein said agent which facilitates immobilization comprises biotin.

38. The method of any one of Paragraph s 33-37, further comprising fragmenting said RNAs cross-linked to the at least one protein molecule.

39. The method of Paragraph 38, wherein said fragmenting comprises contacting said RNAs cross-linked to the protein intermediates and/or the protein complex with an RNAse under conditions which facilitate partial digestion of said RNAs.

40. The method of any one of Paragraph s 33-39, further comprising linking said RNAs cross-linked to the protein intermediates and/or the protein complex to an agent which facilitates recovery of said RNAs.

41. The method of Paragraph 40, wherein said linking comprises ligating the ends of said RNAs to said agent.

42. The method of Paragraph 41, wherein said agent which facilitates recovery of said RNAs comprises a nucleic acid.

43. The method of Paragraph 42, wherein said nucleic acid comprises a nucleic acid having biotin thereon.

44. The method of Paragraph 43, wherein said linking of said nucleic acid having biotin thereon to said ends of said RNAs comprises ligating said nucleic acid having biotin thereon to the 5′ ends of said RNAs prior to ligating said RNAs cross-linked to the protein intermediates and/or the protein complex together to form a chimeric RNA.

45. The method of Paragraph 44, further comprising removing said biotin from the 5′ region of said chimeric RNA.

46. The method of any one of Paragraph s 33-45, further comprising recovering said chimeric RNAs.

47. The method of any one of Paragraph s 33-46, further comprising fragmenting said chimeric RNAs.

48. The method of any one of Paragraph s 33-47, wherein said fragmenting of said chimeric RNAs comprises contacting said chimeric RNAs with an RNAse under conditions which facilitate partial digestion of said RNAs.

49. The method of any one of Paragraph s 33-48, further comprising reverse transcribing said chimeric RNAs to generate a chimeric cDNA.

50. The method of any one of Paragraph s 33-49, further comprising determining at least a portion of the sequences in said chimeric RNAs or chimeric cDNAs which originate from each of the RNAs in said chimeric RNAs or chimeric cDNAs.

51. The method of any one of Paragraph s 33-49, further comprising identifying the RNAs present in said chimeric RNAs, thereby identifying RNAs which interact with one another in a cell.

52. The method of Paragraph 51, wherein at least 100, at least 500, at least 1000 or more than 1000 RNA-RNA interactions in the cell are identified.

53. The method of Paragraph 51, wherein substantially all of the RNAs which interact with one another in a cell are identified.

54. The method of Paragraph 53, wherein at least 70%, at least 80%, at least 90% or more than 90% of the direct RNA-RNA interactions in the cell are identified.

55. The method of any one of Paragraph s 51-54, wherein the identification of the RNAs which interact with one another in a cell comprises performing sequence reads on said chimeric RNAs using an automated sequencing device.

56. The method of Paragraph 55, wherein said identification of the RNAs which interact with one another in a cell comprises identifying the chimeric sequences from all the sequence reads.

57. The method of any one of Paragraph s 51-56, further comprising transforming the chimeric RNAs into annotated RNA clusters using a computer.

58. The method of Paragraph 57, further comprising identifying direct interactions among said RNA clusters using a statistical test performed by a computer.

59. The method of any one of Paragraph s 33-58, wherein said RNAs which interact with each other in the cell are cross-linked to different proteins in said protein intermediate or protein complex.

60. An isolated complex comprising a chimeric RNA cross-linked to protein intermediates and/or a protein complex, wherein said chimeric RNA comprises RNAs which interact with one another in a cell, wherein the protein complex comprises two or more interacting proteins.

61. The isolated complex of Paragraph 59, wherein said chimeric RNA comprises RNAs which are cross-linked to different proteins in said protein intermediate or protein complex

### BRIEF DESCRIPTION OF THE DRAWINGS

FIGS. 1A to 1D. RNA Hi-C. (FIG. 1A) The major experimental steps: 1. cross-linking RNAs to proteins, 2. RNA fragmentation and protein biotinylation (the ball represents the biotin), 3. immobilization, 4. ligation of a biotinylated RNA linker (The ball on the strand is the biotin on the linker) 5. proximity ligation under an extremely dilute condition, 6. RNA purification and reverse transcription, 7. biotin pull-down. 8. construction of sequencing library. Shown in the chimeric RNA schematic is the desired chimeric products which have the P5 specific primer, the barcode between the Pr specific primer and the RNA1, the Linker specific reverse primer between the RNA1 and RNA2, followed by the P7 region. In the incomplete product shown, the P5 region is adjacent to the barcode, the barcode is between the P5 region and the linker, the RNA2 region and then the P7 region. (FIG. 1B) PCR validation of RNA1-Linker-RNA2 chimeras, which were expected to be above 91 bp from the P5 sequencing primer to the linker and above 200 bp from P5 to P7 sequencing primers. The failure to include RNA1 would create 91 bp products from P5 to the linker. The failure to include RNA2 would create similar sized products from P5 to the linker and from P5 to P7. The PCR primers are marked on top of each lane. The size distribution of the sequencing libraries was also assessed by Bioanalyzer. As shown in the desired chimeric products from left to right is the P5 specific fwd primer, the barcode, the RNA1, the linker (complimentary to the linker specific primer, the RNA2, and P7. As shown in the incomplete product, is the P5, the barcode, linker, RNA2 and P7. (FIG. 1C) RNA Hi-C data mapped to the genome. Ligation of Trim25 and Snora1 RNAs was experimentally supported by 46 pair-end reads in ES-1 and ES-2 libraries. Ago CLIP-seq: AGO HITS-CLIP of mouse ES cells (GEO: GSM622570). Small RNA-seq: sequencing of small RNAs with a 3′ hydroxyl group resulting from enzymatic cleavage (GEO: GSM945907). (FIG. 1D) Large modules of the RNA interactome. Small modules involving 4 or less interacting RNAs were not shown. The interactions involving snoRNAs, snRNAs, and tRNAs were not shown. The large majority of the sequences in the list are mRNA, the rest are pseudogenes (FP130=ps3, Gm16580, Gm12715, Gm13226, Rp128-ps3, Fp128-ps1, Rps16-ps2, Gm4707, Gm13340, Gm13408, Gm15590, Gr12, Gm11400, Gm17087, Gm15725, Gm12346, Gm11478), lincRNA (Gm16869, Malat1, Snhg7, Gm16702, 4930417H01Rik), miRNA (Mir5100, Mir692-1, Mir692-2b, Ac117657, Mir5099) and antisense RNA (Gm15444).

FIGS. 2A to 2E. RNA interaction sites. (FIG. 2A) Multiple RNA Hi-C reads, representative of different interactions (dashed lines), overlapped on specific regions of the Eef1a1 gene. (FIG. 2B) Finding interaction sites by the “peaks” of overlapping reads. Peak 1 and 2 are the RNA2, Peak 3 and 4 are RNA2. (FIG. 2C) Distribution of interaction sites in different types of RNA genes and transposons. (FIG. 2D) The distribution of binding energies (ΔG, kcal/mol) between the interaction sites of two RNAs (light grey, left), and between randomly shuffled bases (white, right). P-values from Wilcoxon rank test are marked at the bottom of each panel. (FIG. 2E) Conservation levels, measured by average PhyloP scores peaked at the junction (black bar, position 0 on the x axis) of the ligated RNA fragments. Control: conservation levels of randomly selected genomic regions. As shown, in the graph, the data on the left represents RNA1 and the data on the right represents RNA2.

FIGS. 3A to 3F. RNA structure. (FIG. 3A) Schematic depiction of resolving the proximal sites of an RNA. Pointer arrow on the schematic of the nucleic acid: RNase I cutting site. (FIG. 3B) The “cut and ligated” products mapped to Snora73. Vertical color bar: a cluster of read pairs supporting a pair of proximity sites. The numbers on the proximity sites correspond to the numbers on the sequence in FIGS. 3E and 3F. (FIG. 3C) Density of RNase I cuts. The numbers on the proximity sites correspond to the numbers on the sequence in FIGS. 3E and 3F. (FIG. 3D) Heatmap of the ligation frequencies between any two positions of the RNA. Each colored circle corresponds to a vertical color bar in FIG. 3A, and represents a pair of proximal sites. (FIG. 3E) Footprint of single stranded regions and inferred proximal sites on the accepted secondary structure. (FIG. 3F) A pair of inferred proximal sites, that was not supported by sequenced-based secondary structure, are physically close in vivo, due to protein assisted RNA folding.

FIG. 4. Shown is a step by step sequencing based technology to map RNA-RNA interactions.

FIGS. 5A and 5B. Workflow for computational part. (FIG. 5A) A flowchart for identification of the chimeric RNA sequences. As shown in the inset box of the primary sequences are sequences of “No linker”, “Linker Only”, “Back Only,” “Front Only,” and “Paired.” As shown the No linker sequences have: 1) 5′Index, 2) 5′ Index, Part 1, and Part 2, 3) 5′ Index, Part 1, and 3) 5′Index and Part2. As shown, the Linker only sequence has a 5′Index and Part 2. As shown the BackOnly has 5′Index, Linkers, and Part 2. As shown the FrontOnly has a 5′Index and Linkers. As shown the Paired has a 5′Index, Part 1, Linkers and Part2. (FIG. 5B) Illustration of how to Identify RNA-RNA interactions that are supported by large numbers of chimeric RNAs. As shown in the top panel are segments in R1 and in the lower panel, segments in R2. As shown in the graph, they are paired in chimeric RNA.

FIGS. 6A to 6D. Preliminary results. (FIG. 6A) Size distribution of the library of chimeric cDNA. Note that 128 bp are primer sequences. (FIG. 6B) Proportions of interactions between different types of RNAs. (FIG. 6C) Eighteen ligated RNA pairs were mapped to SNORA1 and Trim25. The mapped loci coincided with Ago CLIP-seq data (GSM622570). (FIG. 6D) The reverse correlation of SNORA1 and Trim25 during a guided differentiation process. As shown, Trim25 decreases from about 35 RNA-seq RPKM to about 5 at day 4, while SNORA1 increases from Day 0 to Day 6.

FIGS. 7A to 7B. A circularization strategy for construction of sequencing libraries. This figure elaborates Step 8 of the RNA Hi-C procedure. (FIG. 7A) A reverse transcription (RT) adaptor was attached to the 3′ end of the RNAs. This RT adaptor was complementary to a fraction of a RT primer, which also contained an adaptor for the P5 sequencing primer, a 10 nt barcode, and a BamHI restriction site. After circularization, a DNA oligo containing the BamHI site was hybridized to the RT primer region, providing a double stranded substrate for BamHI digestion. Linearized ss-cDNAs were amplified by truncated PCR primers DP5 and DP3 to obtain ˜100 ng of ds-cDNAs, which were then denatured and reannealed. Duplex-specific nuclease (DSN) was used to deplete cDNAs that were originated from rRNAs. DSN selectively removes the ds-cDNAs that were formed earlier during the reannealing process. The cDNAs originated from rRNAs should be more abundant and therefore reanneal faster than the other cDNAs. The DSN-treated products were PCR-amplified again by Illumina PCR primers PE 1.0 and 2.0 to generate libraries suitable for sequencing. DSN based rRNA removal was applied to ES-1. ES-2 was subjected to an antibody based rRNA removal strategy that is not depicted in this figure. As shown at the end is the product of P5, the barcode, RNA1, the Adaptor, RNA2, and P7 (FIG. 7B).

FIG. 8. Description of the RNA Hi-C samples. The “total # of read pairs” is the number of pair-end sequencing reads for each sample. The “# of non-duplicate read pairs in the form of RNA1-Linker-RNA2” is the number of the pair-end reads in the output of Step 4, parsing the chimeric cDNAs, of the bioinformatic pipeline.

FIGS. 9A to 9E. Optimizing RNase I concentration for the first fragmentation. RNAs were purified from RNaseI-treated ES cell lysate by adding equal volume of 2× Proteinase K buffer (100 mM Tris-HCl pH 7.5, 100 mM NaCl, 2% SDS, 20 mM EDTA) and 1:5 volume of 20 mg/ml Proteinase K (NEB) and incubating at 55° C. for 2 hours before phenol:chloroform treatment and ethanol precipitation. RNase I quantity per ml of cell lysate were: 0 U (Sample 1, FIG. 9A), 2.5 U (Sample 2 (FIG. 9B)), 3.3 U (Sample 3, FIG. 9C), 5 U (Sample 4, FIG. 9D), and 12.5 (Sample 5, FIG. 9E). The concentration of 5.0 U RNase I/ml lysate that produced 500-1000 nt RNA fragments (Sample 4) was chosen for RNA Hi-C Step 2.

FIG. 10. Testing the efficiency of linker ligation on beads. Immobilized RNAs were digested with RNase I and then ligated with the biotin-labelled RNA linkers (1). After ligation and proteinase K digestion to remove the proteins, RNAs were purified and quantified (1.3 μg) (2). The purified RNAs were then subjected to streptavidin-biotin pulldown to select for RNAs ligated to the biotin-labelled linker (3). After washing and eluting RNAs that were bound to streptavidin beads and ethanol precipitated, 0.22 μg of RNA was collected. In parallel, the biotin-labeled RNA linkers were subjected to the same streptavidin-biotin pulldown, elution and ethanol precipitation (4). Assuming that the efficiencies of biotin pulldown, RNA elution and ethanol precipitation in Steps 3 and 4 were the same, about 19.6% (1.96 μg/10.0 μg), it is estimated that the ligation efficiency (0.22 μg/19.6%)/1.3 μg=86%.

FIG. 11. RNA size distributions at different steps of the RNA Hi-C procedure. Only the ES-indirect and the MEF samples had sufficient intermediate products left for this retrospective analysis. Size distributions of RNAs in the lysates of MEF (Lane 1) and ES-indirect (Lane 2) before being tethered onto streptavidin beads, in the supernatant after immobilization (Lanes 3 and 4), and immobilized on beads after proximity ligation (ES-indirect: Lane 5, MEF: Lane 6). RNA was denatured in 2×RNA loading dye (NEB) at 70° C. for 5 minutes, run on 1.5% Native Agarose gel and stained with SYBR Gold (Invitrogen).

FIG. 12. Optimization of the number of PCR cycles for construction of sequencing library. In Step 8 of the RNA Hi-C procedure, single-stranded cDNAs of the ES-1 sample were pre-amplified with 12 cycles of PCR using a truncated form of Illumina PCR sequencing primers (DP5, DP3). The PCR products were purified with 1.8×SPRISelect beads, which produced 86 ng of double-stranded DNAs before the depletion of the cDNA synthesized from rRNA by duplex-specific nuclease. One μl aliquots from a total of 22 μl of rRNA-depleted double-stranded cDNAs were amplified with various PCR cycle numbers (12, 15, 18) using NEBNext High-Fidelity 2×PCR Master Mix (NEB) and Illumina PE Primer 1.0 and 2.0. The PCR products were assayed on 6% TBE PAGE gel and stained with SYBR Gold (Invitrogen). Based on the gel result, 18 μl of original rRNA depleted double-stranded DNAs were then amplified with 11 cycles of PCR to generate the sequencing library.

FIGS. 13A to 13C. Comparison of RNA Hi-C libraries. (FIGS. 13A-13B) The read fragment at the 5′ end (RNA1) and the 3′ end (RNA2) of the linker were separately analyzed as two RNA-seq experiments. Scatter plots of the read count distribution (FPKM) of all known RNAs between ES-1 and ES-2 samples at log scale. R: Pearson correlation. S: Spearman correlation. (FIG. 13 C) Hierarchical clustering of FPKMs of each sample.

FIG. 14. The online documentation for RNA-HiC-tools. This online resource (http://systemsbio.ucsd.edu/RNA-Hi-C) includes detailed descriptions of analysis and visualization tools, usage examples, sample output files and figures. Some tools are also provided as application programming interfaces (APIs).

FIGS. 15A to 15E. The computational pipeline for analysis of RNA Hi-C data. (FIG. 15A) PCR duplicates were removed from the pair-end sequencing reads (Step 1). Multiplexed samples were separated based on the 4 nt experimental barcodes (‘XXXX’, Step 2). ‘N’: a nucleotide of the random barcode. ‘X’: a nucleotide of the experimental barcode. (FIG. 15B) Each pair of forward (Read1) and reverse (Read2) reads were used to recover a cDNA in the input sequencing library, if possible. (FIG. 15C) The recovered cDNA were categorized based on the configuration of the RNA fragments and the linker sequence (Step 4). The RNA1-Linker-RNA2 type of cDNAs were provided as the output. (FIG. 15D) The RNA1 and the RNA2 parts were separately mapped to the genome. The output was the cDNAs where both RNA1 and RNA2 were uniquely mapped to the genome. (FIG. 15E) RNA-RNA interactions were identified based on association tests. As shown, Cluster 1 and Cluster 2 have the RNA1 and Cluster 3 and 4 have the RNA2.

FIGS. 16A-16C. Visualization capabilities of RNA-HiC-tools. (FIG. 16A-FIG. 16B) Detailed views of RNA interaction sites in intra-RNA (FIG. 16A) and inter-RNA (FIG. 16B) interactions. The two genomic regions containing the two interacting RNAs were plotted in parallel (FIG. 16B). Each RNA1-Linker-RNA2 type of chimeric RNA was plotted with the RNA1 and the RNA2 fragments mapped to the respective genomic regions, connected by an oblique line representing the linker. The blocks represent the “peaks” of overlapping RNA Hi-C reads, which were candidate RNA interaction sites. A semi-transparent polygon connecting two RNA interaction sites represents a strong interaction. (FIG. 16C) A global view of the RNA-RNA interactions. The read densities of the RNA1 and the RNA2 fragments were shown in the shaded areas, respectively, inside chromatin cytoband ideogram. Each identified RNA-RNA interaction was shown as a curve connecting the genomic loci of the two RNAs, and colored by the types of the interacting RNAs.

FIGS. 17A to 17D. snoRNAs with miRNA-like interactions. (FIG. 17A) Comparison of RNA Hi-C with smallRNA-seq (GSM945907) and AGO HITS-CLIP (GSM622570). The average FPKM of each type of RNA Hi-C identified interaction participating RNAs in smallRNA-seq and AGO HITS-CLIP is shown in log scale. The miRNAs and snoRNAs in RNA Hi-C identified interactions were enriched in both smallRNA-seq and AGO HITS-CLIP. As shown in FIG. 17A, the graph is represented such that the bars for representing the smallRNA-seq data is over the bars that represent the HITS-CLIP data. (FIG. 17B) Distribution of the correlations of gene expression between every pair of interacting snoRNA and mRNA. The interacting snoRNA-mRNA pairs bound by AGO (dark grey)(defined by AGO HITS-CLIP) were more negatively correlated than the pairs not bound by AGO (light grey) (p-value=4.18-5, Kolmogorov-Smirnov Test). As shown, an AGO-Bound peak shows up at about 0.075, 0.25, 0, −0.5 and −1 correlations. (FIG. 17C) Base pairing of the interacting RNAs as measured by hybridization energy. The snoRNA-mRNA pairs bound by AGO (intersected with AGO HITS-CLIP, left) exhibited stronger hybridization energies than those not bound by AGO (right) (p-value <2.2-16, Wilcoxon signed-rank test). All these interactions exhibited stronger hybridization energies than those with randomly shuffled sequences. As shown, the dark grey indicates the “Real” and the light represents “random.” (FIG. 17D) The snoRNAs interacted with the UTR regions of mRNAs were enriched in smallRNA-seq and AGO HITS-CLIP. The total number of interactions (y axis) between snoRNAs and mRNA coding regions (left) is decomposed into those detected in both smallRNA-seq and HITS-CLIP, in smallRNA-seq only, in HITS-CLIP only, and in neither datasets. The interactions between snoRNAs and mRNA UTRs were similarly decomposed (right). As shown in the left bar graph, the top portions are smallRNA and CLIP, followed by the CLIP data, small RNA, and “Neither.”

FIG. 18. Comparisons between RNA Hi-C and smallRNA-seq and AGO HITS-CLIP. The percentages of RNA Hi-C identified interactions that intersected with smallRNA-seq, AGO HITS-CLIP, and both. The RNA Hi-C interactions were categorized by the types of participating RNAs, and the categories were ranked by the overlap with HITS-CLIP. misc_RNA: miscellaneous RNA, including RNase_MRP, 7SK RNA and others. Novel: unannotated RNA. As shown the data is divvied from the top to bottom as the “overlap with both”, the “overlap with smallRNA-seq” data, and the “overlap with HITS-CLIP” data.

FIGS. 19A to 19C. Interaction between enzymatically processed SNORA14 and Mcl1 mRNA. (FIG. 19A) The RNA Hi-C identified interaction site on SNORA14 intersected with small RNA-seq, suggesting the SNORA14 RNA was enzymatically processed into a shorter form (highlighted region on the peak, 2nd row). This enzymatically processed small RNA corresponded to the end of the SNORA14 hairpin (highlighted region on the secondary structure), as well as the antisense to 3′ UTR of Mcl1 (highlighted region in (FIG. 19B) above the SNOARA14 sequence)). (FIG. 19C) Expression levels of the small RNA processed from SNORA14 RNA and Mcl1 mRNA during the differentiation of ES cells to endomesoderm cells. As shown, Mcl1 decreases from Day 0 to Day 6, while SNORA14 increases from Day 0 to Day 6.

FIGS. 20A to 20D. Distributions of read counts and FDRs and relationships with gene expression. (FIG. 20A) Distribution of the number of read pairs mapped to every pair of RNAs. (FIG. 20B) Distribution of FDRs of every RNA pair from Fisher's Exact Test. (FIG. 20C) Scatter plot of the number of RNA Hi-C reads mapped to each RNA (y axis) and FPKM (x axis). (FIG. 20D) Scatter plot of the smallest FDR (in minus log) associated with the interactions of each RNA and the FPKM of this RNA. The FPKM values were obtained by mapping raw reads from mouse ENCODE dataset ENCSR000CWC (paired-end RNA-Seq from E14 mouse ES cells) [1] with bowtie2-2.2.4 against mm9, followed by processing with cufflink 2.2.1. All the genes with unique Ensembl IDs that were found in both ENCSR000CWC data and RNA-Hi-C mouse ES cell data are included in FIG. 20C and FIG. 20D.

FIG. 21. Distribution of the 46,780 identified RNA-RNA interactions among different types of RNAs. rRNAs were experimentally (experimental Step 6.2) and bioinformatically (analysis Step 6) removed from the analysis.

FIGS. 22A and 22B. Degree distribution of the RNA-RNA interaction network. The number of nodes (RNAs) was inversely proportional to their degrees (number of interactions) in the log scale (FIG. 22A), characteristic of scale-free networks. This property was not changed after removing snRNAs, snoRNAs and tRNAs from the network (FIG. 22B).

FIG. 23. Distribution of interaction sites in different types of genes and transposons. Novel: unannotated genomic regions.

FIGS. 24A to 24F. Examples of base complementation between RNA Hi-C identified interacting RNAs. The types of interacting RNAs included mRNA-mRNA (FIG. 24A), lincRNA-mRNA (FIG. 24B), pseudogeneRNA-mRNA (FIG. 24C), mRNA-LTR (FIG. 24D), LINE-mRNA (FIG. 24E), mRNA-miRNA (FIG. 24F). LTR and LINE represent transposon transcripts. The curves on the left hand side of the sequences linking the 3′ end of the RNA to the second RNA represent linker positions. The number of ligated chimeric RNAs supporting each interaction are given in the brackets next to the curves. AG: hybridization energy. Shuffle: the average hybridization energy of randomly shuffled bases.

FIGS. 25A to 25C. Conservation levels of interacting RNAs. Interactions were categorized by RNA types. For each type of interactions, the conservation level was approximated by the average PhyloP scores of the genomic regions (1000 bp) centered at the RNA ligation junctions (position 0 on the x axis). The conservation levels of random genomic regions of the same lengths were plotted as controls. On the bottom of the graphs are representations of the RNA1 (right) and RNA2 (left) fragments of a RNA1-Linker-RNA2 chimeric RNA. Dashed line: the linker. As shown in FIG. 25A is the structure with mRNA, FIG. 25B with LINE, and FIG. 25C with the LTR.

FIG. 26. Comparison of the conservation levels. Conservation levels were quantified by the average PhyloP score per nucleotide of the interaction sites (y axis). To adjust for the difference of conservation of exons, introns, and UTRs, the interaction sites (bars on the left side of the paired bars) in annotated exons, introns, and UTRs (dubbed genomic features) were compared to 200,000 randomly sampled genomic sequences from the same genomic feature (bars on the right side of the paired bars). The sizes of the randomly sampled genomic sequences shared the same mean and variation as the sizes of interaction sites. P-values were calculated from one-sided two-sample t-test. **: p-value <10-12; *: p-value <10-6.

FIGS. 27A to 27D. Correlation of RNase I digestion density and single-stranded regions for various transcripts: Snora73 transcripts (FIG. 27A), Snora74a transcripts (FIG. 27B). Snora81 transcripts (FIG. 27C), and Snora94 transcripts (FIG. 27D). The frequency of digestion measured by the number of read fragments ending or starting at each position (y axis) was compared to known secondary structure (fRNAdb database v3.4) (x axis). Brackets on the x axis represent double-stranded regions. The total counts of read fragments ending or starting at each position in single-stranded (ss) and double-stranded (ds) are summarized on the right panels.

FIGS. 28A to 28C. Intramolecular ligations. (FIG. 28A) An intramolecular (self) ligation was generated by RNase I digestions of a transcript followed by a linker ligation and a proximity ligation. Therefore, the two RNA fragments on the two sides of the linker came from the same RNA molecule. These intramolecular ligation events were identified with stringent bioinformatic criteria, filtering out pair-end reads that could have been generated from a consecutive transcript. The pair-end reads that could only been generated by a cut-and-ligation process were used for RNA structure analysis. Lower panel: the distribution of intramolecular ligations among different RNA types. (FIG. 28B) The number of intramolecular ligations (y axis) versus the transcript length (x axis) by RNA types. Error bars: standard deviation of the mean. Shown is the lincRNA at less than 10 ligations per gene at a length of over 1000 nt, tRNA at less than 10 self-ligations per gene and a length of less than 100 nt, snoRNA at over 100 self-ligations per gene and a length of over 100 nt and snRNA at less than 100 self-ligations per gene and a length of over 100 nt. (FIG. 28C) The number (shaded bars) and the lengths (box plots) of lincRNA and mRNA genes categorized by the number of detected intramolecular ligations (x axis).

FIGS. 29A to 29B. RNA Hi-C reads on SNORA14. (FIG. 29A) The intramolecular ligation products mapped to SNORA14. Shown in the black regions are the ligation junctions. The shaded numbers are positions of dominantly represented ligation junctions at the 5′ and the 3′ of the linker. Spatial proximities of 1-6, 1-4, and 5-5 positions are consistent with the sequence predicted secondary structure (FIG. 29B). The arrows point to 3-5 positions which are not close to each other on the sequence predicted secondary structure.

FIGS. 30A to 30C. A putative novel gene that produces structurally stable transcripts. (FIG. 30A) The genomic location and interspecies conservation of the RNA Hi-C predicted novel gene. (FIG. 30B) The intramolecular ligation products mapped to this novel gene. The black regions: ligation junctions. The shaded numbers: positions of dominantly represented ligation junctions. (FIG. 30C) Sequence predicted secondary structures of a long (bottom) and a short (top) transcript produced from this putative gene. The frequency of RNase I digestion on each base (heatmap) correlated with the predicted single-stranded regions (bottom). The ligated positions (arrows) are close on the sequenced predicted secondary structures.

FIG. 31. The inferred structure of a fraction of an mRNA. An RNA Hi-C read pair was superimposed on the secondary structure that was predicted from the sequence of the 27th exon of the Gcn111 gene. The labeled curves correspond to the RNA1 and RNA2 parts of the sequenced chimeric RNA respectively. The shaded curve: linker. Black regions on the shaded curves: ligation junctions. The pointers represent RNase I cutting positions. The cutting-and-ligation process swapped the 5′-3′ order of two RNA fragments: The 5′ fragment (bases 3122-3163, red) and the 3′ fragment (bases 3164-3194, blue) of the mRNA were swapped on the sequenced chimeric cDNA (insert). This will have to be shaded properly by drafting.

FIG. 32. The workflow for recovering chimeric cDNAs in the sequencing library. Local alignments were used to identify any overlap between the forward and the reverse reads in a read pair. Local alignments were used four times (ALIGN1-ALIGN4) to distinguish four types possible configurations of any read pair. Three types (Types 1-3) were included in the output. Type 1 cDNAs were shorter than 100 bp. Type 2 cDNAs were between 100 bp and 200 bp. Type 3 cDNAs were longer than 200 bp. As a quality control, the cDNAs shorter than 100 bp but devoid of the known sequence of P5 or P7 sequencing primers were discarded (Type 4). Each alignment is expressed as local-align (seq1,seq2) {M,m,o,e}′, where ‘seq1’ and ‘seq2’ are two input sequences, ‘M’, ‘m’, ‘o’, ‘e’ are parameters for match, mismatch, open-gap and extend-gap penalties. The output of each alignment (X) included the alignment score (ScoreX), the beginning and end positions of the alignment in the first (BeginPos1_X, EndPos1_X) and the second sequence (BeginPos2_X, EndPos2_X).

FIGS. 33A to 33C. Simulation analysis. (FIG. 33A) A scatter plot of the predicted (y axis) and the true lengths of the cDNAs. The cDNAs with predicted lengths greater than 200 bp were not included, because their exact lengths could not be predicted. (FIG. 33B) The overlap between the predicted and the simulated RNA pairs. (FIG. 33C) The sensitivity and specificity of the predicted RNA pairs for each type of participating RNAs.

FIGS. 34A to 34B. Degree distributions of the entire observed RNA-RNA interaction networks of mouse ES cells (FIG. 34A) and brain (FIG. 34B). The number of nodes (RNA) is inversely proportional to their degrees (number of interactions) in the log scale, characteristic of scale-free networks.

### DEFINITIONS

In the description that follows, a number of terms are used extensively. The following definitions are provided to facilitate understanding of the present alternatives.

As used herein, “a” or “an” may mean one or more than one.

As used herein, the term “about” indicates that a value includes the inherent variation of error for the method being employed to determine a value, or the variation that exists among experiments.

“Ribonucleic acid”, “RNA,” as described herein refers to a nucleic acid that is a polymeric molecule that is implicated in its roles in coding, decoding, regulation, and expression of genes. In some embodiments described herein, the RNA can play an active role within cells by catalyzing biological reactions, controlling gene expression, or sensing and communicating responses to cellular signals. There are several types of RNA. Without being limiting, RNA can include, for example, messenger RNA (mRNA), lincRNA, transposon RNA, pseudoRNA, regulatory RNA, small nuclear RNA (snRNA), small nucleolar RNAs (snoRNA), double stranded RNA, long non coding RNA (long ncRNA or lncRNA), microRNA (miRNAs), short interfering RNAs (siRNAs), Piwi-interacting RNAs (piRNAs), and other types of short RNAs. In some embodiments, a method for generating chimeric RNAs comprising RNAs which interact with one another in a cell is provided. The method can include cross-linking RNA to protein and ligating RNAs cross-linked to the same protein molecule together to form a chimeric RNA. In some embodiments, the RNA is messenger RNA (mRNA), regulatory RNA, small nuclear RNA (snRNA), small nucleolar RNAs (snoRNA), double stranded RNA, long non coding RNA (long ncRNA or lncRNA), microRNA (miRNAs), short interfering RNAs (siRNAs), Piwi-interacting RNAs (piRNAs), or other types of short RNAs known to those skilled in the art.

“Chimeric RNA” as described herein, refers to an RNA complex in which the RNA complex comprises ligated RNAs that are ligated to a same protein molecule and the RNAs are ligated to one another to form this chimeric RNA. In some embodiments, a method for generating chimeric RNAs comprising RNAs which interact with one another in a cell is provided. The method can include cross-linking RNA to protein and ligating RNAs cross-linked to the same protein molecule together to form a chimeric RNA. In some embodiments, the RNA is messenger RNA (mRNA), regulatory RNA, small nuclear RNA (snRNA), double stranded RNA, long non coding RNA (long ncRNA or lncRNA), microRNA (miRNAs), short interfering RNAs (siRNAs), Piwi-interacting RNAs (piRNAs), small nucleolar RNAs (snoRNAs) or other types of short RNAs known to those skilled in the art. In some embodiments, an isolated complex is provided, wherein the isolated complex comprises a chimeric RNA cross-linked to a protein, wherein said chimeric RNA comprises RNAs which interact with one another in a cell.

“Cross-linking,” or “Cross-linked” as described herein, refers to a bond that can link one polymer to another polymer. The cross-linking can occur through covalent or ionic bonds. In some embodiments, RNA is cross-linked to protein by UV induced cross-linking. Irradiation of protein-nucleic acid complexes (a complex comprising protein and nucleic acid, intermediate proteins and nucleic acid or a protein complex and nucleic acid) with ultraviolet light can cause covalent bonds to form between the nucleic acid and proteins that are in close contact with the nucleic acid. In some embodiments herein, RNA is cross-linked to protein by UV radiation.

Cross-linking can also be performed by using a linker as well as other cross-linking methods known to those skilled in the art. In some embodiments, cross-linking can occur by using a probe to link proteins together as well as other cross-linking methods known to those skilled in the art. Cross-linking can be used in synthetic polymer chemistry as well as in the biological sciences. Cross-links can be formed by chemical reactions that are initiated by a variety of conditions. Without being limiting, cross-linking can be initiated, for example by heating, change in pressure, change in pH, UV light, electron beam exposure, gamma radiation and/or other types of radiation known to one skilled in the art. Additionally, cross-linking can also be induced by cross-linking reagents resulting in a chemical reaction that leads to cross-links between two polymers. In some embodiments described herein, the cross-linking is initiated by heat, change in pressure, change in pH, UV light, electron beam exposure, gamma radiation and/or other types of radiation known to those skilled in the art.

Cross-linking reagents can include but is not limited to Amine-to-Amine Cross-linkers, Sulfhydryl-to-Sulfhydryl Cross-linkers, Amine-to-Sulfhydryl Cross-linkers, Sulfhydryl-to-Carbohydrate Cross-linkers, Photoreactive Cross-linkers, Chemoselective Ligation Cross-linking Reagents, In vivo cross-linking reagents and Carboxyl-to-Amine Cross-linkers. In some embodiments, the cross-linking reagent comprises formaldehyde, DSG (disuccinimidyl glutarate), DSS (disuccinimidyl suberate), BS3 (bis(sulfosuccinimidyl)suberate), TSAT (tris-(succinimidyl)aminotriacetate), BS(PEG)5 (PEGylated bis(sulfosuccinimidyl)suberate), BS(PEG)9 (PEGylated bis(sulfosuccinimidyl)suberate), DSP (dithiobis(succinimidyl propionate)), DTSSP (3,3′-dithiobis(sulfosuccinimidyl propionate)), DST (disuccinimidyl tartrate), BSOCOES (bis(2-(succinimidooxycarbonyloxy)ethyl)sulfone), EGS (ethylene glycol bis(succinimidyl succinate)), Sulfo-EGS (ethylene glycol bis(sulfosuccinimidyl succinate)), DMA (dimethyl adipimidate), DMP (dimethyl pimelimidate), DMS (dimethyl suberimidate), DTBP (Wang and Richard's Reagent), DFDNB (1,5-difluoro-2,4-dinitrobenzene), BMOE (bismaleimidoethane), BMB (1,4-bismaleimidobutane), BMH (bismaleimidohexane), TMEA (tris(2-maleimidoethyl)amine), BM(PEG)2 (1,8-bismaieimido-diethyleneglycol), BM(PEG)3 (1,11-bismaleimido-triethyleneglycol), DTME (dithiobismaleimidoethane), SIA (succinimidyl iodoacetate), SBAP (succinimidyl 3-(bromoacetamido)propionate), STAB (succinimidyl (4-iodoacetyl)aminobenzoate), Sulfo-SIAB (sulfosuccinimidyl (4-iodoacetyl)aminobenzoate), AMAS (N-α-maleimidoacet-oxysuccinimide ester), BMPS (N-β-maleimidopropyl-oxysuccinimide ester), GMBS (N-γ-maleimidobutyryl-oxysuccinimide ester), Sulfo-GMBS (N-γ-maleimidobutyryl-oxysulfosuccinimide ester), MBS (m-maleimidobenzoyl-N-hydroxysuccinimide ester), Sulfo-MBS (m-maleimidobenzoyl-N-hydroxysulfosuccinimide ester), SMCC (succinimidyl 4-(N-maleimidomethyl)cyclohexane-1-carboxylate), Sulfo-SMCC (sulfosuccinimidyl 4-(N-maleimidomethyl)cyclohexane-1-carboxylate), EMCS (N-ε-maleimidocaproyl-oxysuccinimide ester), Sulfo-EMCS (N-ε-maleimidocaproyl-oxysulfosuccinimide, ester), SMPB (succinimidyl 4-(p-maleimidophenyl)butyrate), Sulfo-SMPB (sulfosuccinimidyl 4-(N-maleimidophenyl)butyrate), SMPH (Succinimidyl 6-((beta-maleimidopropionamido)hexanoate)), LC-SMCC (succinimidyl 4-(N-maleimidomethyl)cyclohexane-1-carboxy-(6-amidocaproate)), Sulfo-KMUS (N-κ-maleimidoundecanoyl-oxysulfosuccimide ester), SPDP (succinimidyl 3-(2-pyridyldithio)propionate), LC-S PDP (succinimidyl 6-(3(2-pyridyldithio)propionamido)hexanoate), Sulfo-LC-SPDP (sulfosuccinimidyl 6-(3′-(2-pyridyldithio)propionamido)hexanoate), SMPT (4-succinimidyloxycarbonyl-alpha-methyl-α(2-pyridyldithio)toluene), PEG4-SPDP (PEGylated, long-chain SPDP cross-linker), PEG12-SPDP (PEGylated. long-chain SPDP cross-linker), SM(PEG)2 (PEGylated SMCC cross-linker), SM(PEG)4 (PEGylated SMCC cross-linker), SM(PEG)6 (PEGylated, long-chain SMCC cross-linker), SM(PEG)8 (PEGylated, long-chain SMCC cross-linker), SM(PEG)12 (PEGylated, long-chain SMCC cross-linker), SM(PEG)24 (PEGylated, long-chain SWAT; cross-linker), Succinimidyl 3-(2-Pyridyldithio)Propionate PDP), SMCC, Succinimidyl trans-4-(maleimidylmethyl)cyclohexane-1-Carboxylate, B MPH (N-β-maleimidopropionic acid hydrazide), EMCH (N-ε-maleimidocaproic acid hydrazide), MPB (4-(4-N-maleimidophenyl)butyric acid hydrazide), KMUH (N-κ-maleimidoundecanoic acid hydrazide), PDPH (3-(2-pyridyldithio)propionyl hydrazide), ANB-NOS (N-5-azido-2-nitrobenzoyloxysuccinimide), Sulfo-SANPAH (sulfosuccinimidyl 6-(4′-azido-2′-nitrophenylamino)hexanoate), SDA (NHS-Diazirine) (succinimidyl 4,4′-azipentanoate), Sulfo-SDA (Sulfo-NHS-Diazirine) (sulfosuccinimidyl 4,4′-azipentanoate), LC-SDA (NHS-LC-Diazirine) (succinimidyl 6-(4,4′-azipentanamido)hexanoate), Sulfo-LC-SDA (Sulfo-NHS-LC-Diazirine) (sulfosuccinimidyl 6-(4,4′-azipentanamido)hexanoate), SDAD (NHS-SS-Diazirine) (succinimidyl 2-((4,4′-azipentanamido)ethyl)-1,3′-dithiopropionate), Sulfa-SDAD (Sulfo-NHS-SS-Diazirine) (sulfosuccinimidyl 2-((4,4′-azipentanamido)ethyl)-1,3′-dithiopropionate), ATFB, SE, 4-Azido-2,3,5,6-Tetrafluorobenzoic Acid, Succinimidyl Ester, SDA (NHS-Diazirine) (succinimidyl 4,4′-azipentanoate), SPB (succinimidyl-[4-(psoralen-8-yloxy)]-butyrate), L-Photo-Leucine, L-Photo-Methionine, ManNAz (N-azidoacetylmannosamine tetraacylated), GalNAz (N-azidoacetylgalactosamine, tetraacylated), DCC (dicyclohexylcarbodiimide), DyLight™ 550-Phosphine, DyLight™ 650-Phosphine, EZ-Link™ Phosphine-PEG3-Biotin, EZ-Link™ Phosphine-PEG4-Desthiobiotin, EDC (1-ethyl-3-(3-dimethylaminopropyl)carbodiimide hydrochloride), NHS (N-hydroxysuccinimide), Sulfo-NHS (N-hydroxysulfosuccinimide), Sulfo-NHS (N-hydroxysulfosuccinimide), Sulfo-NHS (N-hydroxysulfosuccinimide) or Sulfo-NHS (N-hydroxysulfosuccinimide).

“Immobilization” as described herein, refers to the capturing of a molecule, wherein the capturing is performed by a first molecule that is specific for a specific molecule or a label. In some embodiments, the immobilization is performed by attachment of a capture molecule onto a solid support. The solid support can be a bead or a column. In some embodiments, the solid support comprises a streptavidin molecule for capturing a molecule such as streptavidin or a portion thereof. In some embodiments. the protein is biotinylated at a cysteine residue.

“Fragmenting” as described herein, can refer to digesting or breaking apart of a nucleic acid. In some embodiments of the methods described herein, an RNA is fragmented by an enzyme. RNA degradation can be performed by many types of nucleases. For example, ribonuclease (RN Fuse), is a type of nuclease that can catalyze the degradation of RNA into smaller components. RNAses can be divided into endoribonucleases and exoribonucleases. In some embodiments, a method for generating chimeric RNAs comprising RNAs which interact with one another in a cell is provided, wherein the method comprises cross-linking RNA to protein and ligating RNAs cross-linked to the same protein molecule together to form a chimeric RNA. In some embodiments, cross-linking of RNA to protein is performed on an intact cell or in a cell lysate. In some embodiments, cross-linking comprises UV cross-linking. In some embodiments, the method further comprises associating said protein with an agent which facilitates immobilization of said protein on a surface. In some embodiments, said agent which facilitates immobilization, comprises biotin. In some embodiments, the protein is biotinylated at a cysteine residue. In some embodiments, the method further comprises fragmenting said RNAs cross-linked to the same protein molecule. In some embodiments, said fragmenting comprises contacting said RNAs cross-linked to the same protein molecule with an RNAse under conditions which facilitate partial digestion of said RNAs.

“Biotin” as described herein, refers to a water soluble B vitamin that is also known as vitamin H or coenzyme R. In several embodiments described herein, biotin can be used to label RNA for capture by a streptavidin molecule on a solid support, such as a bead. In some embodiments, a method for generating chimeric RNAs comprising RNAs which interact with one another in a cell is provided, wherein the method comprises cross-linking RNA to protein and ligating RNAs cross-linked to the same protein molecule together to form a chimeric RNA. In some embodiments, cross-linking of RNA to protein is performed on an intact cell or in a cell lysate. In some embodiments, cross-linking comprises UV cross-linking. In some embodiments, the method further comprises associating said protein with an agent which facilitates immobilization of said protein on a surface. In some embodiments, said agent which facilitates immobilization, comprises biotin. In some embodiments, the protein is biotinylated at a cysteine residue. In some embodiments, the method further comprises fragmenting said RNAs cross-linked to the same protein molecule. In some embodiments, said fragmenting comprises contacting said RNAs cross-linked to the same protein molecule with an RNAse under conditions which facilitate partial digestion of said RNAs. In some embodiments, the method further comprises linking said RNAs cross-linked to the same protein molecule to an agent which facilitates recovery of said RNAs. In some embodiments, said linking comprises ligating the ends of said RNAs to said agent. In some embodiments, said agent which facilitates recovery of said RNAs comprises a nucleic acid. In some embodiments, said nucleic acid comprises a nucleic acid having biotin thereon. In some embodiments, said linking of said nucleic acid having biotin thereon to said ends of said RNAs comprises ligating said nucleic acid having biotin thereon to the 5′ ends of said RNAs prior to ligating said RNAs cross-linked to the same protein molecule together to form a chimeric RNA. In some embodiments, the method further comprises removing said biotin from the 5′ region of said chimeric RNA. In some embodiments, the method further comprises recovering said chimeric RNAs. In some embodiments, the method further comprises fragmenting said chimeric RNAs.

“Protein” as described herein refers to a macromolecule comprising one or more polypeptide chains. A protein can therefore comprise of peptides, which are chains of amino acid monomers linked by peptide (amide) bonds, formed by any one or more of the amino acids. A protein or peptide can contain at least two amino acids, and no limitation is placed on the maximum number of amino acids that can comprise the protein or peptide sequence. Without being limiting, the amino acids are, for example, arginine, histidine, lysine, aspartic acid, glutamic acid, serine, threonine, asparagine, glutamine, cysteine, cystine, glycine, proline, alanine, valine, hydroxyproline, isoleucine, leucine, pyrolysine, methionine, phenylalanine, tyrosine, tryptophan, ornithine, S-adenosylmethionine, and selenocysteine. A protein can also comprise non-peptide components, such as carbohydrate groups. Carbohydrates and other non-peptide substituents can be added to a protein by the cell in which the protein is produced, and will vary with the type of cell. Without being limiting, proteins can function within organisms by catalyzing metabolic reactions, DNA replication, responding to stimuli, and transporting molecules from one location to another. For example, the proteins can be an enzyme, a transmembrane protein, and antibody, a small biomolecule for transport, a receptor or a hormone. In some embodiments, a method for generating chimeric RNAs comprising RNAs which interact with one another in a cell is provided, wherein the method comprises cross-linking RNA to protein and ligating RNAs cross-linked to the same protein molecule together to form a chimeric RNA. In some embodiments, the protein is an enzyme. In some embodiments, the protein is involved in transport, or in catalysis of metabolic reactions.

“Interactome” as described herein, refers to a whole set of molecular interactions in a particular cell. The term specifically refers to physical interactions among molecules (such as those among proteins, also known as protein-protein interactions) but can also describe sets of indirect interactions among genes (genetic interactions) such as RNA-RNA interactions or interactions between one or more RNA and a protein molecule. In some examples, the interactomes can be displayed as graphs. In some embodiments, the present methods and compositions map substantially all protein-assisted RNA-RNA interactions in one assay. In some embodiments described herein the methods have been applied to produce the first global map of an RNA interactome. In some embodiments, an interactome is produced from a specific cell. In some embodiments, the cell is from a human. In some embodiments, the cell is a cancer cell, a tumor cell, a lymphocyte or an immune cell. In some embodiments, the interactome can be used to determine or predict a disease pathway.

A “protein complex” as defined herein, refers to a group or two or more associated proteins or polypeptide chains and can also be referred to as a “multiprotein complex”. In some embodiments, a complex comprising a nucleic acid(s) bound to a protein complex is provided. In some embodiments, the nucleic acid(s) is RNA.

“Protein intermediates” as defined herein refers to proteins that can bind to one another off and on during a process or a specific pathway, and can also be referred to as “protein binding intermediates.” Without being limiting, examples in which protein intermediates can be seen binding can include processes such as transcription, translation and metabolic pathways. Without being limiting, examples of protein binding intermediates can include polymerases, nucleic acid binding proteins, RNA recognition motic proteins, heterogeneous ribonucleoprotein particles, and other protein binding intermediates known to those skilled in the art. In some embodiments, a complex comprising a nucleic acid(s) bound to protein intermediate(s) is provided. In some embodiments, the nucleic acid(s) is RNA. In some embodiments, the protein intermediates interact with other protein intermediates, thus forming a protein complex, wherein the protein complex comprises protein intermediates.

### DETAILED DESCRIPTION

Disclosed herein are methods and compositions for identifying direct RNA-RNA interactions in a cell. In some embodiments, the methods and compositions can be used to identify at least about 100, at least about 500, at least about 1000 or more than about 1000 RNA-RNA interactions in the cell. In some embodiments, the methods and compositions can be used to identify about 100, about 200, about 300, about 300, about 500, about 600, about 700, about 800, about 900, about 1000, about 2000, about 3000, about 4000, about 5000, about 6000, about 7000, about 8000, about 9000 or about 10,000 RNA-RNA interactions or any other number of RNA-RNA interactions between any two of these aforementioned values. In other embodiments, the methods and compositions can be used to identify substantially all of the direct RNA-RNA interactions in the cell. For example, the methods and compositions can be used to identify at least about 70%, at least about 80%, at least about 90% or more than about 90% of the direct RNA-RNA interactions in the cell. In some embodiments, the methods and compositions can be used to identify at least about 70%, at least about 80%, at least about 90% or about 100% of the direct RNA-RNA interactions in the cell, or any other percent between any two of the aforementioned values described. This method does not rely on knowledge of any specific RNA sequence and one of the benefits is identifying unknown RNA-RNA interactions.

Only about 5% of the genome codes for RNA that is translated into a protein. About 50% of the genome is transcribed into RNA, including non-coding RNA (ncRNA) such as microRNA and long ncRNA (longer than 200 nt). ncRNA often interacts with other RNA, via protein-associated interactions. Accordingly, direct RNA-RNA interactions can be identified using a protein-based capture method. In some embodiments, the direct RNA-RNA interactions can be identified using a protein-based capture method.

Although RNA-RNA interactions are essential for RNA's regulatory functions, there is yet no technology to globally survey them. The available technologies including HITS-CLIP (Nature 460, 479-486) and CLASH (Cell 153, 654-665) can only map the RNAs attached to a selected protein. Such one-protein-at-a-time approaches cannot map the entire RNA interactome.

In some embodiments, the present methods and compositions map substantially all protein-assisted RNA-RNA interactions in one assay. In some embodiments described herein the methods have been applied to produce the first global map of an RNA interactome. In some embodiments, the present methods and compositions circumvents the requirement for a protein-specific antibody or the need to express a tagged protein. This allows for an unbiased mapping of the RNA interactome. To our knowledge, other methods can only work with one RNA-binding protein at a time. The embodiments described herein, lead to a surprising outcome in which RNA-RNA interactions can be determined for multiple RNA binding proteins.

In some embodiments, the present methods and compositions analyze the endogenous cellular condition without introducing any exogenous nucleotides or protein-coding genes (CLASH) prior to cross-linking. Rather than requiring a transformed cell line (CLASH), some embodiments are generally applicable to analyze any cell type or tissue.

In some embodiments, the present methods and compositions overcome an important drawback of HITS-CLIP. HITS-CLIP inferred RNA-RNA interactions did not necessarily occur in the cells analyzed. This is because any two RNAs that co-appeared in HITS-CLIP could have resulted from the independent attachment of either RNA to different copies of the targeted protein. However, in some embodiments, the present methods and compositions reliably represent the physical interactions of RNAs.

The RNA interactome in mouse embryonic stem (ES) cells have been mapped and herein the new findings show:
1. Long RNAs often interact with each other. There are thousands of mRNA-mRNA interactions and hundreds of lincRNA-mRNA, transposonRNA-mRNA, pseudogeneRNA-mRNA interactions in mouse ES cells.
2. Interactions between long RNAs frequently use a small fraction of the transcripts. In analogy to protein interaction domains, the notion of RNA interaction sites is proposed herein. RNA interaction sites utilize base pairing to facilitate interactions of long RNAs, suggesting a new type of trans regulatory sequences. These trans regulatory sequences are more evolutionarily conserved than other parts of transcripts.
3. The RNA interactome is a scale-free network, with several highly connected lincRNA and mRNA hubs. In an exemplary embodiment, an interaction between two hubs, Malat1 lincRNA and Slc2a3 mRNA has been experimentally verified, using two-color single molecule RNA-FISH.
4. Essentially every expressed snoRNA is enzymatically processed into a miRNA-like small RNA and interacted with mRNAs in RISC complex.

Although some embodiments of the present methods and compositions can be used for mapping inter-molecule interactions, they can also reveal unique information concerning RNA structure. The intra-molecule reads of RNA Hi-C provided spatial proximity information for various segments of an RNA. As such, this is the first time that such information has become available in a high-throughput manner. Additionally, the single stranded regions of every RNA were obtained during the same assay as a byproduct. In an exemplary embodiment, an RNA was bent by a protein, and such quaternary structure was captured by intra-molecule reads of RNA Hi-C.

In some embodiments, the method comprises: (1) cross-linking RNA1 and RNA2 to a protein (or to a protein intermediate or a protein complex) to form a complex, (2) labelling protein (e.g. Biotin), (3) fragmenting RNA, (4) capturing labelled protein (e.g. biotin-streptavidin-bead), (5) ligating a biotin-tagged RNA linker to the 5′ end of RNA1 and RNA2, (6) performing proximity ligation to ligate RNA1-linker-RNA2 forming a chimera, (7) protease treating the complex to release RNA1-linker-RNA2 chimera (DNAse treat), (8) hybridizing with DNA probe complementary to biotin-tagged RNA linker and treating with T7 exonuclease to remove non-ligated biotin-tagged RNA linker, (9) fragmenting nucleic acids to about 150 nt to assist with ultimate sequencing, (10) capturing RNA1-linker-RNA2 chimera using streptavidin bead, (11) converting RNA1-linker-RNA2 to cDNA and sequencing at least a portion of the cDNA. In some embodiments, bioinformatics is used to identify RNA1 and RNA2.

The present methods and compositions find application in a variety of contexts, including use by RNA therapeutic companies searching for new therapeutic targets, use by researchers to investigate RNA-RNA interactions and development by device and reagent companies for research and discovery devices.

Non-coding RNAs (ncRNAs) are involved in a wide range of cellular processes, including the regulation of gene expression. MicroRNAs (miRNAs) and long ncRNAs (lncRNAs) are two classes of ncRNAs with known regulatory functions. The ability of these ncRNAs to modulate gene expression at post-transcriptional or epigenetic level provide new opportunities for ncRNA based therapeutics. Identification of direct interactions among ncRNAs and messenger RNAs (mRNAs) is an inevitable step to understand the regulatory roles of ncRNAs. MiRNA and lincRNA targetings are only small portions of interactions that can be detected by technology described in the embodiments herein, it is also designed to discover the potential regulatory functions of other ncRNAs. However, the market of diagnosis and therapeutics driven only by these two classes of ncRNAs is already going to be significant.

MiRNAs are a group of non-coding ribonucleic acids that serve as key regulators of gene expression. Recent studies have further revealed the importance of miRNAs in diseases, especially in cancer, cardiovascular, and neurological diseases. Large-scale cloning efforts have revealed the abundance and variety of miRNAs. The human genome has been estimated to encode up to 1000 miRNAs and these are predicted to regulate a third of all genes. In neurological processes, miRNAs are key mediators of both central nervous system (CNS) development and plasticity. Increasing evidence indicates that miRNAs are involved in neurological disorders as diverse as traumatic spinal cord injury, traumatic brain injury, Alzheimer's disease, Parkinson's disease and Huntington's disease. A potent feature of miRNA-based regulation is the ability of single miRNAs to regulate multiple functionally related mRNAs, as exemplified by the liver-specific miR-122, which regulates multiple metabolic genes. On average, a given miRNA can regulate several hundred transcripts whose effector molecules function at various sites within cellular pathways and networks. Because of this, miRNAs are able to switch instantly between cellular programs and are therefore often viewed as master regulators of the human genome.

It was only 10 years ago that the first human miRNA was discovered, and yet a miRNA-based therapeutic has already entered Phase 2 clinical trials (miR-122 antagonist, SPC3649, developed by Santaris, is administered to HCV patients to block replication of the virus). This rapid progress from discovery to development reflects the importance of miRNAs as critical regulators in human disease, and holds the promise of yielding a new class of therapeutics that could represent an attractive addition to the current drug pipeline.

The principles that apply to developing miRNA-based therapies remain the same as for other targeted therapies that take the path from drug target to drug. For instance, target identification and validation are key to selecting miRNAs that are causally involved in the disease process. Furthermore, diligent drug development is necessary to assure satisfactory efficacy, specificity and lack of toxicity. However, since miRNAs constitute a class of drug targets unrelated to any others, new ancillary technologies and methods are also required. A critical missing piece in harnessing the therapeutic potentials of miRNAs is an assay to identify the target mRNAs of miRNAs. In some embodiments, the present methods and compositions can be used to develop therapeutic strategies and compositions.

The market of cancer therapy is close to 100 billion currently and is predicted to expand exponentially in the next five years. microRNA based therapeutics have become the leading edge of this field, and according to some analysts predicted to occupy a market space worth $7.5 billion, based on a $150 million market per therapeutic miRNA and assuming 50 miRNAs with therapeutic potential are approved for use.

In some embodiments, the present compositions and methods provide a missing piece that cannot be circumvented in any miRNA-driven therapeutic applications. Other applications of the present methods and compositions include therapeutic applications in neurological disorders and research labs.

lincRNAs are non-protein coding transcripts longer than 200 nts which can mediate interactions between epigenetic remodeling complexes and chromatin. A deeper understanding of lncRNA function in human cancer will not only expand the number of potential target cancer genes, but can also facilitate development of novel anti-cancer therapies, such as gene regulation mediated by antisense RNAs or targeting lncRNA-protein interactions. With a deeper understanding of the roles of lncRNA in normal and diseases states, it is believed that lncRNAs can also be used as diagnostic or predictive biomarkers. For example, the lncRNA HOTAIR is increased in expression in primary breast tumors and metastases, and its expression level in primary tumors is a powerful predictor of eventual metastasis and death. Moving closer to the clinics, a lncRNA called prostate cancer antigen 3 (PCA3), which is highly overexpressed in prostate cancer, happens to be found in urine, making for easy testing. A commercial kit, called the Progensa PCA3 test, which is the first urine-based molecular test to help determine a need for repeat prostate biopsies, has been approved for clinical application by the FDA recently. The disease-regulating importance of lncRNAs is not limited to cancer. They also play important roles in heritable conditions, notes Gibb, in which lncRNA deregulation has been associated with brachydactyly and HELLP syndrome. Another lncRNA was shown to stabilize the mRNA for a crucial enzyme in the Alzheimer's disease pathway. Increasing evidence suggests lncRNAs are closely associated with major human diseases, and can have better performance in disease diagnosis and prognosis compared with protein-coding RNAs. Furthermore, the majority of currently available drugs and tool compounds exhibit an inhibitory mechanism of action and there is a relative lack of pharmaceutical agents that are capable of increasing the activity of effectors or pathways for therapeutic benefit. Indeed, the upregulation of many genes, including tumor suppressors, growth factors, transcription factors and genes that are deficient in various genetic diseases, would be desired in specific situations. Many reports suggest that lncRNAs can often be suppressed by RNAi triggers. Targeting lncRNAs by RNAi that silence other genes can activate gene expression. In some embodiments, the methods and compositions can be used to detect the presence or absence of upregulated genes in cells of interest. In some embodiments the cells comprise tumor cells, cancer cells or immune cells. In some embodiments, the methods can be used to identify or predict disease or disease outcome by evaluation of a transcriptome comprising the information of genes upregulated.

Thus, in some embodiments, the present methods and compositions can be utilized by companies in the miRNA therapeutics market who use miRNA mimics to normalize gene regulatory network on cancerous cells, or treat cardiovascular and muscle disease. In an exemplary embodiment, the present methods and compositions can be utilized to validate candidate products and also to search for new targets.

In some embodiments, the present methods and compositions can be used for manufacturing RNA Hi-C kits. In other embodiments, the present methods and compositions can be used to provide oligonucleotides for research. For example, the present methods and compositions can be utilized in the context of large lncRNA-targeting RNAi trigger libraries. In some embodiments, the present methods and compositions are used to identify potential lncRNA candidates for RNAi targeting.

One embodiment provides a technology to map out RNA-RNA interactions in cells. In one embodiment, the methods and compositions unbiasedly map out substantially all RNA-RNA interactions in one experiment, and provide one-to-one resolution (which RNA interacts with which RNA). Some embodiments include a novel experimental component and a new computational strategy. Starting from the cells of a certain cell type, some embodiments map out a list of directly interacting RNAs of this cell type. The present methods and compositions have been applied to mouse embryonic stem cells and identified 4049 RNA-RNA interactions using one experiment. In one embodiment, the experimental component takes these cells as input, transforms substantially all direct RNA-RNA interactions into chimeric RNA molecules, and sequences these chimeric RNAs using pair-end sequencing. Some embodiments comprise (1) immobilization of all protein-RNA complexes (a complex comprising protein and nucleic acid, intermediate proteins and nucleic acid or a protein complex and nucleic acid) to magnetic beads; (2) proximity-based ligation of interacting RNAs; (3) selective purification of chimeric RNA molecules; (4) high-throughput sequencing of chimeric transcript. In an embodiment described herein, the method can further comprise using a bioinformatic program to take these sequencing data as input, and produce a list of high-confidence RNA-RNA interactions.

Currently, there are no efficient methods that can directly assay substantially all RNA-RNA interactions in a cell type at once. There are two kinds of methods which exist to partially achieve this goal, both with weakness. First, experimentally characterizing the targets of only one miRNA/lincRNA in vivo is considered as a pioneering technology [Lal et al., 2011; Baigude et al., 2012; Kretz et al., 2013]. Second, other technologies like HITS-CLIP and CLASH that can detect targets of many miRNAs also have restrictions. One major common restriction is that they both concentrated on miRNAs, which only comprise a small portion of RNAs. Thus, these technologies are not able to reveal the majority of RNA-RNA interactions. Furthermore, each technology has its own specific weakness.

High-throughput sequencing of RNA isolated by cross-linking immunoprecipitation (HITS-CLIP) is the most reliable method for genome-wide analyses of miRNA targets currently [Chi et al., 2009]. HITS-CLIP allows the identification of the total collection of miRNAs present in a tissue, as well as all the total collection of mRNAs regulated by miRNAs. However direct pairing of a miRNA to its target mRNAs cannot be directly deduced from HITS-CLIP. In other words, HITS-CLIP does not directly inform which miRNA regulates which mRNAs (no one-to-one information).

A recent method called CLASH (cross-linking, ligation, and sequencing of hybrids) could allow direct observation of miRNA-target pairs. However, the number of interactions is still small as compared to number of sequencing reads: only 2% of sequenced reads are chimeric, 98% are still single reads. This requires much deeper sequencing coverage or preparation of multiple samples to obtain enough coverage of miRNA-mRNA interactions.

In some embodiments, the present methods and compositions include experimental and computational components to make and enrich RNA chimeras so that an unbiased, genome-wide, direct assay for information of all RNA-RNA interactions could be mapped.

In some embodiments, the present methods and compositions provide:
1. Direct assaying of all RNA-RNA interactions at one-to-one resolution using chimeric RNAs.
2. The utilization of specific linkers to enhance efficiency of ligation and accuracy of interaction identification.
3. Selective purification of desirable chimeric RNA-RNA products is achieved by removal of unligated products and biotin pull-down.
4. Enhanced efficiency of library preparation for high throughput sequencing by the use of ssDNA Circligase to attach sequencing adaptor instead of RNA ligase.

In some embodiments, the present methods and compositions are able to:
1. Identify the chimeric RNA sequences from all the sequence reads produced by the experimental step;
2. Transform those chimeras into annotated RNA clusters;
3. Identify strong direct interactions among these RNA clusters using a statistical test.

As previously noted, some technologies characterize the targets of only one miRNA/lincRNA in vivo (for example, Lal et al., 2011; Baigude et al., 2012; RNA interactome analysis).

As previously noted, some technologies can detect targets of many miRNAs, but are restricted to miRNA (for example, HITS-CLIP, PAR-CLIP, which also lack direct one-to-one information and CLASH, which provides only a small portion of chimeric RNAs). As such the present embodiments described herein lead to an advantage relative to the previous methods by not restricting the RNA is to a small subset such as miRNA.

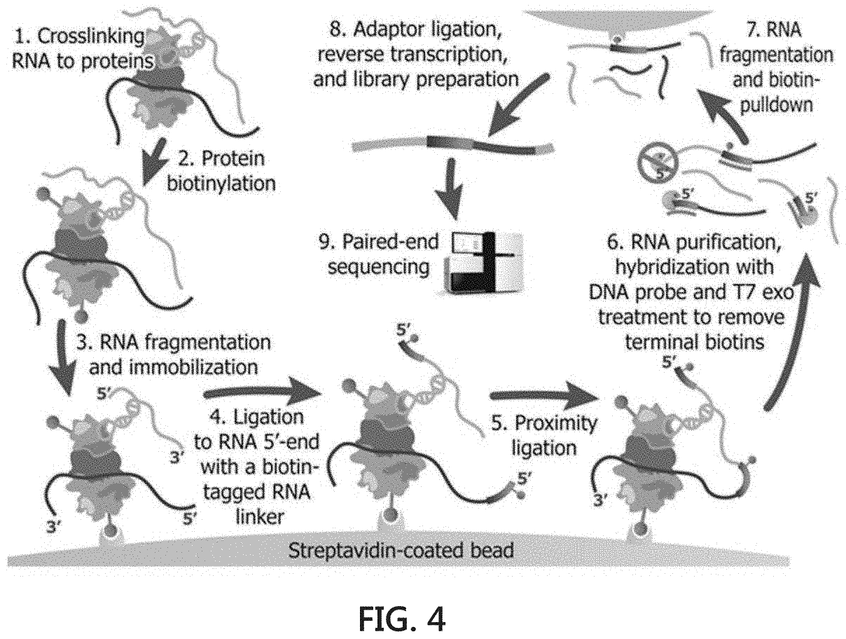

One exemplary embodiment is illustrated in FIG. 4. Briefly, cells are cross-linked in vivo by UV cross-linking. UV cross-linking has the advantage that RNA is covalently bound to the protein of interest but proteins are not cross-linked to each other. The covalent interaction formed between RNA and the protein allows stringent purification of the cross-linked RNA fragments. Cells are lysed and the lysate is subjected to partial RNase digestion by RNase I. Also, the cysteine residues are biotinylated on proteins. The proteins including protein-RNA complexes (a complex comprising a protein and nucleic acid, intermediate proteins and nucleic acid or a protein complex and nucleic acid, wherein the nucleic acid is RNA) are immobilized on streptavidin beads. The 5′ end of the RNA is then ligated with a biotin-tagged RNA linker (24 nt) to facilitate subsequent selective purification of chimeric RNAs. Next, proximity-based ligation is carried out on beads under dilute conditions that favor ligations between cross-linked RNA fragments. Protein-RNA complex (a complex comprising a protein and nucleic acid, intermediate proteins and nucleic acid or a protein complex and nucleic acid, wherein the nucleic acid is RNA) is then eluted from streptavidin beads and RNA is recovered by digesting the bound protein. Eluted RNAs are subjected to rigorous DNase treatment to eliminate DNA contamination. Purified RNAs are then hybridized with a DNA probe that is complementary to the 24 nt RNA linker, and treated with T7 exonuclease to remove the non-ligated biotinylated RNA linkers. As a result, only the successfully ligated chimeric RNAs contain a biotin-tagged linker at the junction. This chimeric RNA library is fragmented again to an average of 150 nucleotides, and the ligation junctions are pulled-down with streptavidin-coated magnetic beads. The end product is a library of ˜150 nt chimeric RNAs. This library is expected to be enriched with chimeras in the form of R1-linker-R2, where R1 and R2 are fragments of interacting RNAs. This library is converted into cDNAs and sequenced with paired-end next-generation sequencing.

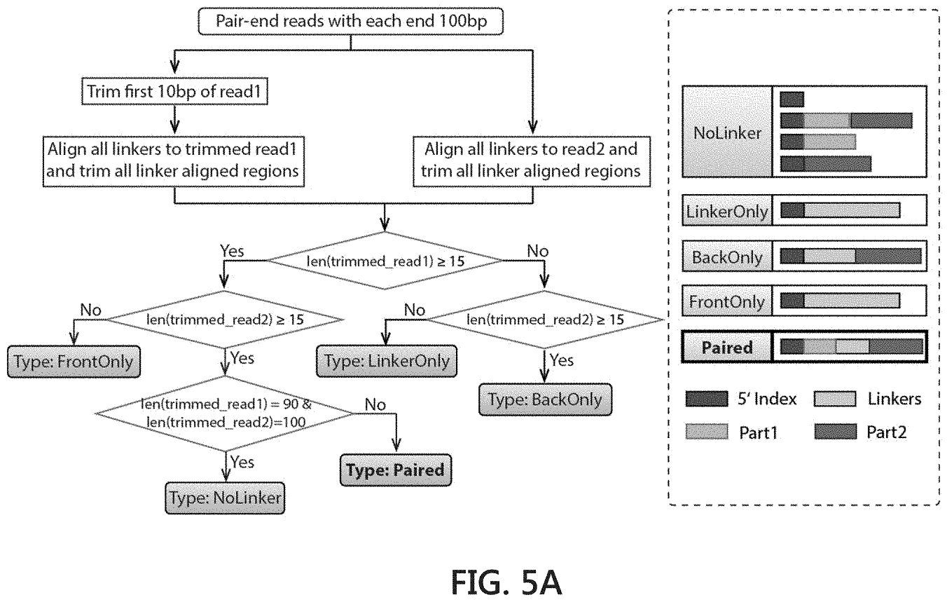

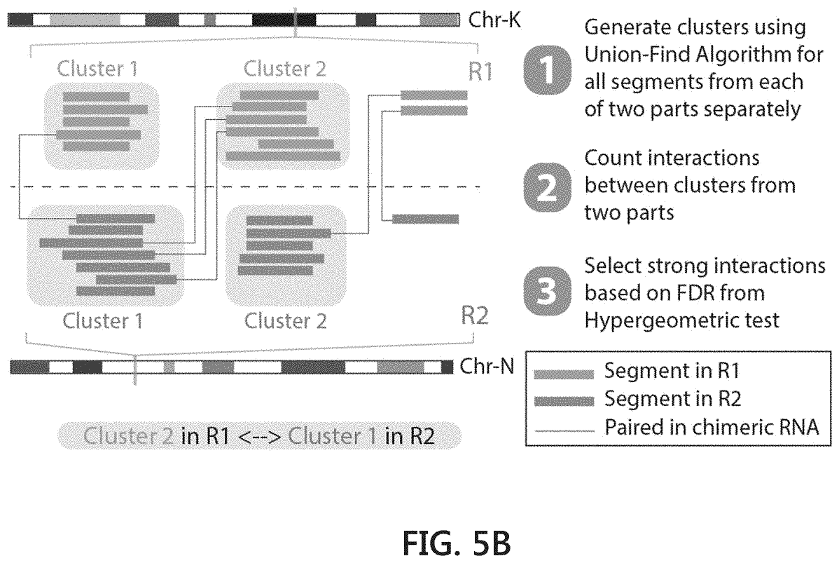

One exemplary embodiment of the bioinformatics analysis of the sequenced cDNAs is illustrated in (FIGS. 5A to 5B). First, PCR duplicates are removed for reads with both ends completely the same as another. Then, the fragments sent for sequencing are recovered and fragment lengths were estimated based on BLAST alignment between two ends of each read pair. From that, the informative chimeric RNAs with the R1-linker-R2 configuration are selected, where R1 and R2 are fragments of the interacting RNAs (FIG. 5A). After chimeric RNAs are collected, R1 and R2 fragments are aligned back to the genome and clusters supported by large numbers of overlapped aligned reads are generated for R1 and R2 pools in parallel (using Union-Find algorithm).

Next, a hypergeometric test are developed to identify strong interactions between clusters within R1 and R2 pools based on the number of ligated chimeras (R1-linker-R2). Different types of strong interactions are determined by genomic annotations of clusters in R1 and R2 pools. (FIG. 5B)

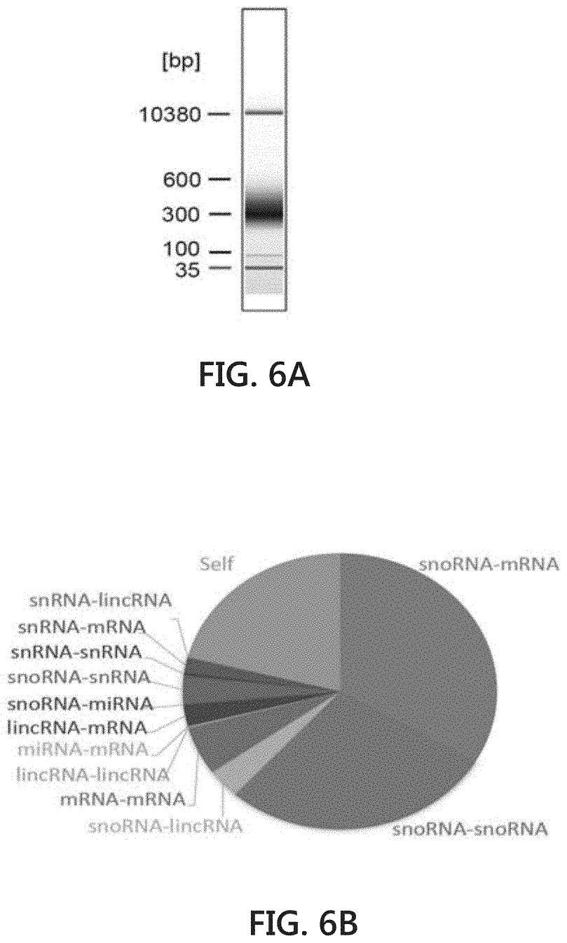

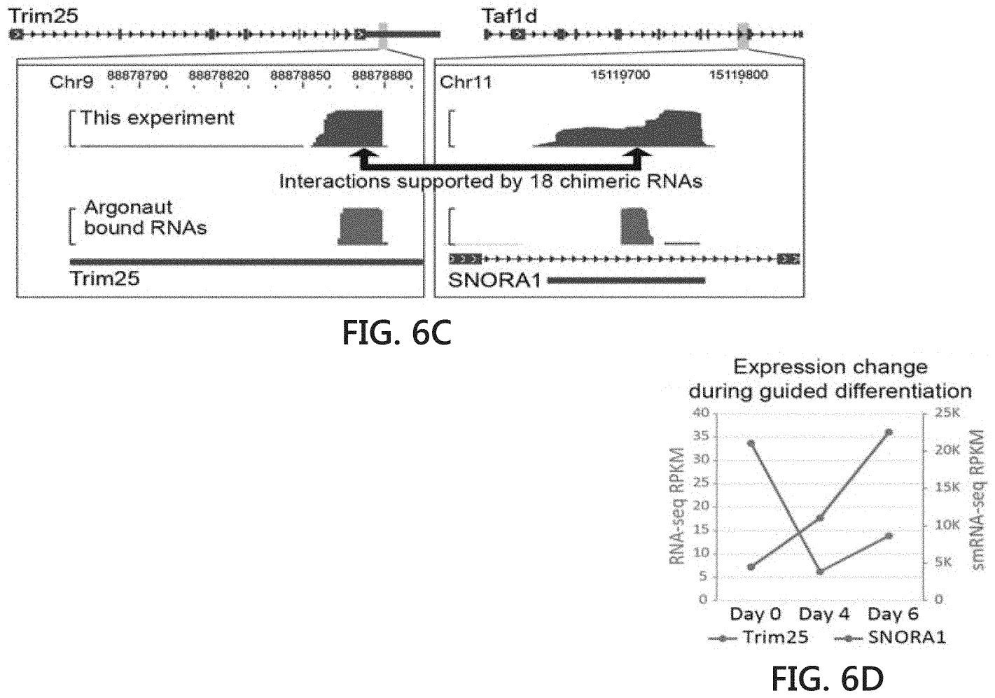

Two independent experiments using mouse embryonic stem (ES) cells have been conducted. These two experiments produced comparable results. The cDNAs ranged from 75 to 200 nts (FIG. 6A, subtract 128 nt primers), which produced ˜24 million non-redundant pair-end reads. The chimeric RNAs of the form R1-linker-R2 were identified (2.4 million). A total of 4049 interactions were identified by hypergeometric tests and categorized different types of interactions (FIG. 6B), in which snoRNA-mRNA interactions were the most abundant. In 242 interactions, snoRNAs targeted the 3′UTRs of mRNAs, supporting a recently proposed hypothesis that snoRNAs can be processed into smaller molecules and function like miRNAs [Brameier et al., 2011; Scott et al., 2011]. For example, 18 non-redundant chimeric RNAs linked the SNORA1 snoRNA with the 3′UTR of Trim25 mRNA (FIG. 6C). Argonaute protein pull-down followed by RNA sequencing (CLIP-seq) data [Lueng et al., 2011] confirmed that both SNORA1 and Trim25 were attached with Argonaute (FIG. 6C). The time-course analysis of ES cell differentiation [Shu et al., 2012] confirmed a reverse correlation (FIG. 6D), consistent with the idea that one RNA represses the other.

This proof of principle experiment with our technology produced a list of 4049 pairs of interacting RNAs. The top 10 interactions, based on p-values and number of supporting read-pairs, are provided in Table 1.

TABLE 1
The top 10 RNA-RNA interactions identified by RNA-Stich-Seq
in embryonic stem cells. Each row provides the information
of a pair of interacting RNAs, named as interacting RNA 1 and interacting RNA 2.
The number of chimeric RNAs, which were formed
due to this interacting pair and were reflected as pair-end sequencing reads,
is provided in the last column. Double ended arrows
indicate direct interactions.

| Interacting RNA 1 | | | | Interacting RNA 2 | | | Evidence |
|---|---|---|---|---|---|---|---|
| Genome_loc-1 | Type-1 | Name-1 | | Genome_loc-2 | Type-2 | Name-2 | #pair-end |
| chr1:95404306- | mRNA | Sept2 | [symbol] | chr17:24857673- | snoRNA | Snora64 | 64 |
| 95404378 | | | | 24857853 | | | |
| chr18:33954812- | snoRNA | AC150277 | [symbol] | chr1:37509195- | mRNA | Mgat4a | 33 |
| 33954959 | | | | 37509268 | | | |
| chr8:108188064- | mRNA | Ctcf | [symbol] | chr11:53229198- | mRNA | Aff4 | 26 |
| 108188144 | | | | 53229257 | | | |
| chr5:30111576- | mRNA | Dnajb6 | [symbol] | chr10:82773768- | mRNA | Slc41a2 | 20 |
| 30111620 | | | | 82773822 | | | |
| chrX:166133701- | snoRNA | SNORA51 | [symbol] | chr3:94811096- | mRNA | Zfp687 | 18 |
| 166133796 | | | | 94811204 | | | |
| chr8:114243866- | mRNA | Bcar1 | [symbol] | chr7:4088679-4088753 | mRNA | Leng8 | 17 |
| 114243973 | | | | | | | |
| chr6:146411883- | mRNA | Itpr2 | [symbol] | chr11:77996556- | snoRNA | Snord42b | 15 |
| 146411981 | | | | 77996634 | | | |
| chr11:62418011- | snoRNA | Snord65 | [symbol] | chr11:76622267- | mRNA | Cpd | 14 |
| 62418098 | | | | 76622375 | | | |
| chr6:115757981- | snoRNA | Snora7a | [symbol] | chr14:56505700- | mRNA | Khnyn | 14 |
| 115758175 | | | | 56505732 | | | |
| chrX:34622893- | snoRNA | Snora69 | [symbol] | chr15:78982084- | mRNA | Polr2f | 14 |
| 34623042 | | | | 78982165 | | | |

Many biological processes are regulated by RNA-RNA interactions (Kretz, M. et al. Control of somatic tissue differentiation by the long non-coding RNA TINCR. Nature 493, 231-235, doi:10.1038/nature11661 (2013)), nonetheless it remains formidable to analyze the entire RNA interactome. In an exemplary embodiment, a method, RNA Hi-C, was developed to map protein-assisted RNA-RNA interactions in vivo. By circumventing the selection for a specific RNA-binding protein (Hafner, M. et al. Transcriptome-wide identification of RNA-binding protein and microRNA target sites by PAR-CLIP. Cell 141, 129-141, doi:10.1016/j.cell.2010.03.009 (2010); Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009); Helwak, A., Kudla, G., Dudnakova, T. & Tollervey, D. Mapping the human miRNA interactome by CLASH reveals frequent noncanonical binding. Cell 153, 654-665, doi:10.1016/j.cell.2013.03.043 (2013); Kudla, G., Granneman, S., Hahn, D., Beggs, J. D. & Tollervey, D. Cross-linking, ligation, and sequencing of hybrids reveals RNA-RNA interactions in yeast. Proceedings of the National Academy of Sciences of the United States of America 108, 10010-10015, doi:10.1073/pnas.1017386108 (2011)), the approach vastly expanded the identifiable portion of the RNA interactome. Use of this technology, allowed mapping of the RNA interactome in mouse embryonic stem cells, which was composed of 46,780 RNA-RNA interactions. The RNA interactome was a scale-free network, with several lincRNAs and mRNAs emerging as hubs. An interaction was validated between two hubs, Malat1 and Slc2a3, using single molecule RNA fluorescence in situ hybridization. Base pairing was observed at the interaction sites of long RNAs, and was particularly strong in transposon RNA-mRNA and lincRNA-mRNA interactions. This revealed a new type of regulatory sequences acting in trans. Consistent with their hypothesized roles, the RNA interaction sites were more evolutionarily conserved than other regions of the transcripts. RNA Hi-C also provided new information on RNA structures, by simultaneously revealing the footprint of single stranded regions and the spatially proximal sites of each RNA. Thus, the unbiased mapping of the protein-assisted RNA interactome with minimum perturbation of cell physiology is advantageous to previous methods and will greatly expand the capacity to investigate RNA functions.

Interactions between RNA molecules exert key regulatory roles and are often mediated by RNA binding proteins (Ray, D. et al. A compendium of RNA-binding motifs for decoding gene regulation. Nature 499, 172-177, doi:10.1038/nature12311 (2013)) such as ARGONAUTE proteins (AGO) (Meister, G. Argonaute proteins: functional insights and emerging roles. Nature reviews. Genetics 14, 447-459, doi:10.1038/nrg3462 (2013)), PUM2, QKI (Hafner, M. et al. Transcriptome-wide identification of RNA-binding protein and microRNA target sites by PAR-CLIP. Cell 141, 129-141, doi:10.1016/j.cell.2010.03.009 (2010)), and snoRNP proteins (Granneman, S., Kudla, G., Petfalski, E. & Tollervey, D. Identification of protein binding sites on U3 snoRNA and pre-rRNA by UV cross-linking and high-throughput analysis of cDNAs. Proceedings of the National Academy of Sciences of the United States of America 106, 9613-9618, doi:10.1073/pnas.0901997106 (2009)). Despite recent advances such as PAR-CLIP (Hafner, M. et al. Transcriptome-wide identification of RNA-binding protein and microRNA target sites by PAR-CLIP. Cell 141, 129-141, doi:10.1016/j.cell.2010.03.009 (2010)), HITS-CLIP (Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009)), and CLASH (Helwak, A., Kudla, G., Dudnakova, T. & Tollervey, D. Mapping the human miRNA interactome by CLASH reveals frequent noncanonical binding. Cell 153, 654-665, doi:10.1016/j.cell.2013.03.043 (2013); Kudla, G., Granneman, S., Hahn, D., Beggs, J. D. & Tollervey, D. Cross-linking, ligation, and sequencing of hybrids reveals RNA-RNA interactions in yeast. Proceedings of the National Academy of Sciences of the United States of America 108, 10010-10015, doi:10.1073/pnas.1017386108 (2011)), it remains a formidable challenge to map all protein-assisted RNA-RNA interactions.

In each of these three approaches, only the interactions mediated by one RNA-binding protein can be analyzed per experiment. Additionally, each experiment requires either a protein-specific antibody (HITS-CLIP or PAR-CLIP) or stable expression of a tagged protein in transformed cell lines (CLASH). Furthermore, any two RNAs that co-appeared in either HITS-CLIP or PAR-CLIP could have resulted from the independent attachment of either RNA to different copies of the targeted protein. For example, suppose 10 AGO proteins were present in a cell, each of which was bound by a different RNA; these 10 RNAs would be identified as interacting from AGO HITS-CLIP. Therefore, HITS-CLIP and PAR-CLIP inferred RNA-RNA interactions did not necessarily occur in the cells analyzed.

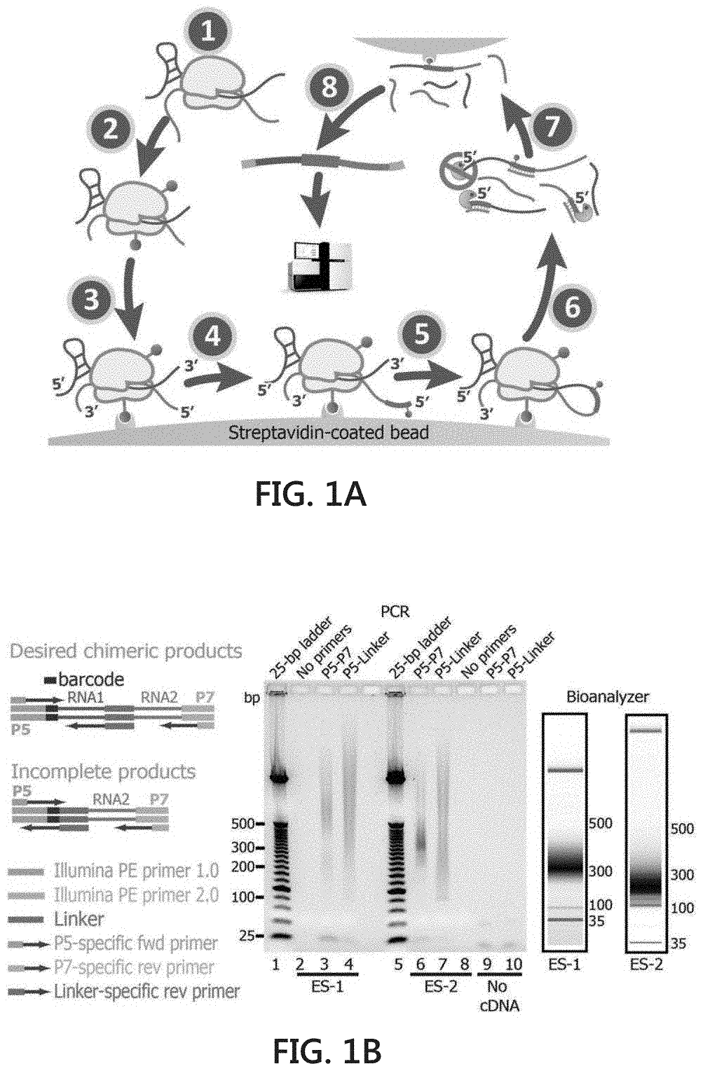

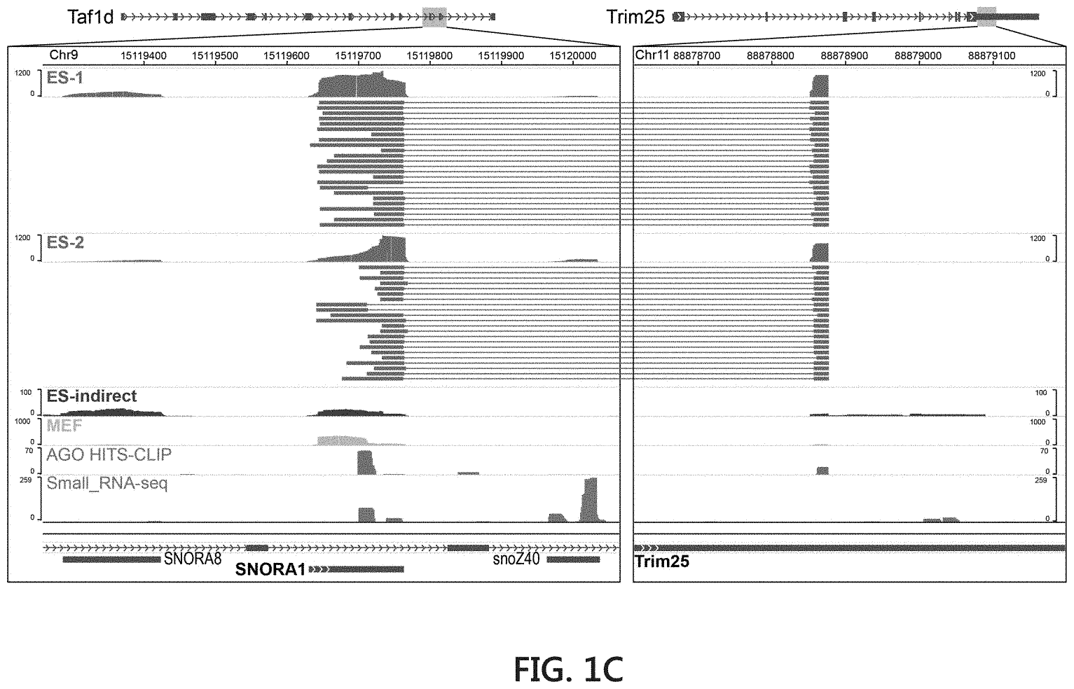

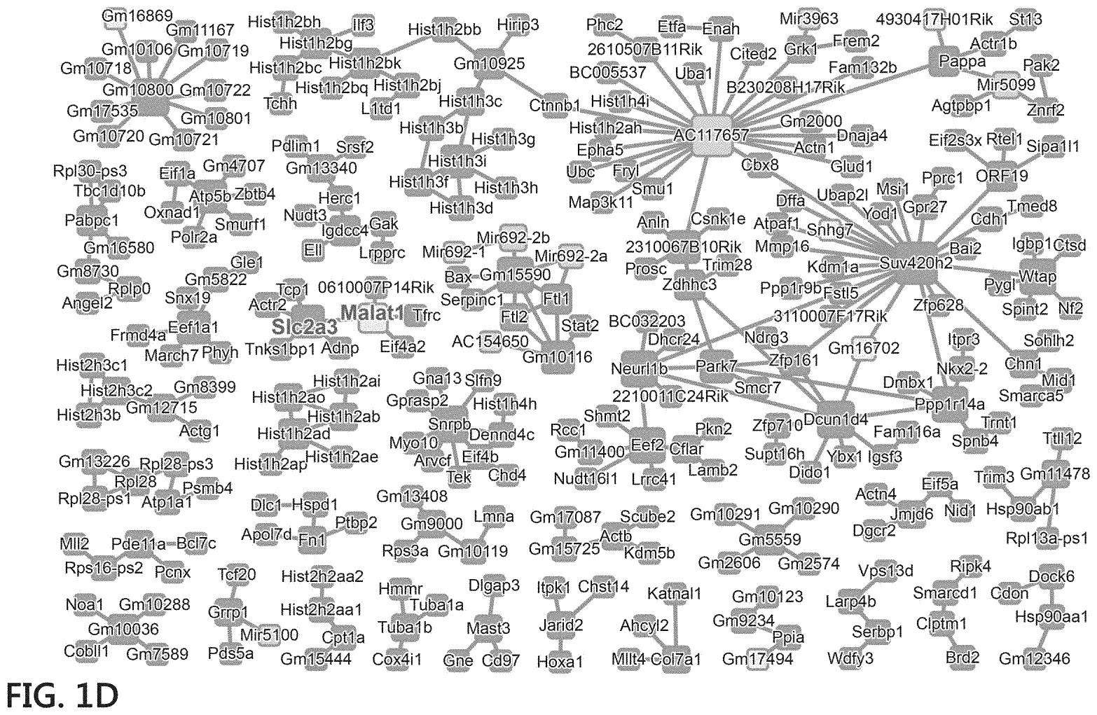

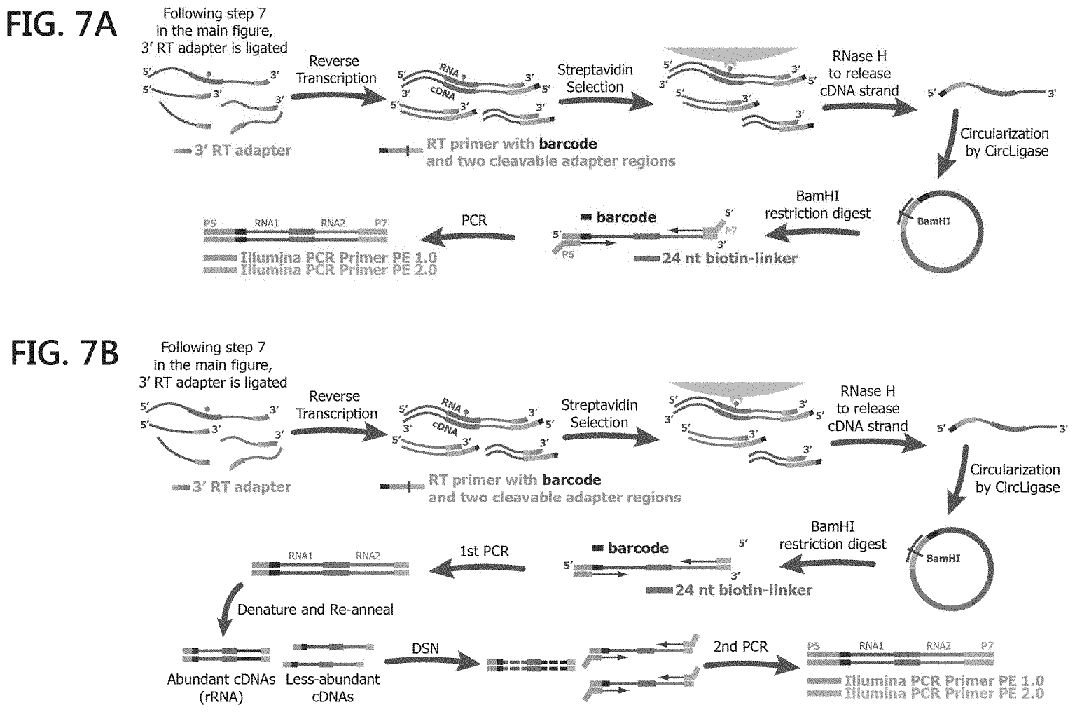

In an exemplary embodiment described herein, an RNA Hi-C method was developed to detect protein-assisted RNA-RNA interactions in vivo. In this procedure, RNA is cross-linked with its bound proteins then ligated to a biotinylated RNA linker such that the RNAs, RNA1 and RNA2, are co-bound by the same protein forming a chimeric RNA of the form RNA1-Linker-RNA2. These linker-containing chimeric RNAs are isolated using streptavidin coated magnetic beads and subjected to pair-end sequencing (Methods, FIG. 1A, FIGS. 7A to 7B). Thus, each non-redundant pair-end read reflects a molecular interaction.

The RNA Hi-C method offers several advantages for mapping RNA-RNA interactions. First, only the RNAs brought together by the same protein molecule are captured, overcoming the drawback in HITS-CLIP where different RNAs would be considered as interacting when they are independently bound to different copies of the same protein. Second, the use of a biotinylated linker as a selection marker circumvents the requirement for a protein-specific antibody or the need to express a tagged protein. This allows for an unbiased mapping of the RNA interactome. As described in the art, other methods can only work with one RNA-binding protein at a time. Thus this method leads to the surprising effect of working efficiently with more than one RNA-binding protein at a time. Third, false positives that result from RNAs ligating randomly to other nearby RNAs are minimized by performing the RNA ligation step on streptavidin beads in extremely dilute conditions. Fourth, the RNA linker provides a clear boundary delineating sequencing reads that span across the ligation site, thus avoiding ambiguities in mapping the sequencing reads. Fifth, RNA Hi-C directly analyzes the endogenous cellular condition without introducing any exogenous nucleotides (Hafner, M. et al. Transcriptome-wide identification of RNA-binding protein and microRNA target sites by PAR-CLIP. Cell 141, 129-141, doi:10.1016/j.cell.2010.03.009 (2010); Lal, A. et al. Capture of microRNA-bound mRNAs identifies the tumor suppressor miR-34a as a regulator of growth factor signaling. PLoS genetics 7, e1002363, doi:10.1371/journal.pgen.1002363 (2011); Baigude, H., Ahsanullah, Li, Z., Zhou, Y. & Rana, T. M. miR-TRAP: a benchtop chemical biology strategy to identify microRNA targets. Angew Chem Int Ed Engl 51, 5880-5883, doi:10.1002/anie.201201512 (2012)) or protein-coding genes (Helwak, A., Kudla, G., Dudnakova, T. & Tollervey, D. Mapping the human miRNA interactome by CLASH reveals frequent noncanonical binding. Cell 153, 654-665, doi:10.1016/j.cell.2013.03.043 (2013)), prior to cross-linking. Sixth, potential PCR amplification biases are removed by attaching a random 6 nucleotide barcode to each chimeric RNA before PCR amplification and subsequently counting completely overlapping sequencing reads with identical barcodes only once (Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009); Loeb, G. B. et al. Transcriptome-wide miR-155 binding map reveals widespread noncanonical microRNA targeting. Molecular cell 48, 760-770, doi:10.1016/j.molcel.2012.10.002 (2012); Wang, Z. et al. iCLIP predicts the dual splicing effects of TIA-RNA interactions. PLoS biology 8, e1000530, doi:10.1371/journal.pbio.1000530 (2010); Konig, J. et al. iCLIP reveals the function of hnRNP particles in splicing at individual nucleotide resolution. Nature structural & molecular biology 17, 909-915, doi:10.1038/nsmb.1838 (2010)).

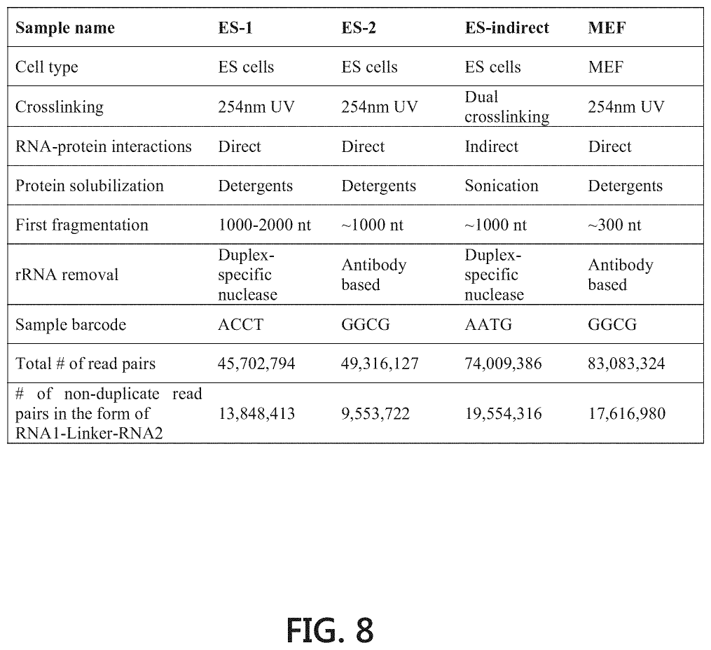

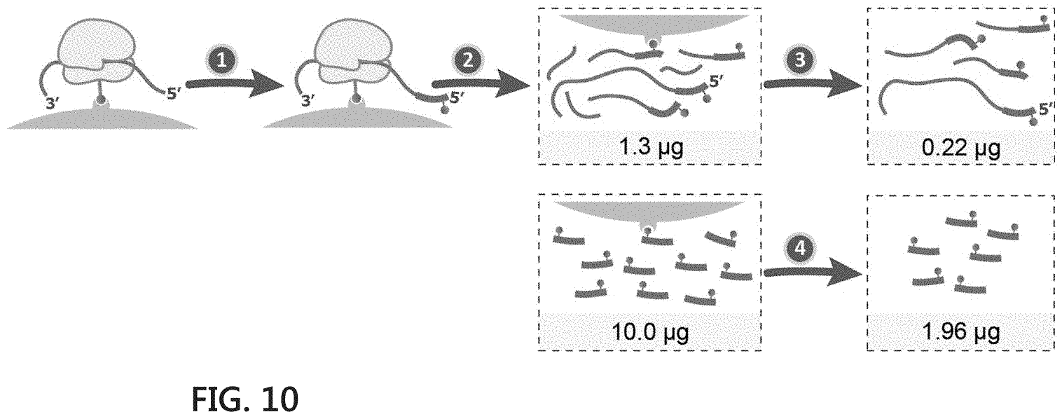

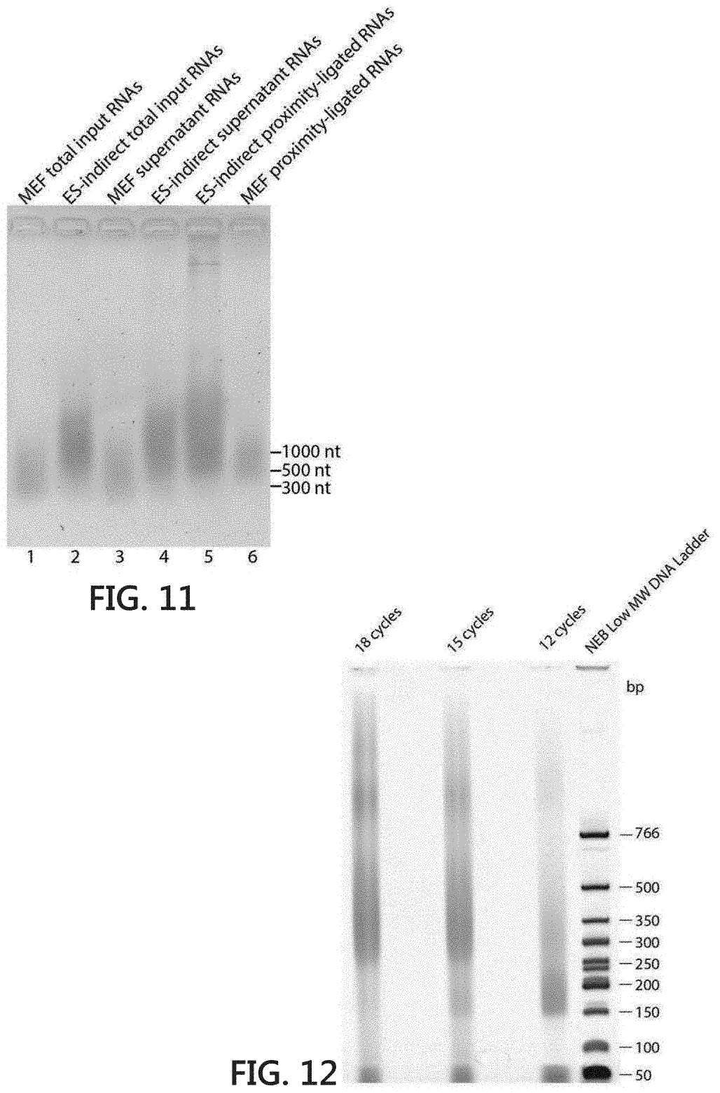

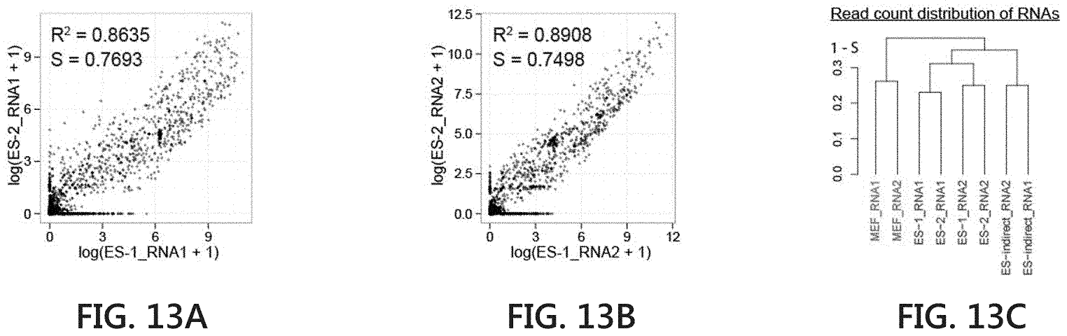

In an exemplary embodiment, two independent RNA Hi-C assays were carried out on mouse embryonic stem (ES) cells with minor technical differences (FIGS. 8, 9A to 9D, 10, 11 and 12), which were designated as ES-1 and ES-2. To control for the RNAs assembled by large protein complexes (Zhao, J. et al. Genome-wide identification of polycomb-associated RNAs by RIP-seq. Molecular cell 40, 939-953, doi:10.1016/j.molcel.2010.12.011 (2010)) or cell organelles instead of a single protein, an RNA Hi-C library was generated using two cross-link agents (formaldehyde and EGS) that form covalent bonds between both nucleotides and proteins and between proteins (ES-indirect) (Nowak, D. E., Tian, B. & Brasier, A. R. Two-step cross-linking method for identification of NF-kappaB gene network by chromatin immunoprecipitation. BioTechniques 39, 715-725 (2005); Zeng, P. Y., Vakoc, C. R., Chen, Z. C., Blobel, G. A. & Berger, S. L. In vivo dual cross-linking for identification of indirect DNA-associated proteins by chromatin immunoprecipitation. BioTechniques 41, 694, 696, 698 (2006)). Another library was produced from mouse embryonic fibroblasts (MEF), offering one more dataset for bioinformatic quality assessment (FIGS. 13A to 13C). It was confirmed that each library contained RNA constructs of the desired form (RNA1-Linker-RNA2) and lengths (FIG. 1B). Each library was sequenced to yield, on average, 47.3 million pair-end reads, among which approximately 15.1 million non-redundant pair-end reads represented the desired chimeric form (FIG. 1C).

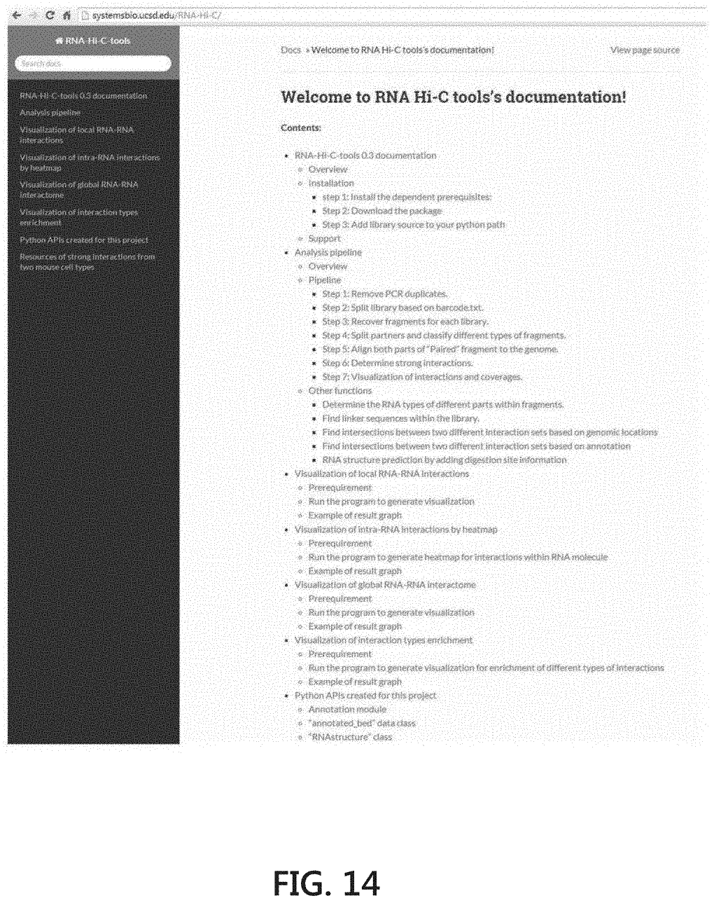

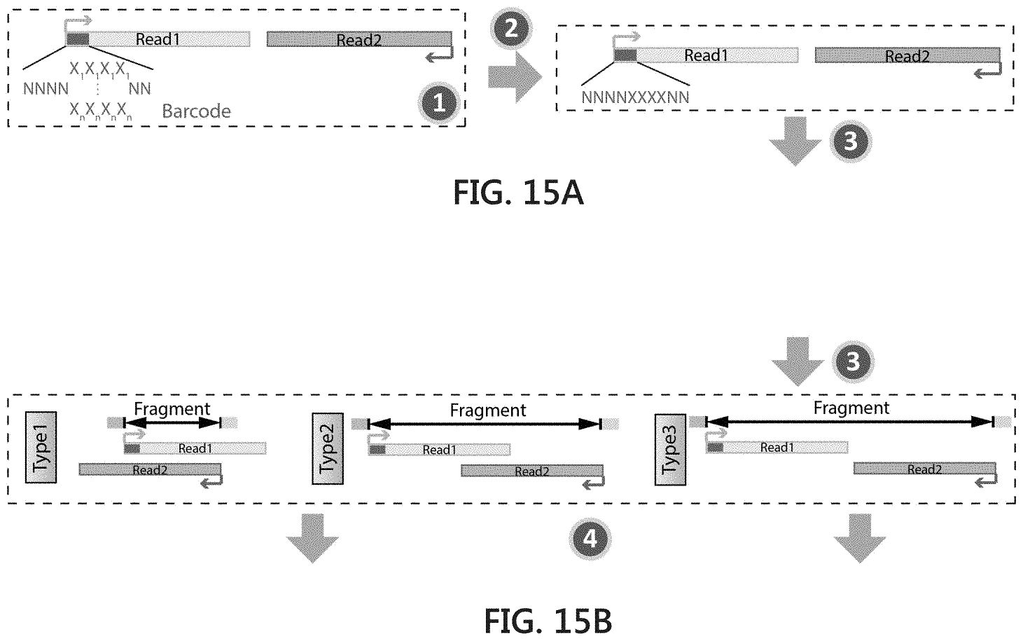

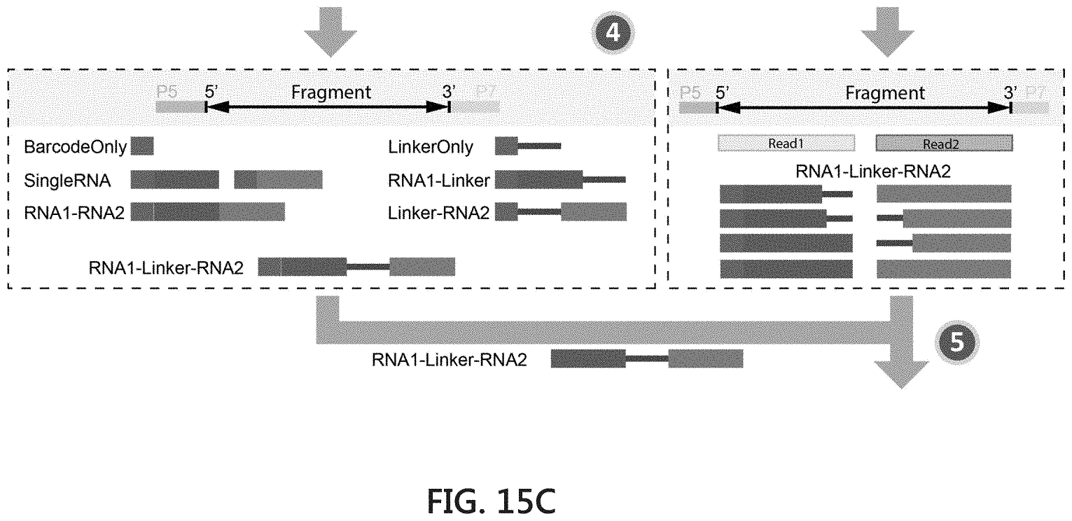

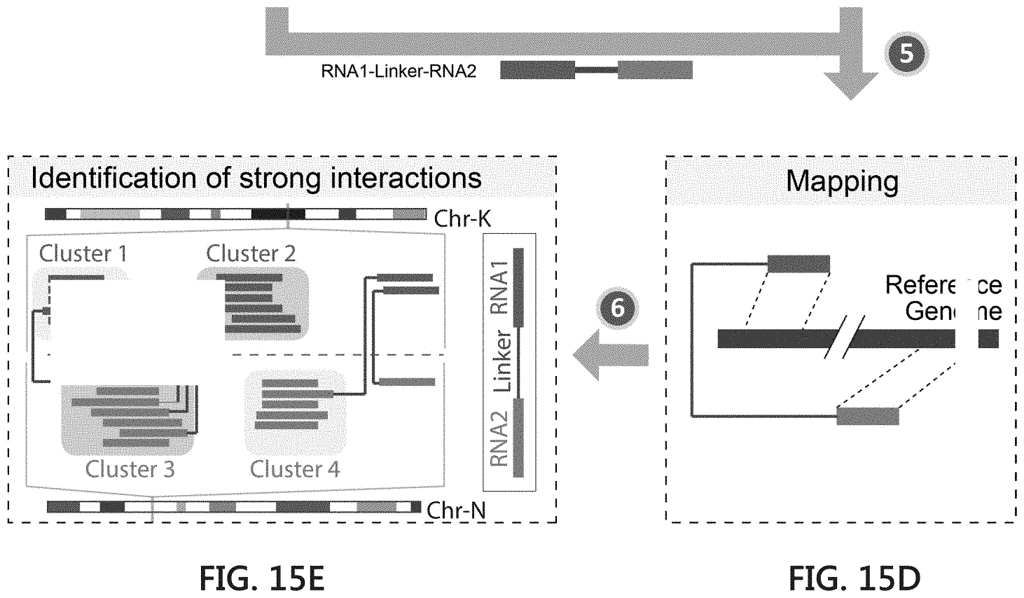

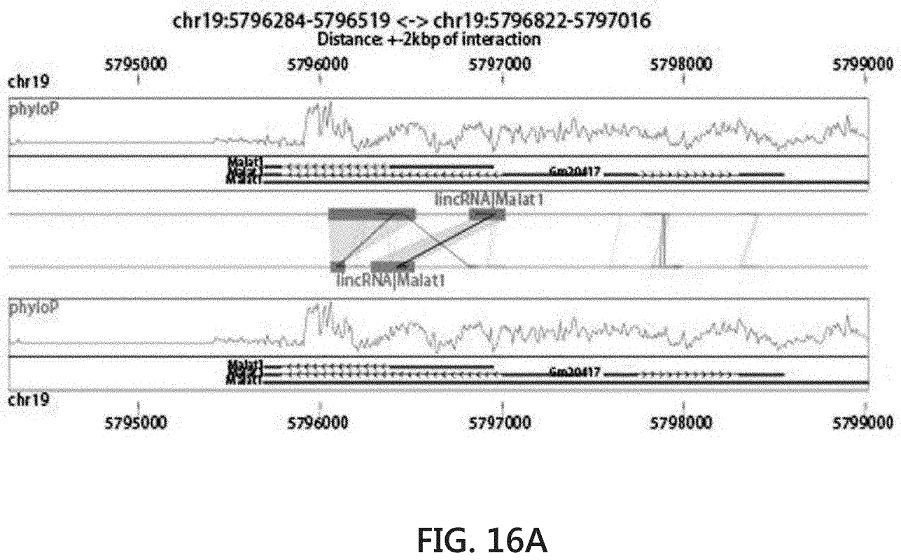

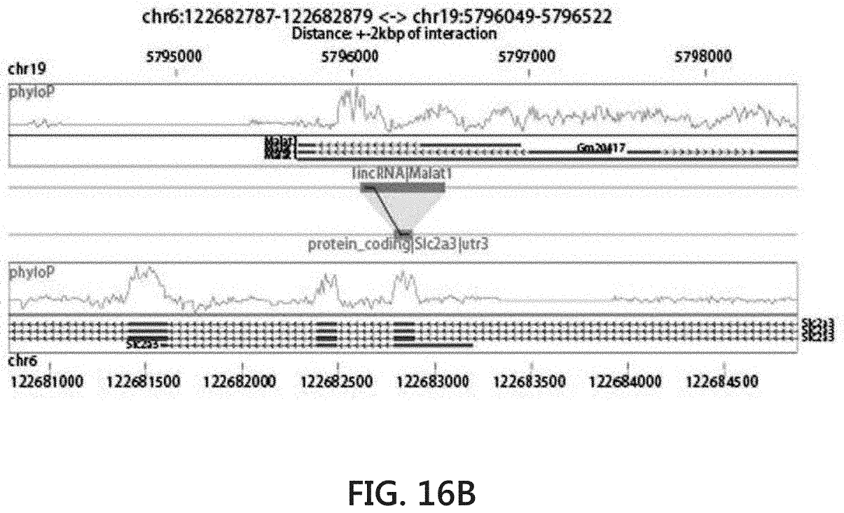

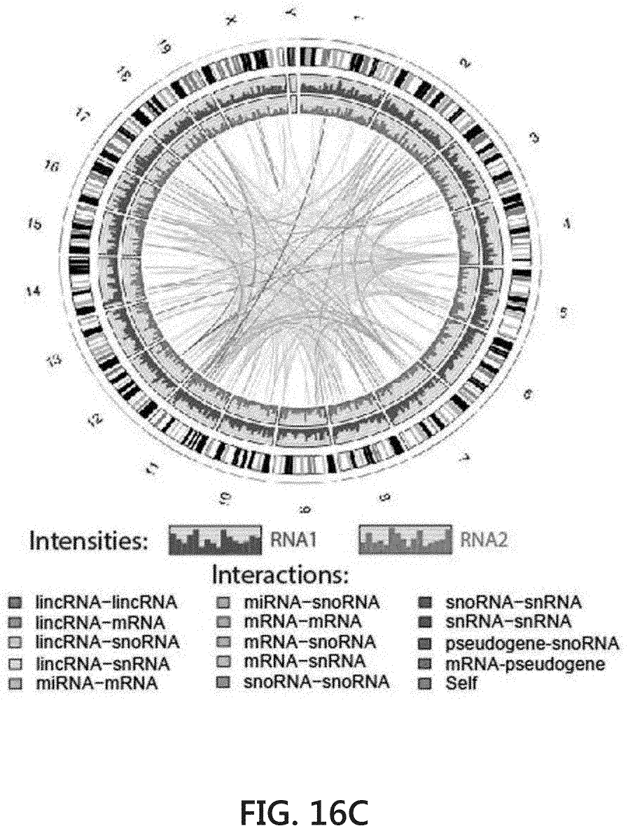

A set of bioinformatic tools was created (RNA-HiC-tools) to analyze and visualize RNA Hi-C data (FIGS. 14 and 15A to 15E). RNA-HiC-tools automated the analysis steps, including removing PCR duplicates, splitting multiplexed samples, identifying the linker sequence, splitting junction reads, calling interacting RNAs, performing statistical assessments, categorizing RNA interaction types, calling interacting sites, and analyzing RNA structure (Methods). It also provides visualization tools for both the RNA interactome and the proximal sites within an RNA (FIGS. 16A to 16C).

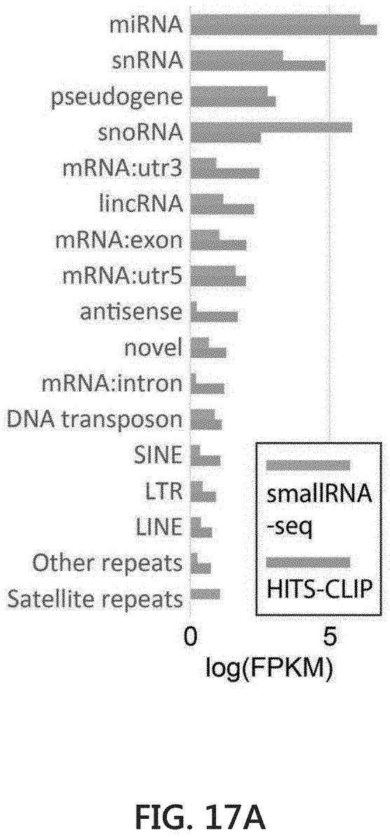

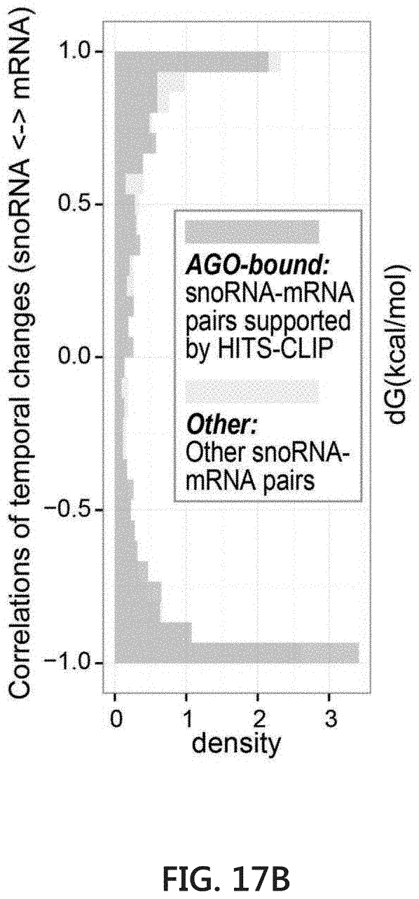

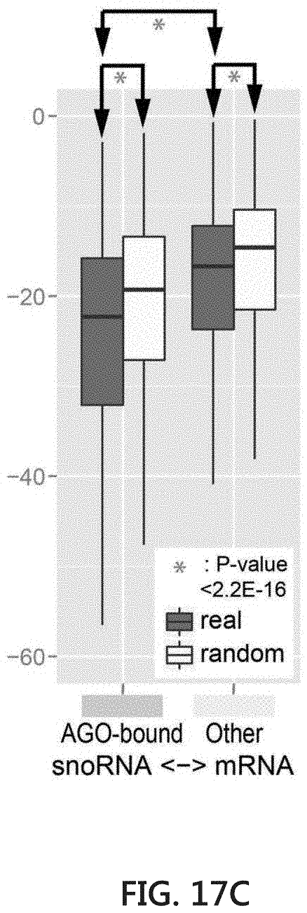

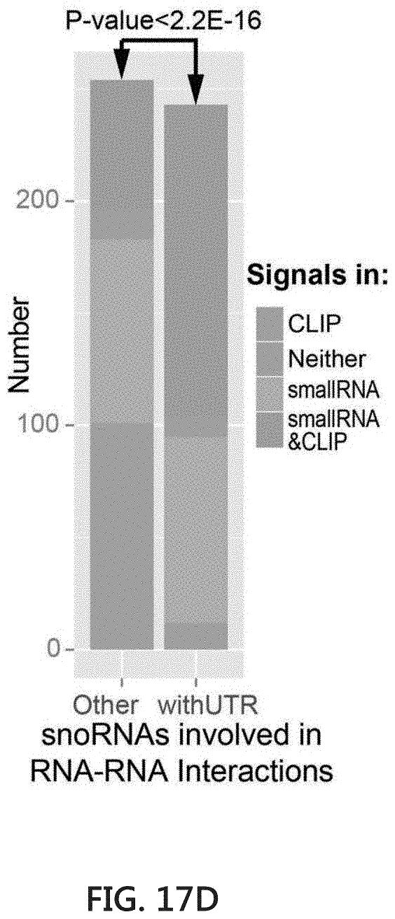

The four RNA Hi-C libraries were compared. ES-1 and ES-2 were most similar judged by correlations of FPKMs (separately calculated for the read fragments on the left and the right sides of the linker), followed by ES-indirect, and then MEF (FIG. 13A to 13C). The interacting RNA pairs identified from ES-1 and those from ES-2 exhibited strong overlaps (p-value <10⁻³⁵, permutation test). The interactions identified in MEF did not exhibit significant overlaps with those in either of the ES samples (p-value for each overlap=1, permutation tests). For example, an interaction between the 3′ UTR of Trim25 RNA and small nucleolar RNA (snoRNA) Snora1 was supported by 24 and 22 pair-end reads in ES-1 and ES-2 samples, respectively, but was not detected in ES-indirect or MEF libraries (FIG. 1C). Including Snora1, as many as 172 snoRNAs that were identified as interacting with mRNAs was supported by AGO HITS-CLIP (FIG. 1C) and small RNA sequencing data (Yu, P. et al. Spatiotemporal clustering of the epigenome reveals rules of dynamic gene regulation. Genome research 23, 352-364, doi:10.1101/gr.144949.112 (2013)) (FIG. 1C, FIGS. 17A to 17D, 18 and 19A to 19C), suggesting that most of the expressed snoRNA genes were enzymatically processed into miRNA-like small RNAs and interacted with mRNAs in RISC complex (Ender, C. et al. A human snoRNA with microRNA-like functions. Molecular cell 32, 519-528, doi:10.1016/j.molcel.2008.10.017 (2008); Brameier, M., Herwig, A., Reinhardt, R., Walter, L. & Gruber, J. Human box C/D snoRNAs with miRNA like functions: expanding the range of regulatory RNAs. Nucleic Acids Res 39, 675-686, doi:10.1093/nar/gkq776 (2011)) (Text S1).

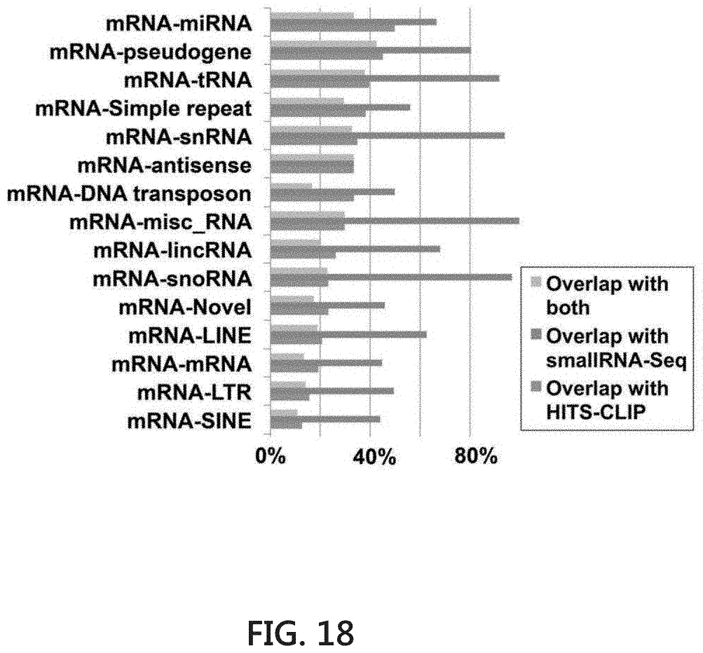

It was then desired to know whether other RNAs could experience a similar process to miRNA biogenesis and also interact with mRNAs. To do so, the RNA Hi-C identified interacting RNAs were intersected with those found by small RNA sequencing (smallRNA-seq) and those bond to the AGO protein (HITS-CLIP) in ES cells (S. W. Chi, J. B. Zang, A. Mele, R. B. Darnell, Argonaute HITS-CLIP decodes microRNA-mRNA interaction maps. Nature 460, 479 (Jul. 23, 2009)). The smallRNA-seq selectively sequenced, “miRNAs and other small RNAs that have a 3′ hydroxyl group resulting from enzymatic cleavage by Dicer or other RNA processing enzymes” (IIlumina, “TruSeq® Samll RNA Sample Preparation Guide” (2014)). Besides miRNA, other RNA types including snoRNA, pseudogene RNA, mRNA UTRs also contributed to the small RNA pool, and were attached to AGO (FIG. 17A). Moreover, large portions of RNA Hi-C identified interacting RNA pairs co-appeared in AGO HITS-CLIP data (FIG. 18). This data suggest there are non-miRNAs that are digested by DICER or other RNA processing enzymes and are incorporated into the RISC complex.

To elucidate what types of non-miRNA genes were most likely to undergo miRNA-like biogenesis, the RNA Hi-C identified RNA-RNA interactions were subjected to the following filters:
1. the interaction involves one mRNA (dubbed target) and one other RNA (source RNA);
2. the source RNA is processed into small RNA by enzymatic cleavage (FPKM>0 in smallRNA-seq);
3. both the target and the source RNAs appear in AGO HITS-CLIP (FPKM>0 for both RNAs);
4. the RNA Hi-C identified interaction sites on the source and the target RNAs exhibit strong base pairing (p-value <0.05, Wilcoxon signed-rank test comparing the binding energies between the RNA1 and RNA2 sequences of every pair-end read to the binding energies of randomly shuffled nucleotide sequences).

A total of 302 RNA-RNA interactions passed these filters. The majority (79%) of the source RNAs in these interactions were snoRNAs (Table 2). The snoRNAs were therefore prioritized for functional analysis.

TABLE 2
miRNA-like RNAs. The RNA Hi-C identified RNA-RNA interactions were filtered
by (1) involving an mRNA (dubbed target) and one other RNA (dubbed source RNA),
(2) the source RNA was present in smallRNA-seq, (3) both the target and the
source RNAs appeared in AGO HITS-CLIP, (4) the RNA Hi-C identified interaction
sites on the source and the target RNAs exhibit strong base pairing. Column
2 lists the number of interaction sites that satisfied the criteria 1-3. Column
3 lists the number of interaction sites that satisfied criteria 1-4. Column
4 lists the number of interactions that satisfied criteria 1-4.

| | # of | # of interaction | | |
|---|---|---|---|---|
| | interaction | sites on source | # of | |
| | sites on | RNAs with base | interactions | |
| Source | source | pairing to | with base | Interaction sites on the source |
| RNA | RNAs | target mRNAs | pairing | RNAs (mm9) |
| snoRNA | 172 | 83 | 226 | http://systemsbio.ucsd.edu/RNA-Hi- |
| | | | | C/Data/OtherRNAs_as_miRNA.htm |
| snRNA | 22 | 8 | 16 | http://systemsbio.ucsd.edu/RNA-Hi- |
| | | | | C/Data/OtherRNAs_as_miRNA.htm |
| mRNA | 68 | 8 | 8 | chr18: 48207763-48207972 |
| | | | | chr17: 13184946-13185035 |
| | | | | chr6: 67233894-67234046 |
| | | | | chr9: 64039312-64039420 |
| | | | | chr11: 69730265-69730433 |
| | | | | chr17: 6121531-6121797 |
| | | | | chr13: 45011825-45011869 |
| | | | | chr6: 115757003-115757184 |
| LINE | 7 | 1 | 8 | chr2: 90235277-90235370 |
| Misc_RNA | 4 | 2 | 4 | chr2: 6997218-6997460, |
| | | | | chr4: 43505643-43505934 |
| SINE | 3 | 1 | 2 | chr6: 128748868-128748976 |
| | | | | chr13: 107911768-107911832 |
| Pseudogene | 13 | 1 | 1 | chr11: 86444105-86444271 |
| LTR | 5 | 1 | 1 | chr18: 10052120-10052158 |

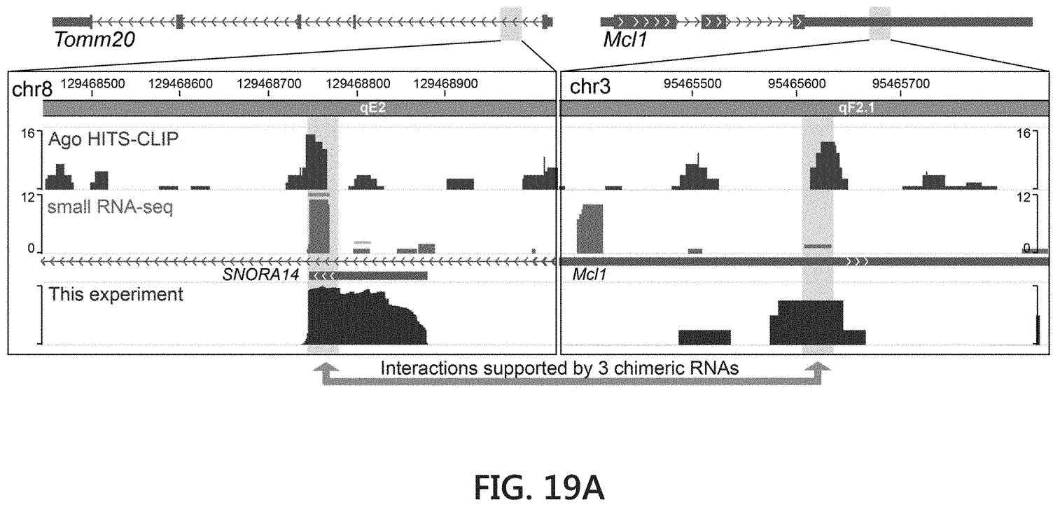

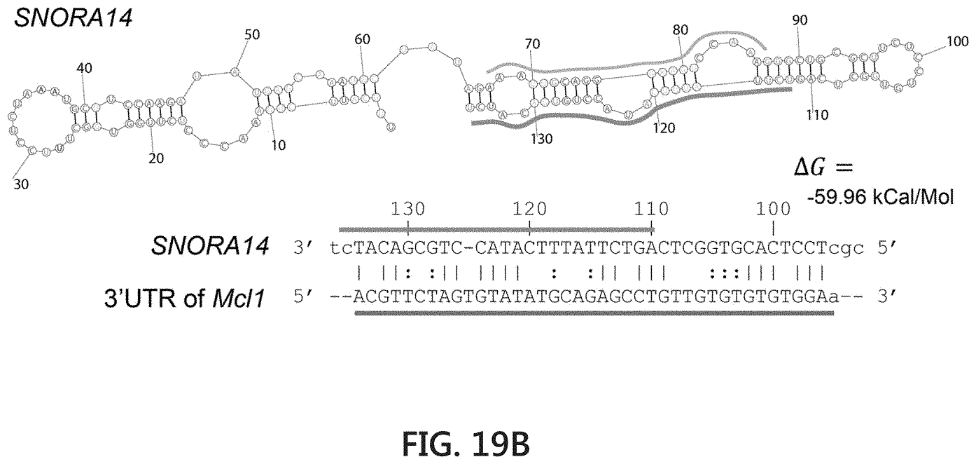

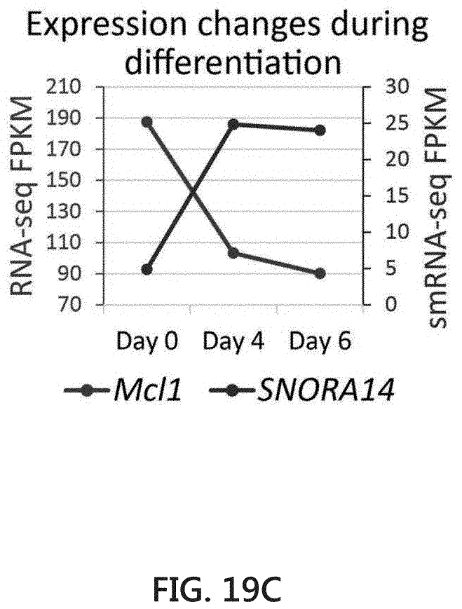

It was hypothesized that a large number of snoRNAs were enzymatically processed into miRNA-like short RNAs and interact with mRNAs. This hypothesis was supported by 919 RNA Hi-C identified snoRNA-mRNA interactions where both the mRNA and the snoRNA were bound by AGO. Furthermore, AGO bound snoRNAs and their interacting mRNAs exhibited anti-correlated expression changes during guided differentiation of ES cells toward mesendoderm (P. Yu et al., Spatiotemporal clustering of the epigenome reveals rules of dynamic gene regulation. Genome research 23, 352 (February, 2013)) (FIG. 17B). Additionally, AGO bound snoRNAs and their target mRNAs exhibited stronger base pairing than that without AGO binding (FIG. 17C). Finally, the small RNAs processed from snoRNAs referentially interacted with the UTR regions of mRNAs. Out of the 497 snoRNAs involved in RNA-RNA interactions, 243 interacted with UTR regions, among which 223 (92%) were detected in smallRNA-seq, suggesting the experience of an enzymatic cut (FIG. 17D). In comparison, the other 254 snoRNAs interacting with non-UTR regions contained fewer (55%) small RNAs. Besides, two times more UTR-interacting sno-siRNAs were AGO bound than the non-UTR interacting snoRNAs (p-value <2.2⁻¹⁶, Chi-square test). For example, Snora14 RNA targeted the 3′ UTR of Mcl1 mRNA (FIG. 19A). The interacting site on Snora14 RNA (110-135 nt) precisely overlapped with the enzymatically processed small RNA as well as the AGO bound region. The enzymatically processed portion of Snora14 RNA is located completely on one side of a hairpin loop (FIG. 19B), and exhibits a strong binding affinity (−60 kCal/mol) to the target site on Mcl1 UTR. The expression of the processed Snora14 RNA was anticorrelated with that of Mcl1 mRNA (FIG. 19C). Taken together, this data suggest a large number of small interfering RNAs originated from snoRNA genes, which interact with more than 900 mRNAs in ES cells.

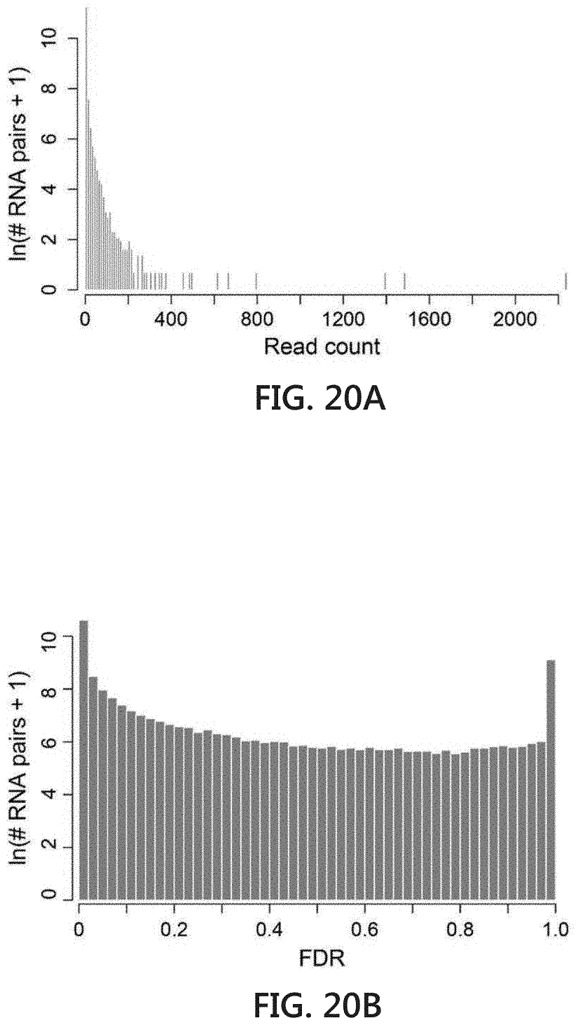

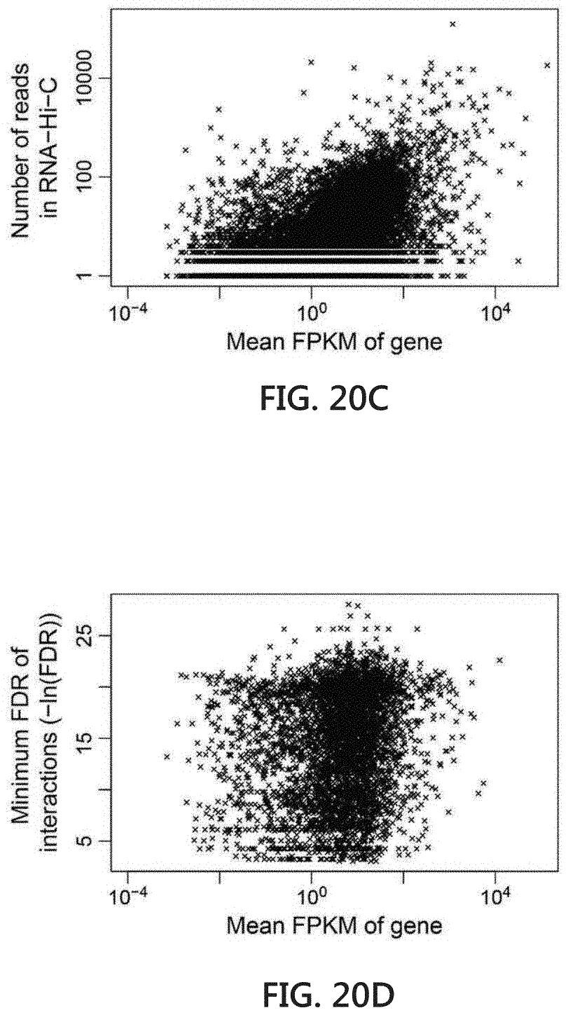

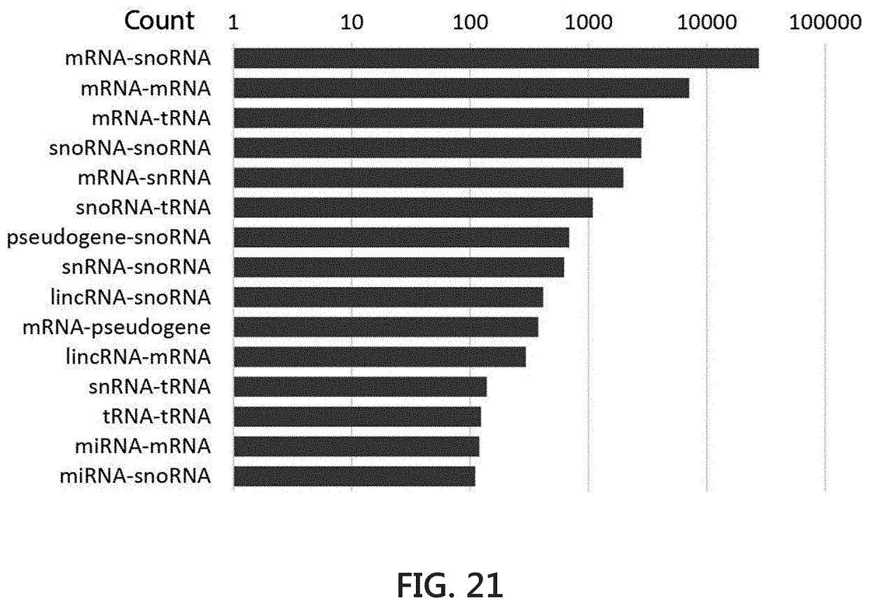

The ES-1 and ES-2 libraries were merged to infer the RNA interactome in ES cells. This data included 4.54 million non-duplicated pair-end reads that were unambiguously split into two RNA fragments with both fragments uniquely mapping to the genome (mm9). 46,780 inter-RNA interactions were identified (FDR<0.05, Fisher's exact test) (FIGS. 20A to 20D). mRNA-snoRNA interactions were the most abundant type, although thousands of mRNA-mRNA and hundreds of lincRNA-mRNA, pseudogeneRNA-mRNA, miRNA-mRNA interactions were also detected (FIG. 21). This is probably the first RNA interactome described in any organism. Thus, the simulation suggested approximately 66% sensitivity and 93% specificity for the entire experimental and analysis procedure (Text S2).

### Simulation Analysis of RNA Hi-C

1.1 Data synthesis. In order to estimate the sensitivity and specificity of RNA Hi-C, including its experimental and computational procedures, a simulation analysis was carried out. 1,000,000 pair-end reads by computationally mimicking the data generation process were simulated. The parameters used for the simulation were derived from real data. The simulated data generation process is as follows.

For each pair-end read (2×100 bases):
1. A sample barcode from the four sample barcodes with equal probabilities and concatenate it with a 6 nt random barcode was chosen (as in FIG. 15A).
2. Assigned this pair-end read to a type of cDNAs from the list of [linkerOnly, NoLinker, RNA1-linker, linker-RNA2, RNA1-linker-RNA2] with probability [0.1, 0.3, 0.1, 0.3, 0.2], respectively (as in FIG. 15C).
3. If this read-pair was assigned to a linker-containing type, randomly choose 1 or 2 linkers with equal probability. It is noted that a small percentage of linker-containing read-pairs contained 2 linkers; the use of equal probability was a conservative choice for estimating worst cases.
4. Generate the sequences for the RNA1 and the RNA2 parts, according to the cDNA type determined in Step 2. For both RNA1 and RNA2,
   a. simulate its length from l˜Unif (15,150),
   b. choose an RNA type from [“miRNA”, “mRNA”, “lincRNA”, “snoRNA”, “snRNA”, “tRNA”] based on the following probabilities:
      i. if length l<50, use [0.2,0.2,0.1,0.2,0.2,0.1],
      ii. otherwise, use [0.05,0.4,0.2,0.2,0.1,0.05];
   c. randomly choose an RNA according to the sampled RNA type from Ensembl (release 67, mouse NCBIM37),
   d. randomly take a sequence segment with length l from the chosen RNA.
5. Concatenate the barcodes, linker, and RNA fragments generated from Steps 1, 3, 4, producing a synthetic cDNA sequence.
6. If the synthetic cDNA in Step 5 is 100 bp or longer, take the 100 bases from the two ends of the synthetic cDNA in forward and reverse strands respectively.
7. If the synthetic cDNA in Step 5 is shorter than 100 bp, assign its forward and reverse strands as the forward and the reverse reads, and concatenate P5 and P7 primer sequences to the two reads.
8. Simulate sequencing errors with a rate of 0.01 on each base (N. J. Loman et al., Performance comparison of benchtop high-throughput sequencing platforms. Nature biotechnology 30, 434 (May, 2012)).

Steps 1-5 simulated a cDNA sequence according the experimental procedure, and steps 6-8 simulated a pair-end read based on this cDNA sequence. The simulated interacting RNA pairs, as well as the cDNA type and the length of each part (RNA1, linker, and RNA2, if applicable) were kept for comparison with the computational predictions.

1.2. Evaluation of intermediate and final results. The synthetic data was used to evaluate the sensitivities and specificities of two intermediate analysis steps, as well as the final predictions.

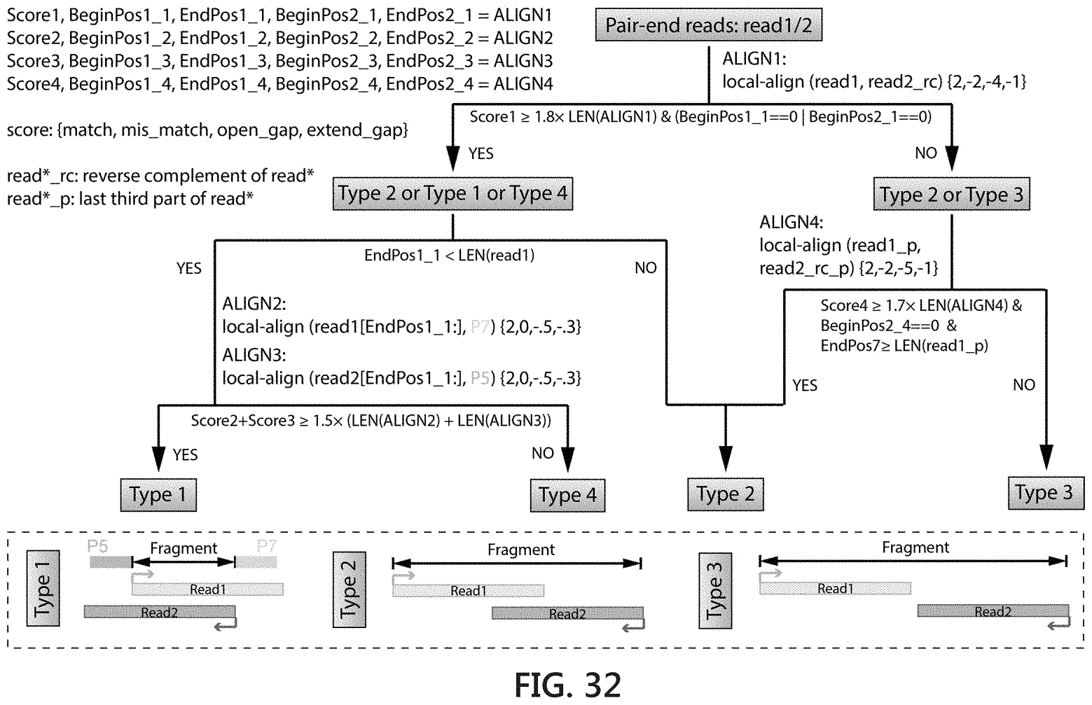

First, the predicted cDNA lengths were compared (output of Step 3 of RNA-HiC-Tools) to the actual lengths (Table 3). This step “3. Recovering the cDNAs in the sequencing library” assigns each cDNA into four types with respect to their lengths, namely Type 1 (<100 bp); Type 2 (100-200 bp); Type 3 (>200 bp); Type 4 (unknown) (FIG. 32). The algorithm achieved high sensitivity and specificity for predicting each type. Only very few (0.58%) of the cDNAs shorter than 200 bp were predicted to be longer than 200 bp. These errors were due to a small overlap (typically between 0 and 5 bps) of the forward and the reverse reads, which were not detected by the local alignment.

TABLE 3
A comparison of the predicted and true
cDNA length ranges. The counts of
cDNAs of each type (Columns 1-4) are
compared to their true types (rows).

| | Predicted | | | | | |
|---|---|---|---|---|---|---|
| True | Type 1 | Type 2 | Type 3 | Type 4 | Sensitivity | Specificity |
| Type 1 | 312,411 | 24 | — | — | 99.99% | 99.97% |
| Type 2 | 65 | 480,835 | 5,750 | 898 | 98.62% | 99.73% |
| Type 3 | 126 | 1,322 | 197,716 | 853 | 98.84% | 99.28% |

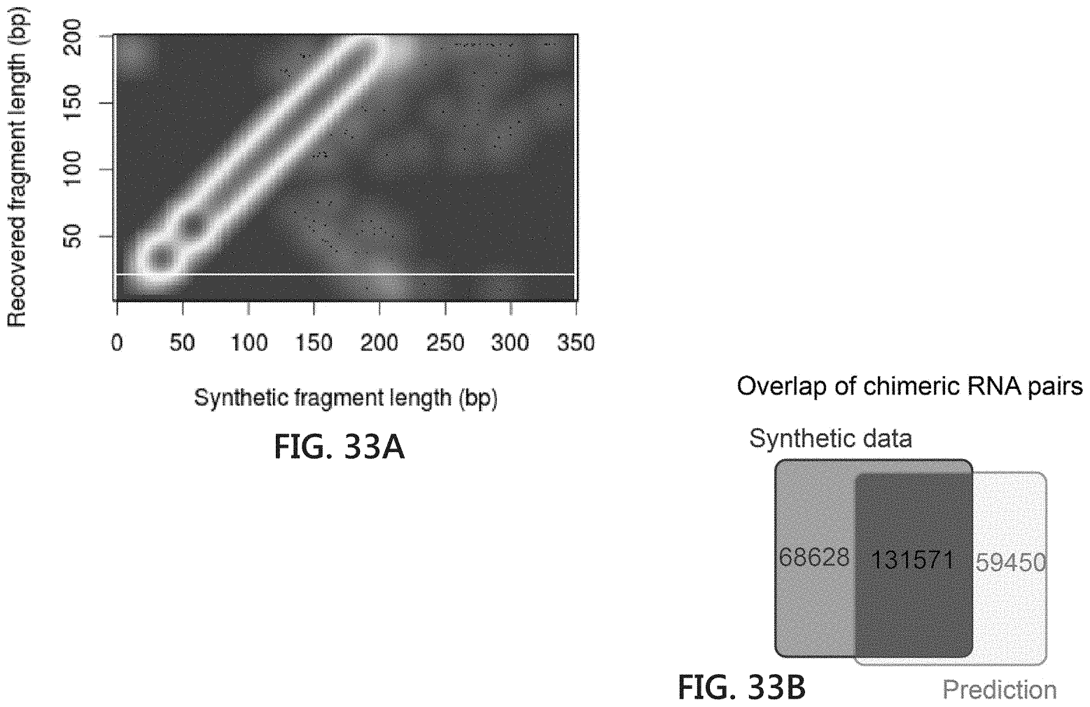

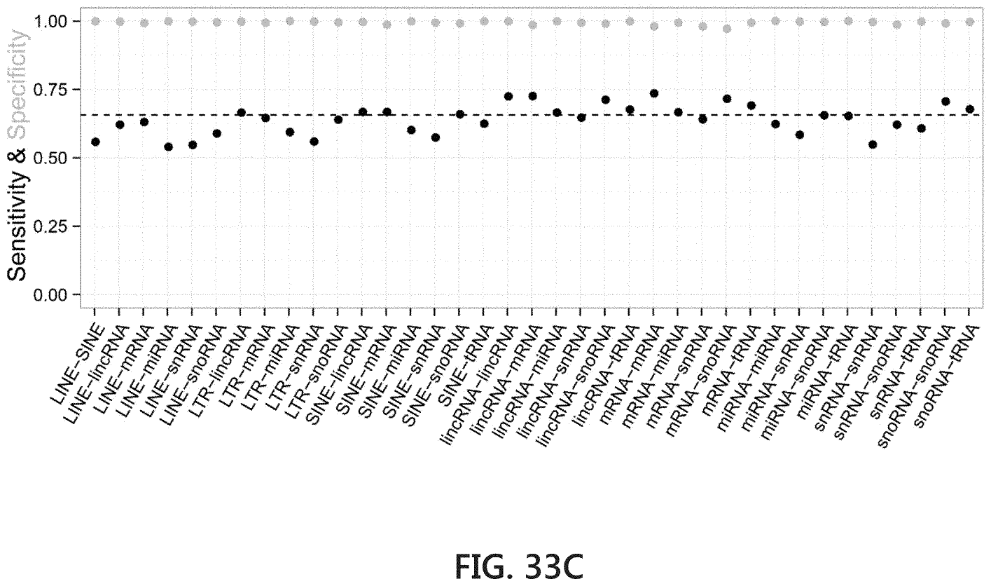

When the predicted length was shorter than 200 bp (Types 1 and 2), the exact length could be predicted. In these cases, the predicted lengths often precisely matched the lengths of the simulated cDNAs (FIG. 33A).

Next, the predicted chimeric configuration of each cDNA was compared (output of Step 4 of RNA-HiC-Tools) to the synthesized configuration. In Step “4. Parsing the chimeric cDNAs”, the algorithm assigned the cDNAs into five categories, based on the presence of the linker sequence. The algorithm reached 99.89% sensitivity and 95.82% specificity for the cDNAs in the “RNA1-linker-RNA2” form (Table 4).

TABLE 4
A comparison of the predicted and true cDNA configurations.
The counts of cDNAs of the predicted configurations (columns)
are compared to their true configurations (rows).

| | Predicted | | | | |
|---|---|---|---|---|---|
| | | | | | R1- |
| | | | | Linker- | linker- |
| True | NoLinker | LinkerOnly | R1-linker | R2 | R2 |
| NoLinker | 266,554 | 10 | — | — | 33,402 |
| LinkerOnly | — | 100,230 | — | — | — |
| R1-linker | 24 | 25 | 100,267 | — | — |
| Linker-R2 | 50 | 58 | — | 299,180 | — |
| R1-linker-R2 | 57 | 116 | 24 | 22 | 199,981 |

Lastly, the predicted and the simulated RNA-RNA interactions were compared. The simulated dataset contained 200,200 chimeric RNA pairs, among which 131,571 pairs of RNAs were detected (sensitivity=65.72%, specificity=92.57%, FIG. 33C). The sensitivity and specificity for interactions of each type of RNAs was also separately calculated (FIG. 33C). Regardless of the types of participating RNAs, the method showed few false positives (specificity ≥90%). Interactions that did not involve transposon RNA or snRNA exhibited fewer false negatives than those that did. This was due to the repetitive nature of transposon and snRNA sequences. The worst cases involved LINE RNAs, where sensitivities dropped to 52%. It was conservatively estimated that about a half of the interactions involving transposon RNAs could have been missed by this procedure. It was estimated that about ⅔ to ¾ of the interactions that do not involve transposon RNAs would have been identified.

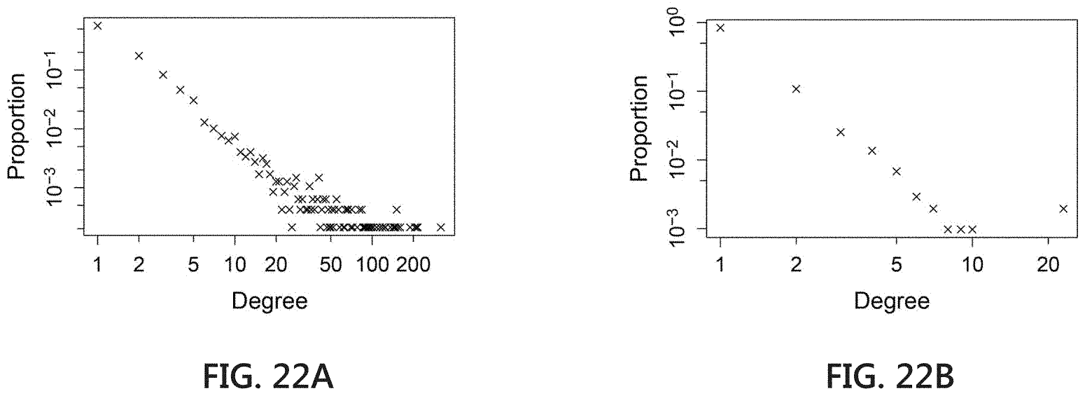

The number of interacting partners per RNA was strongly unbalanced. The ES cell RNA interactome was a scale-free network, with a degree distribution that conformed to power law P(k)˜k^{-γ}, γ=3) (FIG. 22A) (Barabasi, A. L. & Oltvai, Z. N. Network biology: understanding the cell's functional organization. Nature reviews. Genetics 5, 101-113, doi:10.1038/nrg1272 (2004)). To see whether the scale-free property was driven by a small number of highly connected snoRNAs, snRNAs, and tRNAs, they were removed them from the network. The interactions composed only of mRNAs, lincRNAs, miRNAs, pseudogene RNAs, and antisense RNAs remained scale-free (FIG. 22B). A number of mRNAs, pseudogene RNAs, and lincRNAs emerged as hubs (nodes with large numbers of connections, FIG. 1D). The largest mRNA hub was Suv420h2, which interacted with 21 mRNAs and 2 lincRNAs. The largest lincRNA hub was Malat1, which interacted with 4 mRNAs, including an mRNA hub of Slc2a3.

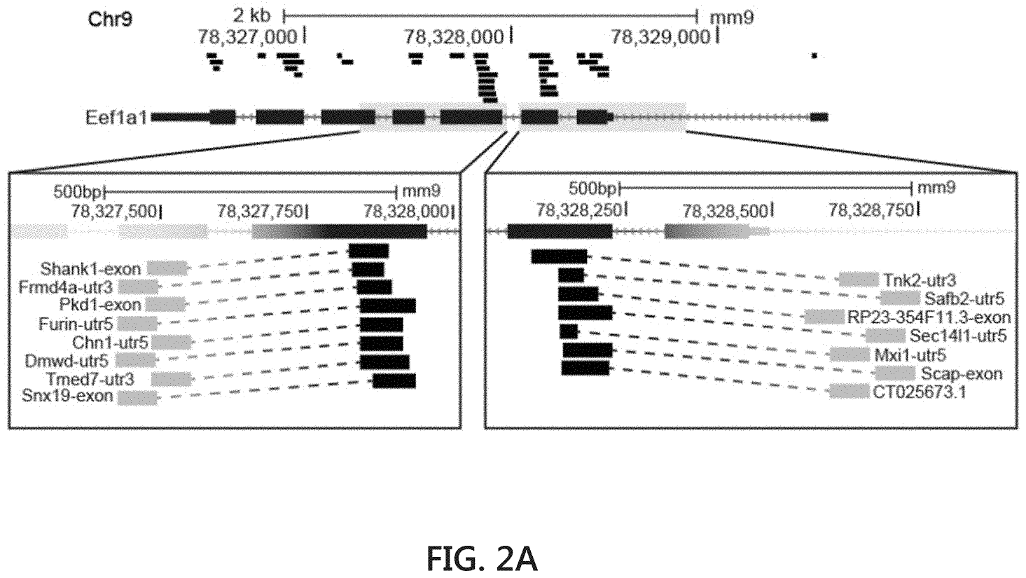

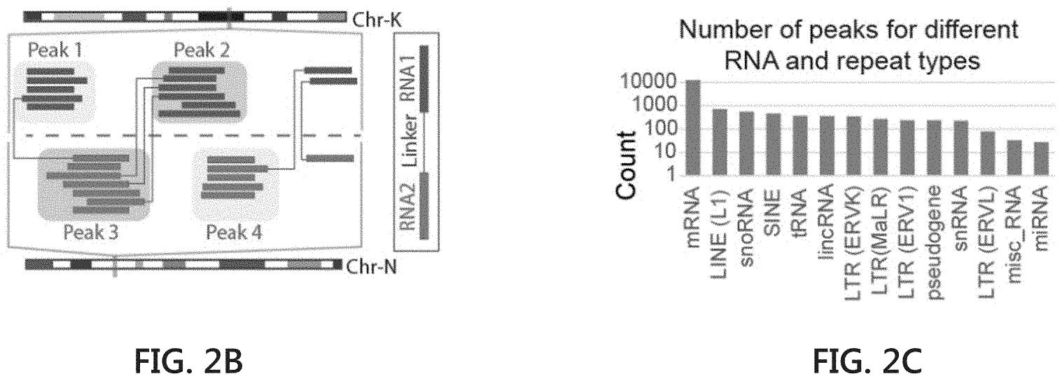

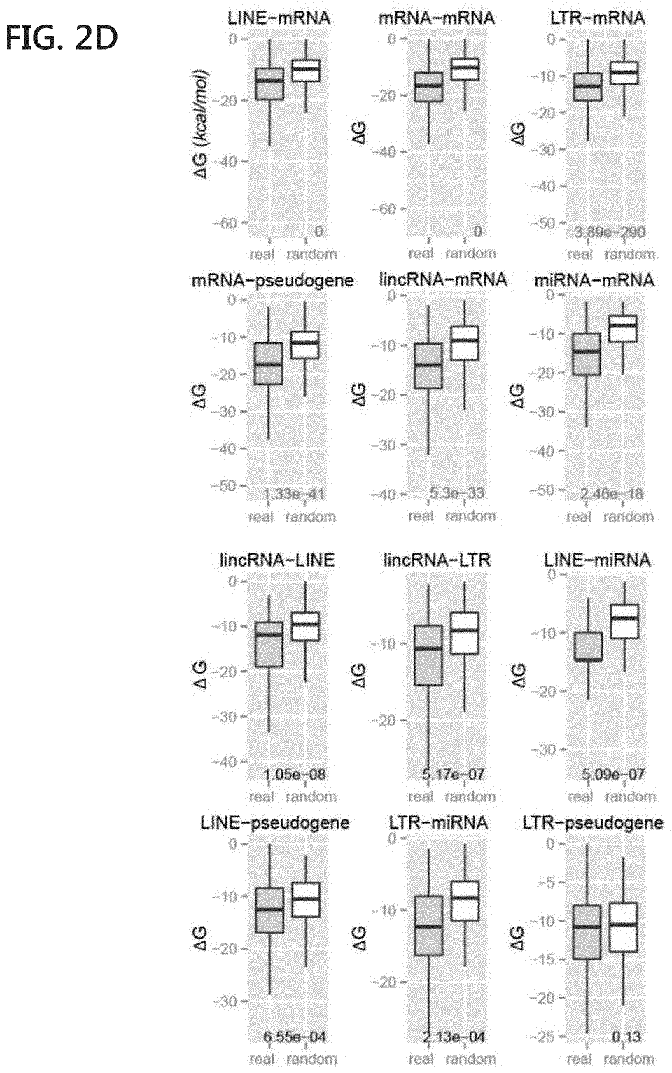

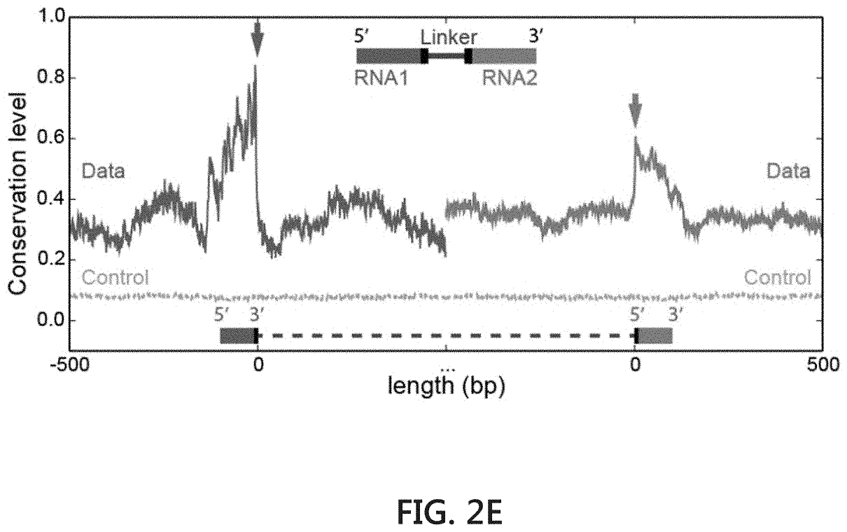

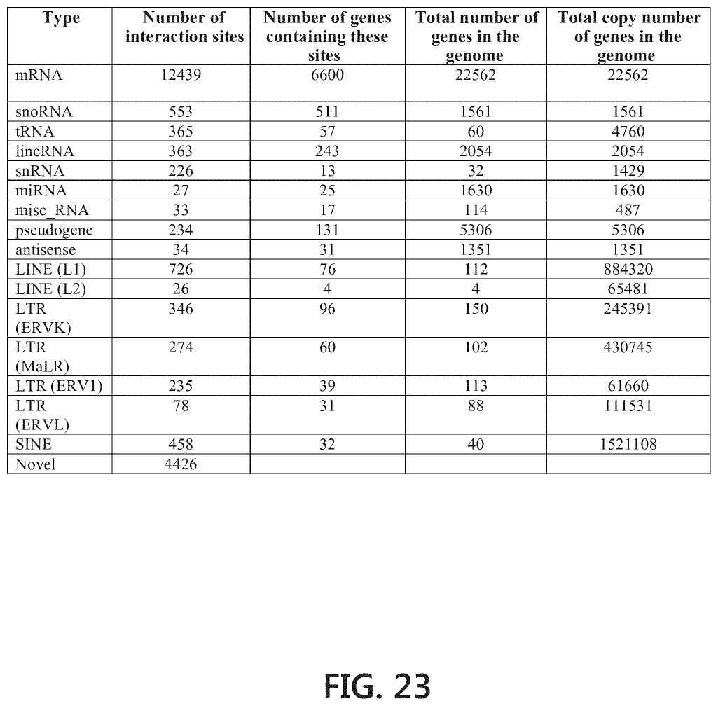

The majority (83.05%) of the interacting RNAs exhibited overlapping RNA Hi-C reads (FIG. 2A), suggesting interactions were often concentrated at specific segments of an RNA. “Peaks” of overlapping read fragments were identified and termed “interaction sites” (FIG. 2B). Interaction sites appeared not only on miRNAs (the entire mature miRNA), mRNAs, lincRNAs, but also on pseudogene and transposon RNAs (FIG. 2C). Over 2000 interaction sites were harbored in L1, SINE, ERVK, MaLR, and ERV1 transposon RNAs (FIG. 23), indicative of their frequent interactions with other RNAs (Shalgi, R., Pilpel, Y. & Oren, M. Repression of transposable-elements—a microRNA anti-cancer defense mechanism? Trends in genetics: TIG 26, 253-259, doi:10.1016/j.tig.2010.03.006 (2010); Yuan, Z., Sun, X., Liu, H. & Xie, J. MicroRNA genes derived from repetitive elements and expanded by segmental duplication events in mammalian genomes. PloS one 6, e17666, doi:10.1371/journal.pone.0017666 (2011)).

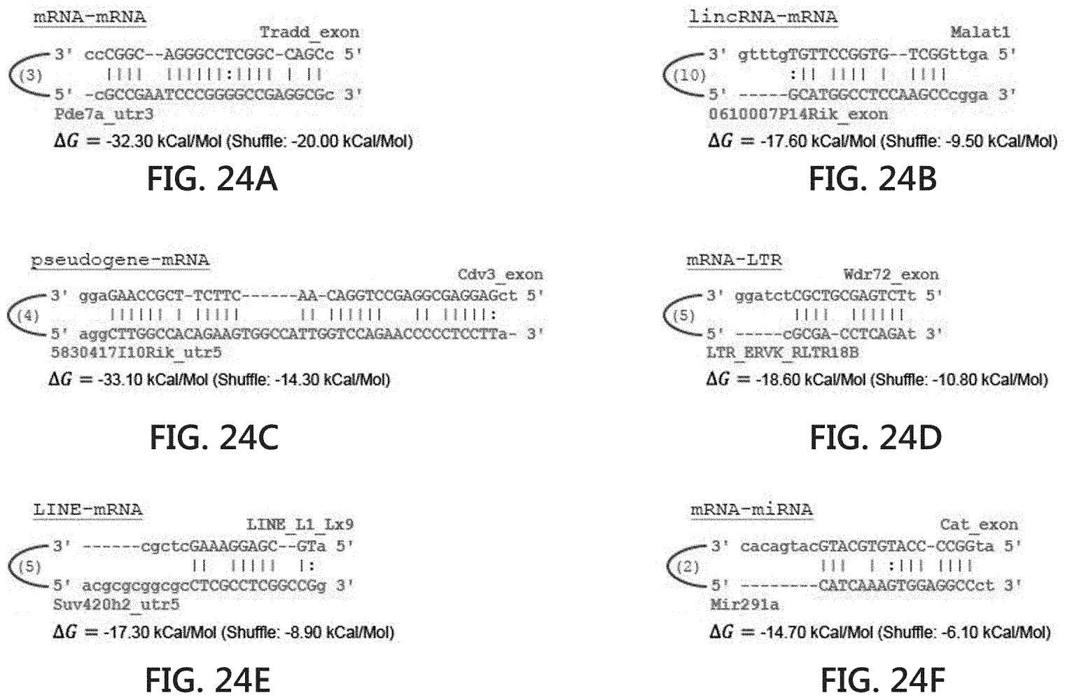

It was postulated whether base complementation is utilized by different types of RNA-RNA interactions. The hybridization energy of a pair of interacting RNAs was estimated by the average hybridization energy of the pairs of ligated fragments (RNA1, RNA2) (Bellaousov, S., Reuter, J. S., Seetin, M. G. & Mathews, D. H. RNAstructure: web servers for RNA secondary structure prediction and analysis. Nucleic Acids Res 41, W471-W474, doi:Doi 10.1093/Nar/Gkt290 (2013)), and was compared to the hybridization energy of control RNAs generated by random shuffling of the bases. Complementary bases were preferred in nearly all types of RNA-RNA interactions, and were most pronounced in transposonRNA-mRNA, mRNA-mRNA, pseudogeneRNA-mRNA, lincRNA-mRNA, miRNA-mRNA interactions (p-values <2.4⁻¹⁸), but was not observed in LTR-pseudogeneRNA interactions (FIG. 2D, FIGS. 24A to 24F). This data suggests a new mechanism, where base pairing facilitates sequence-specific posttranscriptional regulation in long RNAs.

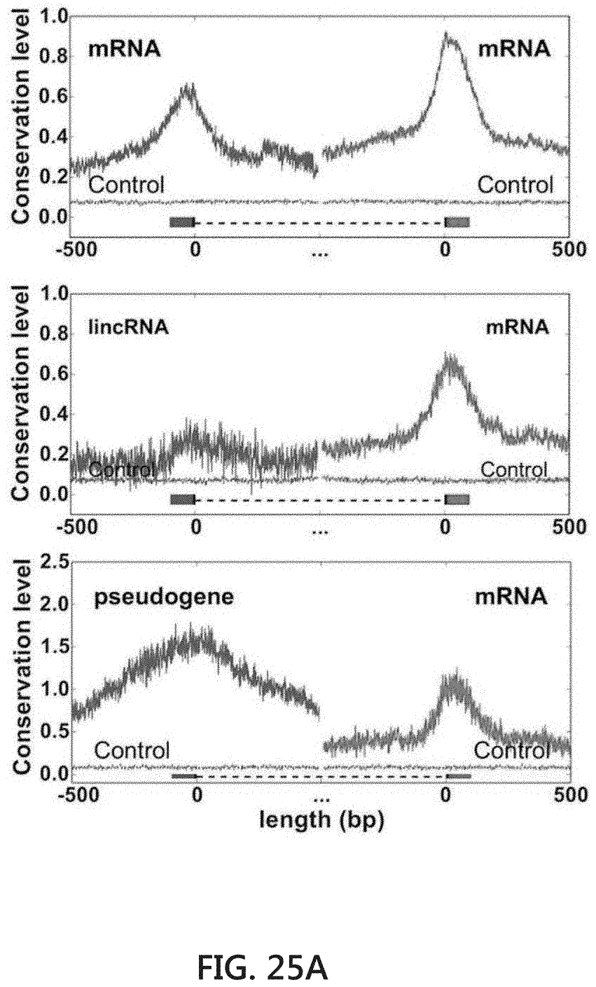

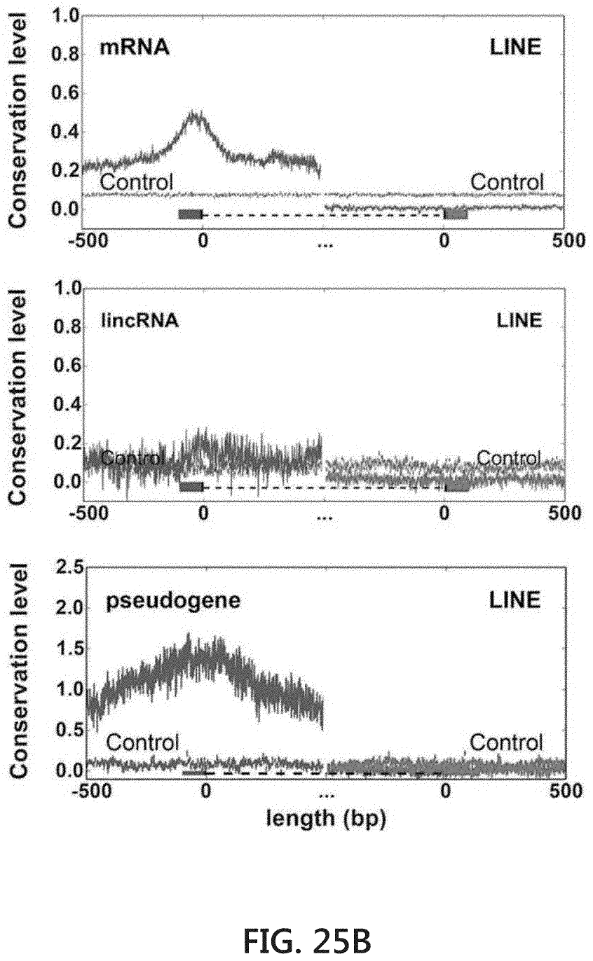

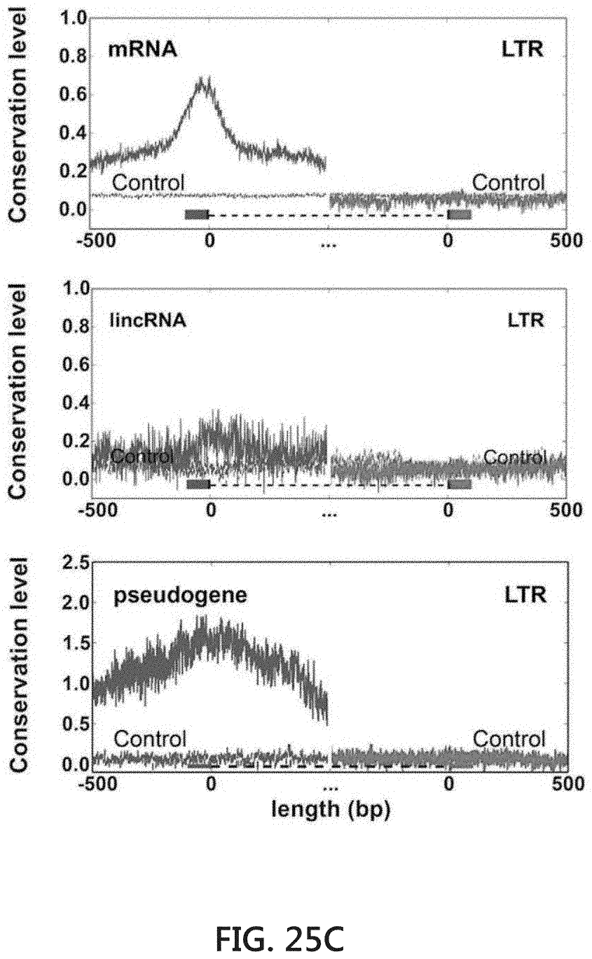

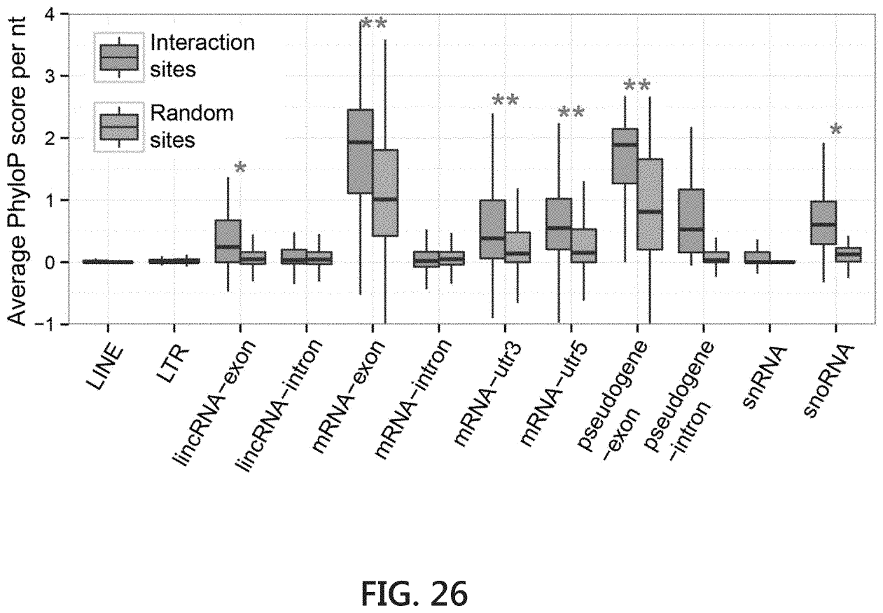

If these RNA-RNA interactions are sequence-specific, the RNA interaction sites should be under selective pressure. It was found that the interspecies conservation levels (Cooper, G. M. et al. Distribution and intensity of constraint in mammalian genomic sequence. Genome research 15, 901-913, doi:10.1101/gr.3577405 (2005)) are strongly increased at the interaction sites, and the peak of conservation precisely pinpointed the junction of the two RNA fragments (FIG. 2D). When interacting with lincRNAs, pseudogene RNAs, transposon RNAs, or other mRNAs, the interaction sites on mRNAs were more conserved than the rest of the transcripts (FIGS. 25A to 25C). The interactions sites on lincRNAs and pseudogene RNAs exhibited increased conservation in lincRNAs-mRNA, pseudogeneRNA-mRNA, and pseudogeneRNA-transposonRNA interactions (FIGS. 25A to 25C). The increased conservation on interaction sites was not due to exon-intron boundaries (FIG. 26). Taken together, base complementation is wide-spread in the interactions of long RNAs, and is evolutionarily selected. This suggests a new type of regulatory information encoded in the genome.

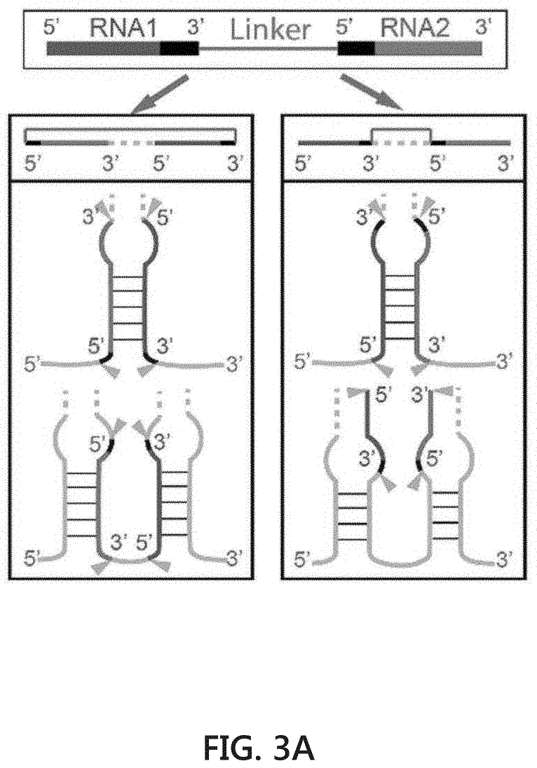

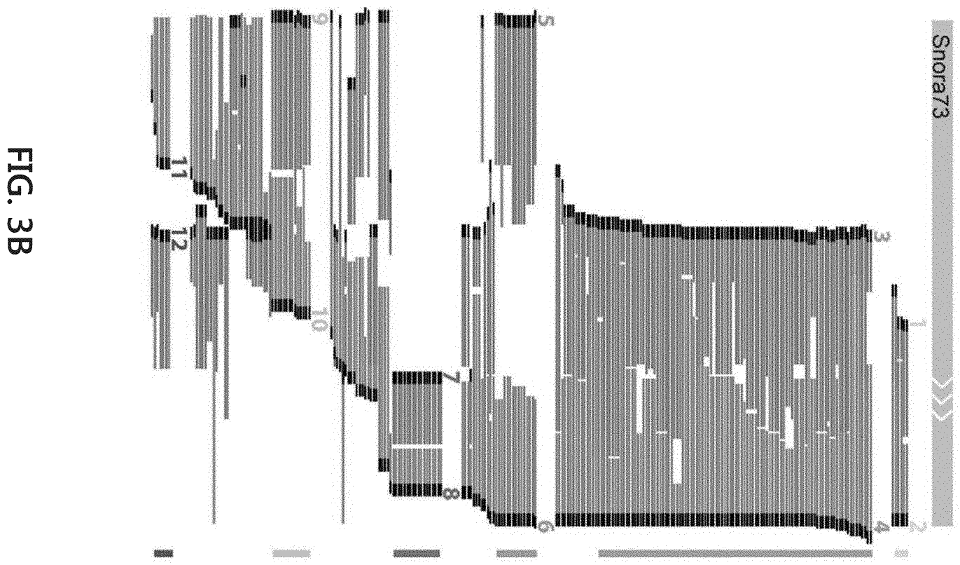

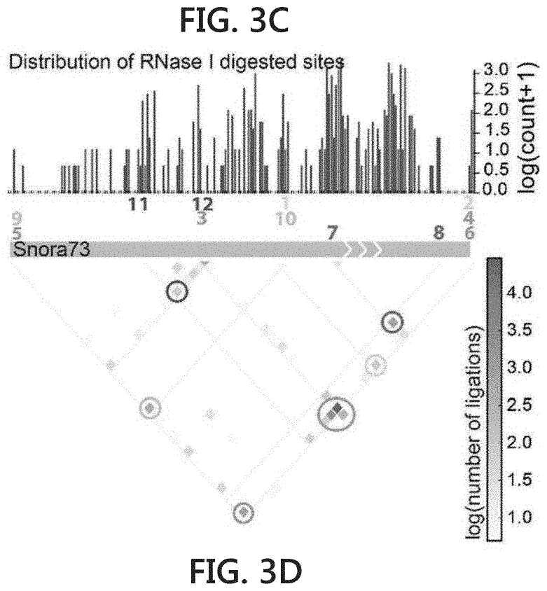

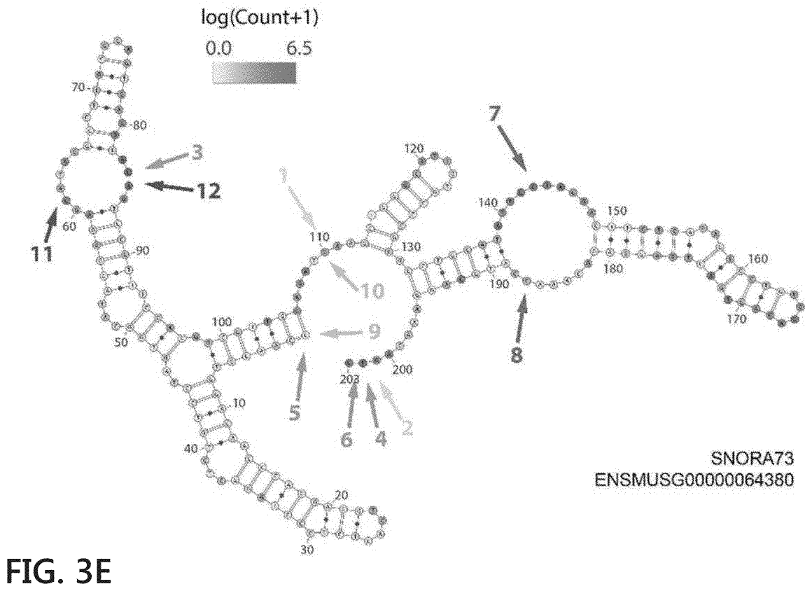

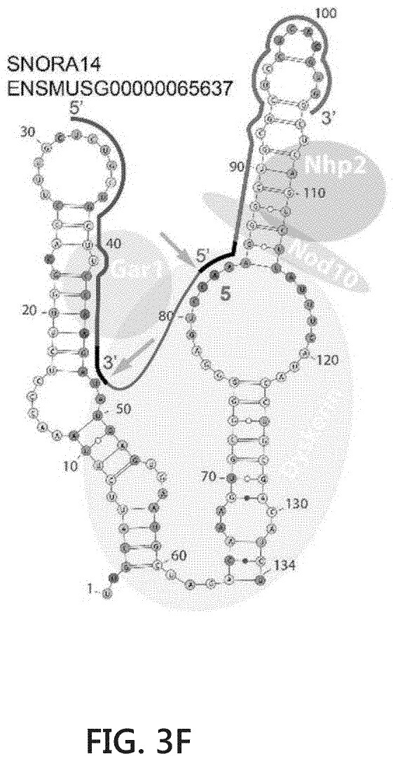

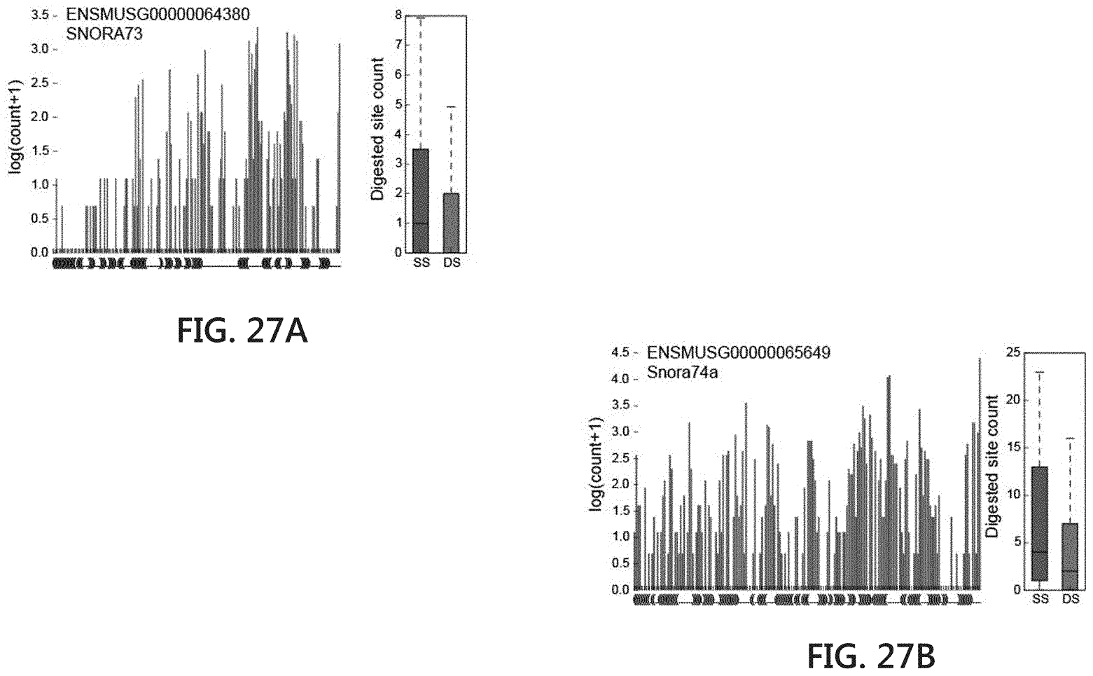

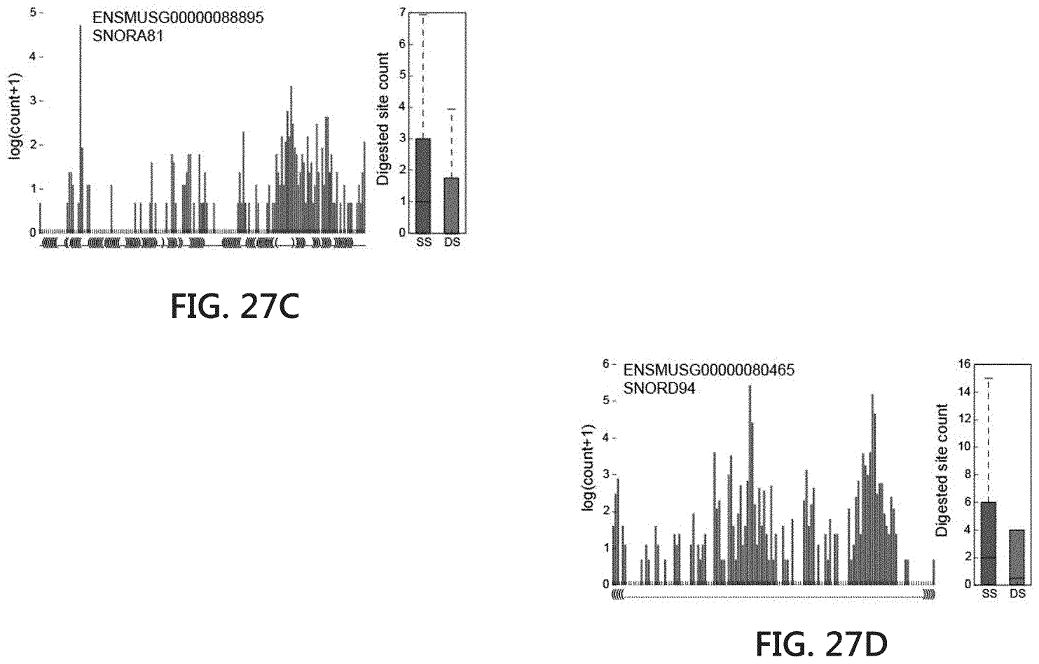

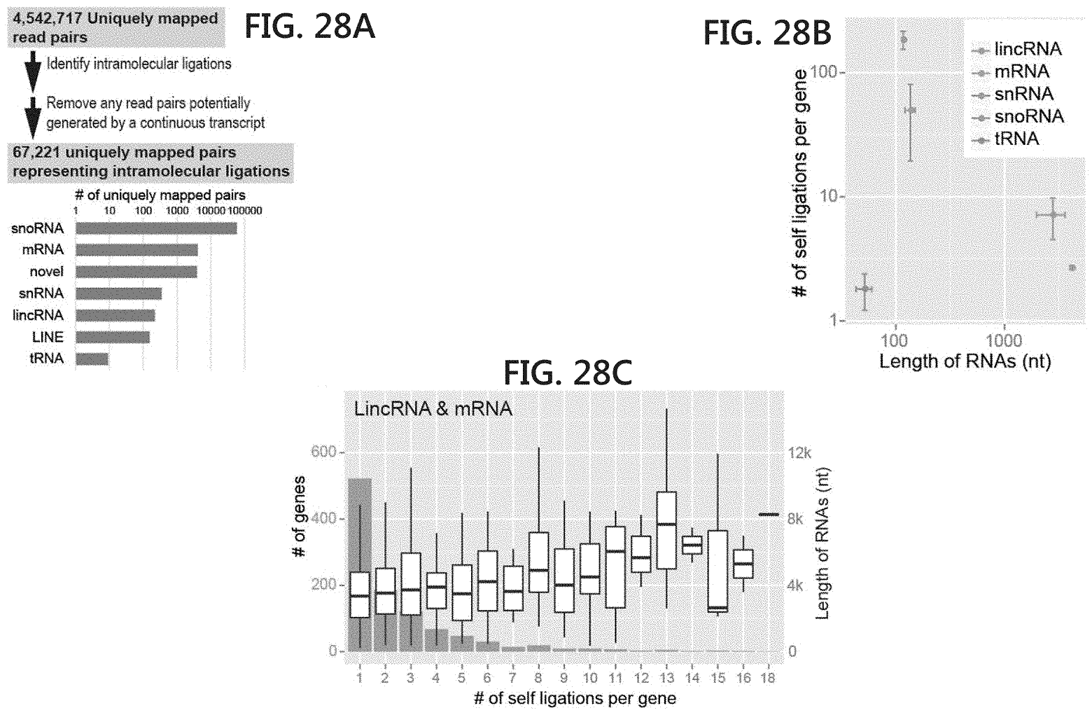

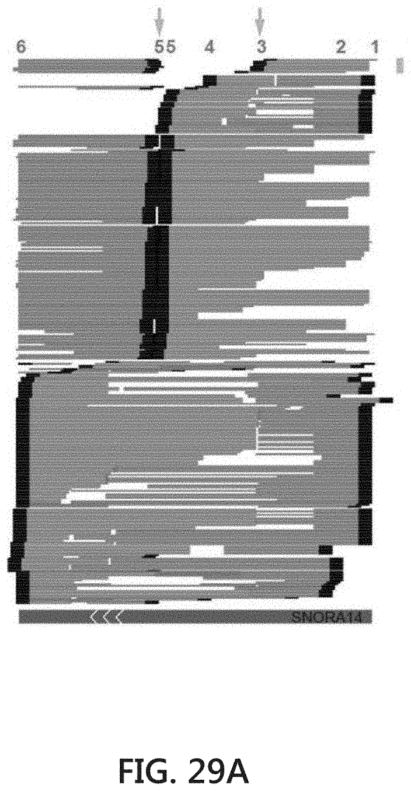

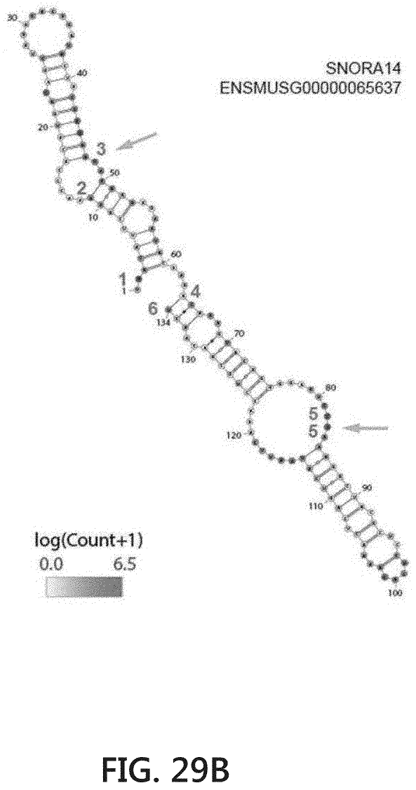

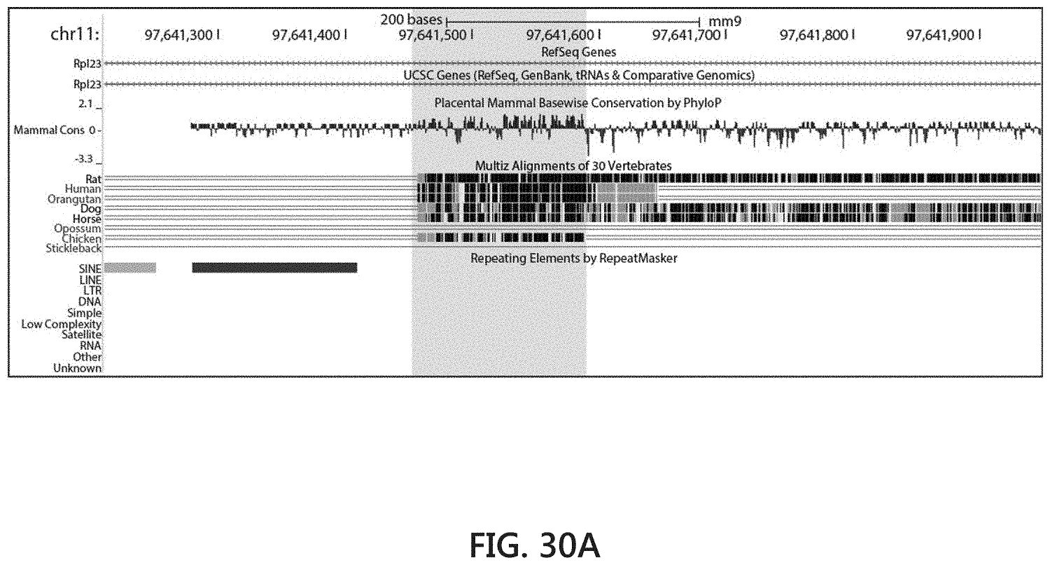

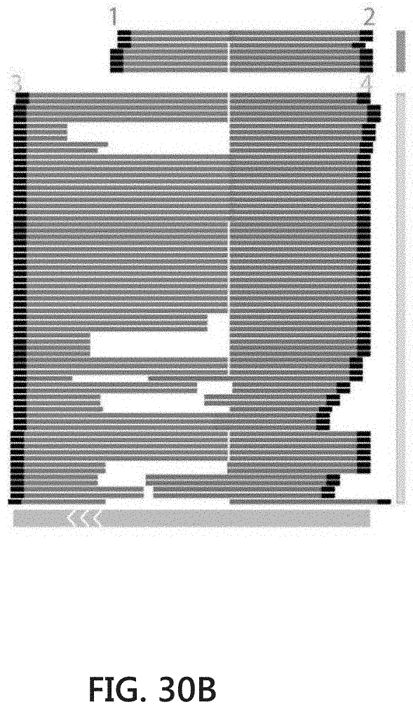

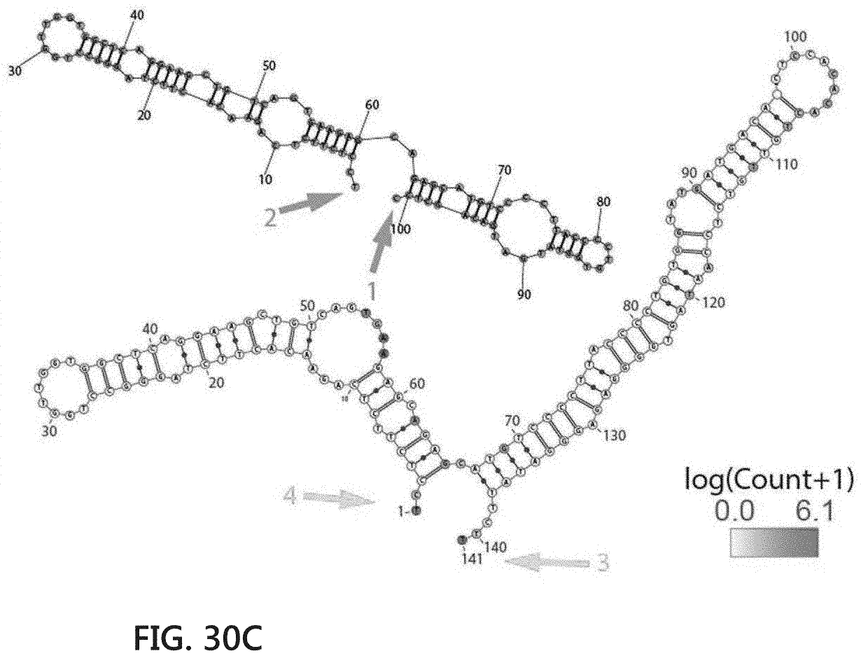

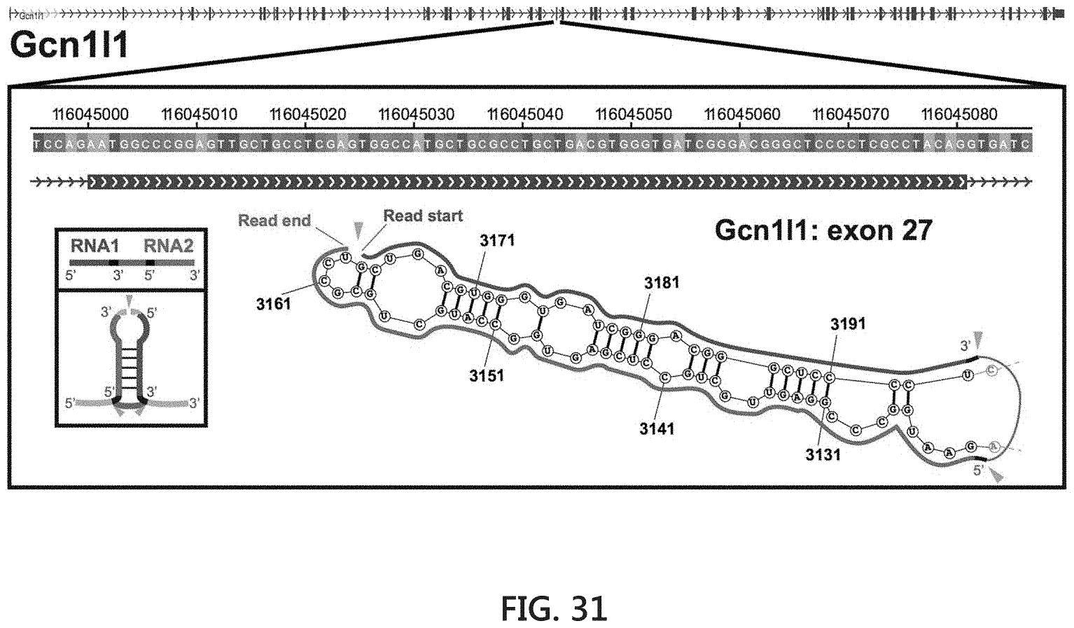

Although RNA Hi-C was originally designed for mapping inter-molecule interactions, it was found that RNA Hi-C revealed RNA secondary and tertiary structures. All the analyses above were based on inter-molecular reads. By looking at intra-molecular reads, several things can be learned about RNA structure. First, the footprint of single stranded regions of an RNA were identified by the density of RNase I digestion sites (RNase I digestion was applied before ligation, see Step 2 in FIG. 1A, FIGS. 27A to 27D). Second, the spatially proximal sites of each RNA were captured by proximity ligation (Step 5 in FIG. 1A). A total of 67,221 read pairs were mapped to individual genes, but were not within 2,000 bp of each other or on the same strand, and thus were generated from intra-molecule cutting and ligation (FIG. 28A). Each cut-and-ligated sequence can be unambiguously assigned to one of two structural classes by comparing the orientations of RNA1 and RNA2 in the sequencing read with their orientations in the genome (FIG. 3A). For example, 277 cut-and-ligated sequences were produced from Snora73 transcripts (FIG. 3B). The density of RNase I digestion sites (FIG. 3C) was strongly predictive of the single stranded regions of the RNA (heatmap, FIG. 3E). Six pairs of proximal sites were detected (circles, FIG. 3D). Each pair was supported by three or more cut-and-ligated sequences with overlapping ligation positions (black spots, FIG. 3B). Five out of the six proximal site pairs were physically close in the generally accepted secondary structure (arrows, FIG. 3E). On Snora14, a pair of inferred proximal sites appeared distant, according to sequenced inferred secondary structure (FIGS. 29A to 29B). However, ribonucleoprotein DYSKERIN bent Snora14 transcript in vivo (Kiss, T., Fayet-Lebaron, E. & Jady, B. E. Box H/ACA small ribonucleoproteins. Molecular cell 37, 597-606, doi:10.1016/j.molcel.2010.01.032 (2010)), making the two pseudouridylation loops close to each other, as predicted by the cut-and-ligated sequence (FIG. 3F). Structural information can even be derived on novel transcripts and some parts of mRNAs (FIGS. 30A to 30C and 31). To date, resolving the spatially proximal bases of any individual RNA remains a grand challenge. RNA Hi-C provides intra-molecule spatial proximity information for thousands of RNAs. Additionally, the single strand footprints of every RNA are mapped at the same time. Thus, RNA Hi-C largely expanded our capacity to examine RNA structures.

The key to mapping RNA interactions is selection. The introduction of a selectable linker in RNA Hi-C enabled an unbiased selection of interacting RNAs, making it possible to globally map an RNA interactome. The number of interacting partners per RNA in ES cells was strongly unbalanced, resulting in a scale-free RNA network. Interactions between long RNAs frequently used a small fraction of the transcripts. In analogy to protein interaction domains, the notion of RNA interaction sites was proposed. RNA interaction sites utilized base pairing to facilitate interactions of long RNAs, suggesting a new type of trans regulatory sequences. These trans regulatory sequences are more evolutionarily conserved than other parts of transcripts. RNA structure could be mapped by RNA Hi-C as well. Provided herein is an exemplary embodiment, where an RNA was bent by a protein, and such tertiary structure was revealed by the intro-molecule reads of RNA Hi-C. As such, this method and data should greatly facilitate future investigations of RNA functions and regulatory roles.

### Software Access

The RNA-HiC-tools software is available at http://systemsbio.ucsd.edu/RNA-Hi-C, the disclosure of which is incorporated herein by reference in its entirety.

### Materials and Methods

### Cell Culture

Undifferentiated mouse E14 ES cells were cultured under feeder-free conditions. ES cells were seeded on gelatin-coated dishes and were cultured in Dulbecco's modified Eagle medium (DMEM; GIBCO) supplemented with 15% fetal bovine serum (FBS; Gemini Gemcell), 0.055 mM 2-mercaptoethanol (Sigma), 2 mM Glutamax (GIBCO), 0.1 mM MEM nonessential amino acid (GIBCO), 5,000 U/ml penicillin/streptomycin (GIBCO) and 1,000 U/ml of LIF (Millipore). The cells were maintained in an incubator at 37° C. and 5% CO₂.

Mouse embryonic fibroblasts (MEFs) were cultivated in 15-cm dishes in DMEM (GIBCO) supplemented with 15% fetal bovine serum (FBS; Gemini Gemcell), 0.055 mM 2-mercaptoethanol (Sigma), 2 mM Glutamax (GIBCO), 0.1 mM MEM nonessential amino acid (GIBCO), 5,000 U/ml penicillin/streptomycin (GIBCO). MEFs were also maintained in an incubator at 37° C. and 5% CO₂.

Drosophila S2 cells (Invitrogen) were maintained in 15-cm plates in Schneider's Drosophila Medium (GIBCO) supplemented with 10% heat-inactivated fetal bovine serum (FBS; Gemini Gemcell), and 5 ml 1:100 Penicillin-Streptomycin (GIBCO) in an incubator at 28° C. without CO₂.

### Tissue Dissection and Preparation

Mice handling was approved by the Institutional Animal Care and Use Committee of the University of California San Diego. Adult female (C57BL/6J background) was sacrificed by cervical dislocation and the whole brain was immediately collected, rinsed with ice-cold PBS three times and snap frozen. Frozen whole mouse brain tissue was ground into fine powder in liquid nitrogen using a mortar and pestle. The tissue powder was quickly transferred into a Petri dish on a bed of dry ice and irradiated on dry ice three times at 400 mJ/cm2 in a UV cross-linker (254 nm) with gentle swirling between each irradiation. Cross-linked powdered tissue was immediately lysed and subjected to RNA Hi-C procedure as described.

### Overview of the RNA Hi-C Method

RNA Hi-C was designed to: (i) capture interacting RNAs in vivo in an unbiased manner without genetically or transiently introducing exogenous molecules; (ii) allow stringent removal of non-physiologic associations that form after cell lysis (S. Mili, J. A. Steitz, RNA 10, 1692 (2004)); (iii) select the proximity-ligated chimeric RNAs; (iv) allow unambiguous bioinformatic identification of interacting RNAs. These objectives can be achieved by: (i) cross-linking and immobilization of all RNA-protein complexes (a complex comprising protein and nucleic acid, intermediate proteins with nucleic acid or a protein complex bound to nucleic acid, wherein the nucleic acid is RNA) in streptavidin beads and removal of non-specific binding by denaturing conditions; (ii) attaching a biotin-tagged RNA linker to facilitate selective enrichment of chimeric RNA constructs; (iii) using the linker sequence to unambiguously split the interacting RNAs from a sequencing read pair.

### Step 1: Cross-Linking RNAs to Proteins

UV irradiation was used to form covalent bonds between photoreactive nucleotide bases and amino acids. UV irradiation generates highly reactive, short-lived states of the nucleotide bases within the RNA, inducing covalent bond formation only with amino acids at their contact points without additional elements that might cause conformational perturbation (I. G. Pashev, S. I. Dimitrov, D. Angelov, Trends in Biochemical Sciences 16, 323 (1991)). UV irradiation at 254 nm does not promote protein-protein cross-linking due to the different wave lengths absorbed by amino acids. Specifically, cells were washed twice in ice-cold PBS and irradiated with UV-C (254 nm) at 400 mJ/cm² in ice-cold PBS on ice. Cells were harvested by scraping and pelleted by centrifugation at 1,000×g for 5 min at 4° C. Cell pellets were snap-frozen in liquid nitrogen and stored at −80° C.

An RNA Hi-C library (ES-indirect) was generated in which protein-protein complexes were cross-linked as well. This was to capture the RNA that were brought together by protein interactions. An in vivo dual cross-linking method was applied with previously validated parameters (Illumina, “TruSeq® Samll RNA Sample Preparation Guide” (2014); P. Yu et al., Spatiotemporal clustering of the epigenome reveals rules of dynamic gene regulation. Genome research 23, 352 (February, 2013); N. J. Loman et al., Performance comparison of benchtop high-throughput sequencing platforms. Nature biotechnology 30, 434 (May, 2012)). Briefly, cells were first rinsed with room temperature PBS and treated with 1.5 mM EthylGlycol bis(SuccinimidylSuccinate) (EGS, Pierce Protein Research Products, Rockford, Ill.) freshly-prepared in PBS for 45 minutes at room temperature on a shaker. Cells were further treated with formaldehyde (Pierce Protein Research Products, Rockford, Ill.) to a final concentration of 1% and incubated for 20 minutes at room temperature with rocking. Glycine was added to a final concentration of 250 mM and incubated for 10 minutes at room temperature to quench the cross-linking reaction. Cells were then washed once with PBS at room temperature, scraped off, pelleted at 1,000×g for 5 min at 4° C., snap-frozen in liquid nitrogen and stored at −80° C.

A control experiment (ES-indirect) was conducted in which protein-protein complexes were cross-linked as well. This controls for the RNAs that were brought together by protein interactions. Thus, an in vivo dual cross-linking method was applied with previously validated parameters (S. K. Kurdistani, M. Grunstein, Methods 31, 90 (2003); D. E. Nowak, B. Tian, A. R. Brasier, BioTechniques 39, 715 (2005); J. Zhang et al., Methods 58, 289 (2012)). Briefly, cells were first rinsed with room temperature PBS and treated with 1.5 mM EthylGlycol bis(SuccinimidylSuccinate) (EGS, Pierce Protein Research Products, Rockford, Ill.) freshly-prepared in PBS for 45 minutes at room temperature on a shaker. Cells were further treated with formaldehyde (Pierce Protein Research Products, Rockford, Ill.) to a final concentration of 1% and incubated for 20 minutes at room temperature with rocking. Glycine was added to a final concentration of 250 mM and incubated for 10 minutes at room temperature to quench the cross-linking reaction. Cells were then washed once with PBS at room temperature, scraped off, pelleted at 1,000×g for 5 min at 4° C., snap-frozen in liquid nitrogen and stored at −80° C.

### Step 2: Cell Lysis, RNA Fragmentation, and Protein Biotinylation

Approximately 6×10⁸ cross-linked cells stored at −80° C. were thawed on ice and resuspended in ˜3 volumes of lysis buffer (50 mM Tris-HCl pH 7.5, 100 mM NaCl, 0.1% SDS, 1% IGEPAL CA-630, 0.5% sodium deoxycholate, 1 mM EDTA supplemented with 1:20 volume of EDTA-free complete protease inhibitor cocktail (Roche)). Lysis was performed on ice for 20 minutes. Cell debris and insoluble chromatin were removed by centrifugation at 20,000×g for 10 min at 4° C. The supernatant was collected and treated with TURBO DNase (Invitrogen) at concentration of 10 μl TURBO DNase per ml lysate for 20 minutes at 37° C. RNAs were digested into ˜1000-2000 nt (ES-1) or ˜1000 nt (ES-2) fragments by adding 10 μl of 1:100 diluted RNase I (NEB) per ml of lysate and incubating at 37° C. for 3 minutes. Following RNase I treatment, the lysate was immediately transferred to ice for at least 5 minutes. Both RNase I and sonication based fragmentation leave 5′-OH and 3′-P ends, incompatible with RNA ligation, which suppress undesirable RNA ligations. To stop DNase digestion, EDTA (Ambion) was added to a 25 mM final concentration and incubated the mixture at 4° C. for 15 minutes with rotation. The fragmented dual cross-linked (ES-indirect) lysate was prepared as follows: after the lysis on ice for 20 minutes the suspension was directly subjected to fragmentation by sonication (Covaris E220) under the following settings: 20 min with 5% duty cycle, 140 Watts peak incident power and 200 cycles per burst at 4° C.

For cross-species experiment (Fly-Mm), approximately 3×10⁸ E14 mES cells and 3×10⁸ Drosophila S2 cells were lysed separately and then mixed before protein biotinylation.

To dissociate loosely bound proteins, 500 mM NaCl final concentration was added and the solution was incubated at 4° C. for 10 minutes with rotation. To further dissociate protein complexes and non-cross-linked RNAs and halt the activities of RNase I, SDS was added to a 0.3% final concentration and incubated the mixture with shaking at 750 r.p.m. for 15 minutes at 65° C. After letting the solution mixture cool down to room temperature, the cysteine residues were biotinylated by adding to the lysate 1:5 volume of 25 mM (13.56 mg/ml) EZlink Iodoacetyl-PEG2-Biotin (IPB) (Pierce Protein Research Products) and rotating the mixture in the dark for 90 minutes at room temperature. The biotinylation reaction was quenched by adding DTT to a 5 mM concentration and incubating at room temperature for 15 minutes. To neutralize SDS, Triton X-100 (Sigma) was added to a 2% final concentration and incubated at 37° C. for 15 minutes. The lysate sample was dialyzed in a 20 kD cutoff Slide-A-Lyzer Dialysis Cassette (Pierce Protein Research Products, Rockford, Ill.) at room temperature in 2 litters of dialysis buffer (20 mM Tris-HCl pH 7.5, 1 mM EDTA) to remove excess biotin. The dialysis buffer was changed at least thrice, once every 2 hours. Following dialysis, the lysate was transferred to a 15 ml tube.

### Step 3: Immobilization on Beads

The protein-RNA complexes were immobilized at low bead-surface density on streptavidin-coated beads (800 μl MyOne Streptavidin T1 beads, which is equivalent to 200 cm² surface area). The advantages of immobilization on a solid surface include: (i) reduction of random intermolecular ligations between non-cross-linked oligonucleotides (R. Kalhor, H. Tjong, N. Jayathilaka, F. Alber, L. Chen, Nat Biotech 30, 90 (2012)), (ii) permit efficient buffer exchange, (iii) removal of non-physiologic interactions by stringent washes.

800 μl MyOne T1 beads were washed thrice with PBST (PBS with 0.1% Tween-20), resuspended in 800 μl of the same buffer and transferred into the biotinylated lysate. The bead-lysate suspension was rotated at room temperature for 45 minutes. During this incubation, 200 μl of neutralized 25 mM IPB was prepared by adding equal molarity of DTT and incubating at room temperature for at least 30 minutes. The beads were immobilized using a magnetic stand and most of the supernatant was aspirated out, leaving behind 4 ml of the supernatant. The beads were resuspended in the left-over solution followed by the addition of 200 μl of neutralized IPB. IPB was used to saturate excess of un-bound streptavidin after immobilization, which can interfere with subsequent step which involves biotin-tagged RNA linker. To remove the undesired RNAs non-covalently attached to proteins or via nonspecific protein-protein interactions (S. C. Kwon et al., Nat Struct Mol Biol 20, 1122 (2013); A. Castello et al., Nat. Protocols 8, 491 (2013)), the beads were washed three times with ice-cold denaturing washing buffer I (50 mM Tris-HCl pH 7.5, 0.5% lithium dodecyl sulfate, 500 mM lithium chloride, 7 mM EDTA, 3 mM EGTA, 5 mM DTT) with rotation at 4° C. for 5 minutes in every wash. Then the beads were washed with ice-cold high-salt wash buffer II (50 mM Tris-HCl pH 7.5, 1 M NaCl, 0.1% SDS, 1% IGEPAL CA-630, 1% sodium deoxycholate, 5 mM EDTA, 2.5 mM EGTA, 5 mM DTT), wash buffer III (1×PBS, 1% Triton X-100, 1 mM EDTA, 1 mM DTT), and PNK wash buffer (20 mM Tris-HCl pH 7.5, 10 mM MgCl₂, 0.2% Tween-20, 1 mM DTT); each buffer two times with rotation for 5 minutes at 4° C. during the second wash.

### Step 4: Ligation of a Biotin-Tagged RNA Linker

Next, a biotin-tagged RNA linker (5′-rCrUrArG/iBiodT/rArGrCrCrCr ArUrGrCrArArUrGrCrGrArGrGrA) (SEQ ID NO: 1) was attached to the RNA's 5′ end. The biotin-tagged linker serves as a selection marker to enrich for the ligated the RNAs; it also delineates a clear boundary to unambiguously split any sequencing read that covered a ligation junction. The 5′-end of the RNA linker was temporarily “blocked” from ligation to avoid linker circularization or concatenation. This was achieved by synthesizing the linker with a 5′-OH group, which is incompatible with ligation but can be “re-activated” by phosphorylation. However, RNase I leaves a 5′-OH end, which is incompatible for linker ligation, thus the 5′ end was first phosphorylated with T4 Polynucleotide Kinase (PNK), 3′ phosphatase minus (NEB). The wild-type T4 PNK was not used due to its additional 3′ phosphatase activities, which modifies the 3′-ends of RNAs from 3′-P into 3′-OH, making them susceptible to self-ligation.

This was achieved by removing wash buffer and subsequently resuspending the beads in 100 μl of PNK reaction mixture (73 μl of RNase-free water, 10 μl of 10×PNK buffer, 10 μl of 10 mM ATP, 5 μl of 10 U/μl T4 PNK (3′ phosphatase minus) (NEB), 2 μl of RNAsin Plus (Promega)) and incubating for 1 hour at 37° C. with intermittent shaking at 1,200 r.p.m. for 5 seconds every 2 minutes. The beads were washed with wash buffer I, II, III and PNK, each buffer two times with rotation for 5 minutes at 4° C. in the second wash. The ice-cold washes were used to eliminate any left-over PNK which can phosphorylate the RNA linker, inducing it to be potentially ligated to the 3′-end of RNAs. After wash buffer was remove, the biotin-tagged RNA linker was ligated to RNA 5′-ends by adding 160 μl RNA ligation reaction mixture which contained 2 μl RNAsin Plus (Promega), 16 μl of 10 mM ATP, 16 μl of 10×RNA ligase buffer, 16 μl of 1 mg/ml BSA, 30 μl of 20 μM biotin-labelled linker, 64 μl of 50% PEG8000 (NEB), 16 μl of 10 U/μl T4 RNA ligase 1 (NEB). Ligation was carried out at 37° C. for 1 hour and at 16° C. overnight with intermittent shaking at 1,200 r.p.m. for 15 seconds every 2 minutes. BSA was added to enhance the activities of T4 RNA ligase and prevent bead aggregation. PEG was used to enhance intermolecular ligation by increasing the concentrations of the donor and the acceptor ends (D. B. Munafó, G. B. Robb, RNA 16, 2537 (2010)).

### Step 5: Proximity Ligation

Next, the beads were washed twice with ice-cold wash buffer II, once with ice-cold wash buffer III, and PNK wash buffer. To prepare for proximity ligation, the RNA 3′-end was first dephosphorylated using the 3′ phosphatase activities of T4 PNK, leaving a 3′-hydroxyl group (I. Huppertz et al., Methods 65, 274 (2014)). After discarding wash buffer, the beads were mixed with 73 μl of RNase-free water, 20 μl of 5×PNK buffer pH 6.5 (350 mM Tris-HCl pH 6.5, 50 mM MgCl₂, 10 mM DTT), 5 μl of 10 U/μl T4 PNK (3′ phosphatase minus) (NEB), 2 μl of RNAsin Plus (Promega) and incubated for 20 minutes at 37° C. with intermittent shaking at 1,200 r.p.m. for 5 seconds every 2 minutes. The beads were washed once with PNK wash buffer and the 5′-end of the biotin-labelled linker was phosphorylated in 100 μl of PNK reaction mixture (73 μl of RNase-free water, 10 μl of 10×PNK buffer, 10 μl of 10 mM ATP, 5 μl of 10 U/μl T4 PNK (3′ phosphatase minus) (NEB), 2 μl of RNAsin Plus (Promega)) for 1 hour at 37° C. with intermittent shaking. Following phosphorylation, the beads were wash twice in PNK wash buffer and proximity ligation was then performed under extremely diluted conditions in a 15 ml total volume reaction (8.9 ml of RNase-free water, 1.5 ml of 10 mM ATP, 1.5 ml of 10×RNA ligase buffer, 75 μl of 20 mg/ml BSA (NEB), 25 μl of 1 M DTT, 2.25 ml of 100% DMSO, 0.75 ml of 10 U/μl T4 RNA ligase 1 (NEB)) to minimize inter-complex ligations. The proximity ligation was carried out at 37° C. for 1 hour and at 16° C. overnight with continuous rotation. Dimethylsulfoxide (DMSO) was added to a 15% (v/v) final concentration to stimulate ligation of highly structured RNAs.

### Step 6. Selection and Extraction of Desired RNA-RNA Interactions and Reverse Transcription

The following day, ligation was stopped by adding EDTA to a final concentration of 25 mM and rotating for 15 minutes at 4° C. to prevent inter-molecular ligation from happening as the beads were collected on the wall of the tube. The beads were washed once in PBST. The protein-RNA complexes were next eluted from streptavidin beads twice in 100 μl of Elution Buffer (100 mM Tris-HCl pH 7.5, 50 mM NaCl, 10 mM EDTA, 1% SDS, 10 mM DTT, 2.5 mM D-biotin (Invitrogen)) by heating to 95° C. for 5 minutes. The resulting solutions were combined, mixed with 50 μl of 800 U/ml Proteinase (NEB) and incubated at 55° C. for 2 hours. The mixture was then topped-up with RNase-free water to the final volume of 400 μl. RNAs were extracted in 400 μl of phenol:chloroform:isoamyl alcohol (125:24:1, pH 4.5) (Ambion) and incubation at 37° C. for 20 minutes with shaking at 1000 r.p.m. The mixture was transferred into a 2 ml MaXtract high density phase lock gel tube (Qiagen) and centrifuged at 16,000×g for 5 minutes at room temperature. Residual phenol was removed by adding 400 μl of chloroform to the same MaXtract tube and centrifugation at 16,000×g for 5 minutes at room temperature. Following centrifugation, the aqueous phase was transferred into a new tube and RNAs were precipitated by adding 1:9 volume of 3 M sodium acetate pH 5.2, 1.5 μl of glycoblue (Ambion) together with 1 ml of 1:1 ethanol:isopropanol and incubating at −20° C. overnight. The precipitated RNA was pelleted by centrifugation at 21,000 g for 30 minutes at 4° C. After discarding the supernatant, the pellet was washed twice with 80% ethanol and air-dried until ethanol completely evaporated. The purified RNAs at this stage were a mixture of RNAs without linkers (RNA1 or RNA2), RNAs ligated with linkers but not proximity-ligated with other RNAs (5′-linker-RNA2), and the desirable chimeric constructs in the form of 5′-RNA1-linker-RNA2. RNA1 can be depleted by selection of the biotin tagged linker. The non-informative 5′-linker-RNA2 was therefore depleted as well as in the next reaction with T7 exonuclease.

6.1.

Removing biotin from terminal linkers (5′-linker-RNA2). This was based on the RNase H activity of T7 exonuclease, which not only removes 5′ mononucleotides from duplex DNA but also exert exonucleolytic activity on the RNA strand from a RNA-DNA hybrid (K. Shinozaki, O. Tuneko, Nucleic Acids Research 5, 4245 (1978)). A complementary DNA oligonucleotide (5′-T*C*G*C*ATTGCATGGGCTACT AGCAT (SEQ ID NO: 2), where * denotes the phosphorothioate bond to block its digestion by T7 exonuclease (T. T. Nikiforov, R. B. Rendle, M. L. Kotewicz, Y. H. Rogers, Genome Research 3, 285 (1994)) was annealed to the RNA linker, creating a double stranded DNA-RNA hybrid between the RNA linker and the complementary DNA strand. The complementary DNA strand was designed so that after annealed, the 5′-end of the RNA linker was recessed while the 3′-end of the DNA strand was protruding. The annealed products were then treated with T7 exonuclease.

The RNA pellet was resuspended in 17 μl of RNase-free water, 4 μl of 10×NEBuffer4, 7 μl of 100 μM complementary DNA oligo. Annealing was performed by denaturing at 70° C. for 5 minutes and then slowly ramping down the temperature (at −0.1° C./s) to 60° C., incubating at 60° C. for another 5 minutes before slowly cooling down (−0.1° C./s) to 37° C. and incubating at 37° C. for 15 minutes. The annealed mixture was then mixed with 8 μl of 10 U/μl T7 exonuclease (NEB), 4 μl of 1 mg/ml BSA and incubated at 37° C. for 30 minutes and another 30 minutes at 30° C. The DNA oligonucleotides was removed as well as any contaminating genomic DNA using TURBO DNase rigorous treatment: 44 μl of RNase-free water, 10 μl of 10×TURBO DNase buffer, 6 μl of TURBO DNase (Invitrogen) was added and the resulting mixture was incubated at 37° C. for 1 hour. DNase-treated RNA was purified by phenol:chloroform extraction and ethanol precipitation as described above.

6.2.

Removal of rRNAs by antibody-based depletion of RNA-DNA hybrid (GeneRead rRNA Depletion Kit (Qiagen)) in ES-2, MEF samples. rRNA was removed according to the manufacturer's instructions with the following modifications. Instead of cleaning up depleted RNA by RNeasy MinElute spin columns which will remove RNAs shorter than 200 nucleotides, excess rRNA capture probes were removed by rigorous DNase-treatment. DNase-treated RNA was also purified by phenol:chloroform extraction and ethanol precipitation as described above.

6.3.

RNA shearing. Following ethanol precipitation, RNA was fragmented into size range of 150-400 bp, optimal for sequencing by Illumina HiSeq, by using the RNase III fragmentation kit according to the manufacturer's protocol. Fragmented RNA was purified by 2.2×SPRISelect beads (Beckman Coulter Genomics) and ethanol precipitated as described above.

6.4.

Ligation with reverse transcription adapter. Next, the RNAs were ligated with a 3′ reverse transcription (RT) adapter (/5rApp/AGATCGGAAGAGC GGTTCAG/3ddC/ (SEQ ID NO: 3)) that served as a primer for a RT reaction. Following ethanol precipitation, the RNA pellet was resuspended in 20 μl of ligation reaction mixture: 1 RNAsin Plus (Promega), 2 μl of 10×RNA ligase buffer, 7 μl of 20 μM pre-adenylated L3-App adapter, 8 μl of 50% PEG8000 (NEB), 2 μl of 200 U/μl T4 RNA ligase 2, truncated KQ (NEB). The reaction was incubated overnight at 16° C.

6.5.

Reverse transcription. Following ligation, RNA was purified by 2× SPRISelect beads (Beckman Coulter Genomics) and eluted in RNase-free water. The following RT reaction is described for 2 μg of RNA and was scaled up accordingly for higher amount of RNAs. For each experiment or replicate, a different RT primer containing individual experimental barcode sequence was used. Each RT primer has the form of 5′-/5Phos/NNXXXXNNNNAGATCGGAAGAGCGTCGTGgatcCTGAACCGCTCTTCCGAT CT (SEQ ID NO: 4). According to this scheme, the first read of every sequencing read pairs contains a barcode that takes the configuration of NNNNXXXXNN (SEQ ID NO: 5) (reverse complement of that from the RT primer), where the Ns are a random 6 nt barcode for removing PCR duplicates (G. B. Loeb et al., Molecular cell 48, 760 (Dec. 14, 2012); Z. Wang et al., PLoS Biol 8, e1000530 (2010); J. Konig et al., Nature structural & molecular biology 17, 909 (July, 2010); S. W. Chi, J. B. Zang, A. Mele, R. B. Darnell, Nature 460, 479 (Jul. 23, 2009)). Any two pair-end reads with identical mapped locations and random barcodes would be counted as only one. The XXXX is a fixed 4 nt sample barcode for multiplexed sequencing (AGGT for ES-1, CGCC for ES-2, CATT for ES-indirect, CGCC for MEF). Any two 4 nt sample barcodes differs by three nucleotides to avoid potential confusions from mutations or sequencing errors.

For cDNA synthesis, 9 μl of RNA was mixed with 1 μl 10 mM dNTPs and 1 μl of 50 μM RT primer. The mixture was heated at 65° C. for 5 minutes and snap-cooled in ice for at least 2 minutes. 4 μl of 5× First-Strand buffer (Invitrogen), 1 μl DTT 0.1 M, 1 μl RNasin Plus, 1 μl of 10 mg/ml T4 gene 32 protein (NEB) were added. The resulting mixture was incubated at 50° C. for 2 minutes before adding reverse transcriptase enzyme to minimize mispriming. Then 2 μl of 200 U/μl Superscript III reverse transcriptase (Invitrogen) was added to the solution. The RT reaction mixture was then incubated at 50° C. for 45 minutes, 55° C. for 20 minutes followed by 4° C. hold. Here, the heat-inactivation of reverse transcriptase enzyme was omitted in order to preserve the RNA-cDNA hybrids.

### Step 7. Biotin Pull-Down of Chimeric RNA-DNA Hybrids

Streptavidin-biotin affinity purification was used to enrich for chimeric RNA-DNA hybrids. This pull-down was carried out after the second RNA fragmentation and reverse transcription in order to allow a substantial fraction of the sequencing read pairs to cover the RNA-linker or linker-RNA junctions, in one end of the read pair.

Specifically, 50 μl of Myone C1 beads (Invitrogen) was prepared by washing twice with 1× Tween B&W buffer (5 mM Tris-HCl pH 8.0, 0.5 mM EDTA, 1 M NaCl, 0.05% Tween) and once with 1×B&W buffer (5 mM Tris-HCl pH 8.0, 0.5 mM EDTA, 1 M NaCl). The beads were then resuspended with 100 μl of 2)<B&W buffer (10 mM Tris-HCl pH 8.0, 1 mM EDTA, 2 M NaCl). The RT mixture was topped up with RNase-free water to the final volume of 100 μl before being combined with 100 μl C1 bead suspension and incubated at RT for 30 minutes with rotation. The beads were reclaimed and washed thrice with 1×B&W buffer before being transferred into a new tube, followed by washing once with TE buffer pH 8.0. Next, the cDNA strand was released from streptavidin beads by completely digesting the RNA strand in 50 μl RNase H elution mixture (39.5 μl of RNase-free water, 5 μl 10×RNase H reaction buffer, 0.5 μl 10% Tween-20, 5 μl 5 U/μl RNase H (NEB)) for 1 hour at 37° C. The beads were collected on the tube wall using a magnetic concentrator and the supernatant was collected in a new tube for subsequent manipulations. RNase H was inactivated by heating at 70° C. for 20 minutes. cDNA was purified by 2.2× SPRISelect beads (Beckman Coulter Genomics) (v/v).

### Step 8. Construction of Sequencing Library

Considering the UV-induced cross-link site sometimes stalls reverse transcription, resulting in truncated cDNAs that lack the 5′ adapter (Y. Sugimoto et al., Genome Biology 13, R67 (2012)), a circularization strategy was adopted that allowed for constructing sequencing libraries even from truncated cDNAs (I. Huppertz et al., Methods 65, 274 (2014)) (FIGS. 7A to 7B). The RT primer contained the adapter regions to prime PCR amplification by IIlumina PE PCR Forward Primer 1.0 (5′-AATGATACGGCGAC CACCGAGATCTACACTCTTTCCCTACACGACGCTCTTCCGATCT) (SEQ ID NO: 6) and PE PCR Reverse Primer 2.0 (5′-CAAGCAGAAGACGGCATACGAGATCGGTCT CGGCATTCCTGCTGAACCGCTCTTCCGATCT) (SEQ ID NO: 7), flanking a BamHI restriction site and a sequencing barcode.

8.1.

Circularization. cDNA was circularized by CircLigase II (Epicentre). Briefly, cDNA was eluted from SPRISelect beads in 20 μl CircLigase reaction mixture (12 μl of sterile water, 2 μl of CircLigase II 10× reaction buffer, 1 μl of 50 mM MnCl₂, 4 μl of 5M Betaine, 1 μl of 100 U/μl CircLigase II (Epicentre)) and incubated for 2 hours at 60° C. CircLigase II was inactivated by incubating the reaction at 80° C. for 10 minutes.

8.2.

Relinearization. A complementary DNA oligo was annealed to the RT primer, generating a short double-stranded region suitable for BamHI restriction. This strategy also prevents BamHI activities on other endogenous BamHI restriction sites. Next, BamHI were applied, creating linear cDNAs with adapters at both 5′ and 3′ ends to prime subsequent PCR amplification. Next, oligo annealing mixture (43 μl water, 6 μl 10× FastDigest Buffer (Fermentas), 5 μl 20 μM Cut_oligo (5′-GTTCAGGATCCACGACGC TCTTCAAAA/3InvdT/) (SEQ ID NO: 8) was added into the CircLigase II reaction. Annealing was carried out by heating to 95° C. for 2 minutes, followed by 71 cycles of 20 seconds each, starting from 95° C. and decreasing the temperature by 1° C. after every cycle down to 25° C. and holding at 25° C. 6 μl of FastDigest BamHI (Fermentas) was added and incubated at 37° C. for 30 minutes. Re-linearized cDNA was purified by 2×SPRISelect beads (Beckman Coulter Genomics) (v/v) and eluted in nuclease free water.

8.3.

First PCR pre-amplification and size selection. Single-stranded cDNA was first pre-amplified by PCR using a truncated version of PCR primers (forward primer DP5, 5′-CACGACGCTCTTCCGATCT (SEQ ID NO: 9); reverse primer DP3, 5′-CTGAACCGCTCTTCCGATCT) (SEQ ID NO: 10) with small number of cycles (6 cycles). It was found that the final libraries were less prone to be contaminated with undesirable smaller size fragments (primer-dimers, products which contain only the barcode and/or RNA linker) by doing size selection at this stage.

Six cycles of PCR were performed in a 40 μl reaction which contained 20 μl of NEBNext High-Fidelity 2×PCR Master Mix (NEB), 0.625 μM of each DP5/DP3 primer using the following temperatures: 1 cycle of initial denaturation at 98° C. for 30 seconds; 6 cycles of amplification with 98° C. for 10 seconds, 65° C. for 30 seconds, 72° C. for 30 seconds; followed by final extension at 72° C. for 5 minutes; and hold at 4° C. The PCR product was purified by 1.8×SPRISelect beads (v/v) and size-selected using E-gel EX 2% Agarose gels (Invitrogen). The DNA fragments between 150 bp and 350 were excised from the gel and purified using MinElute gel extraction kit (Qiagen).

8.4.

rRNA removal by duplex-specific nuclease (DSN) approach (H. Yi et al., Nucleic Acids Research 39, e140 (2011)) (ES-1, ES-indirect). To reduce rRNA cDNAs from ES-1 and ES-indirect library, ss-cDNA were also pre-amplified using the truncated PCR primer DP5/DP3. However, the PCR cycle number was increased until 80-100 ng of cDNA could be obtained after purification by 1.8×SPRISelect beads (Beckman Coulter Genomics) (v/v). The size selection by agarose gel was skipped as this would largely reduce the amount of DNA. The eluted DNA from SPRISelect beads was mixed with 4.5 μl hybridization buffer (2 M NaCl, 200 mM HEPES, pH 8.0) and sterile water (if necessary) to a final volume of 18 μl. The resulting mixture was denatured at 98° C. for 2 minutes and re-annealed at 68° C. for 5 hours on a thermal cycler. While the reaction mix tube was still in the thermal cycler, 20 μl of 68° C.-preheated 2×DSN buffer (Axxora) was added to the reaction mix, mixed well by pipetting up and down 10 times and incubated the reaction for 10 minutes at 68° C. 2 μl of 1 U/μl DSN enzyme (Axxora) was added, mixed, and incubated at 68° C. for 25 more minutes. The reaction was stopped by adding 40 μl of 2×DSN stop solution (Axxora) to the reaction mix tube, mixing well and transferred the tube to ice. The reaction mixture was then purified using 1.8×SPRISelect beads.

8.5.

Final PCR amplification. PCR amplification was performed on the DNA produced from previous steps using full-length PCR primer PE 1.0 and 2.0 (Illumina). The number of PCR cycles was carefully titrated by running pilots PCRs with small aliquots of DNA to avoid over-amplification. The PCR products were purified by 1.8×SPRISelect beads (v/v) and size-selected fragments between 250-550 (120-420 bp insert plus ˜130 bp, the combined length of Illumina PE 1.0/2.0). Final libraries were quantified by Qubit (Invitrogen) and qPCR, quality-checked by Bioanalyzer (Agilent Technologies) and submitted for paired-end sequencing on Illumina HiSeq platform.

### Oligonucleotide Sequences Used in RNA Hi-C

The custom-designed RNA and DNA oligonucleotides used in the procedure are:

Biotinylated RNA linker (RNase-free HPLC-purified from IDT):

(SEQ ID NO: 11)
5′-rCrUrA rG/iBiodT/rA rGrCrC rCrArU rGrCrA rArUrG
rCrGrA rGrGrA-3′

Complementary DNA strand with RNA linker (RNase-free HPLC-purified from Sigma):

(SEQ ID NO: 12)
5′-T*C*G*C*ATTGCATGGGCTACTAGCAT-3′

Pre-adenylated RT adapter (RNase-free HPLC-purified from IDT):

(SEQ ID NO: 13)
5′-/5rApp/AGATCGGAAGAGCGGTTCAG/3ddC/

RT primers (adapted from (I. Huppertz et al., Methods 65, 274 (2014))) (RNase-free HPLC-purified from Sigma):

RT Primer for the ES-1 sample:

(SEQ ID NO: 14)
5′-/5Phos/NNAGGTNNNAGATCGGAAGAGCGTCGTGgatcCTGAACCG
CTCTTCCGATCT

RT Primer for the ES-2 and MEF samples (sequenced on different lanes):

(SEQ ID NO: 15)
5′-/5Phos/NNCGCCNNNNAGATCGGAAGAGCGTCGTGgatcCTGAACC
GCTCTTCCGATCT

RT Primer for the ES-indirect sample:

(SEQ ID NO: 16)
5′-/5Phos/NNCATTNNNNAGATCGGAAGAGCGTCGTGgatcCTGAACC
GCTCTTCCGATCT

Cut_oligo (HPLC-purified from IDT)

(SEQ ID NO: 17)
5′-GTTCAGGATCCACGACGCTCTTCAAAA/3InvdT/-3′

BamHI restriction site is underlined and in bold print.

Truncated PCR Forward Primer DP5 (HPLC-purified from IDT):

(SEQ ID NO: 18)
5′-CACGACGCTCTTCCGATCT

Truncated PCR Reverse Primer DP3 (HPLC-purified from IDT):

(SEQ ID NO: 19)
5′-CTGAACCGCTCTTCCGATCT

Illumina PE PCR Forward Primer 1.0 (PAGE-purified from Sigma):

(SEQ ID NO: 20)
5′-AATGATACGGCGACCACCGAGATCTACACTCTTTCCCTACACGACGC
TCTTCCGATCT

Illumina PE PCR Reverse Primer 2.0 (PAGE-purified from Sigma):

(SEQ ID NO: 21)
5′-CAAGCAGAAGACGGCATACGAGATCGGTCTCGGCATTCCTGCTGAAC
CGCTCTTCCGATCT

### The Computational Pipeline (RNA-HiC-Tools)

RNA-HiC-tools is a package of command-line tools for analyses of RNA Hi-C data. It is written in Python and R and is version controlled by GitHub. The full documentation is at http://systemsbio.ucsd.edu/RNA-Hi-C. The pipeline takes pair-end sequencing reads as input (FIG. 15A). The oligonucleotide sequences of the RNA linker and the sample barcodes used for multiplexed sequencing should also be provided to the pipeline. The main outputs include: 1. a parsed cDNA library, including the list of chimeric cDNAs in the form of RNA1-Linker-RNA2 (see the final product in FIGS. 7A to 7B, 15C), 2. the genomic locations of RNA1 and RNA2 of every chimeric cDNA (FIG. 15D), 3. interacting RNA pairs inferred from statistical enrichment of chimeric cDNAs (FIG. 15E). The analysis steps are as follows.

### 1. Removing PCR Duplicates

The forward read (Read1 in FIG. 15A) contains a 4 nt sample barcode and a 6 nt random barcode at the 5′ end. A read pair was classified as a PCR duplicate of another read pair and is therefore discarded if the two read pairs had identical sequences and contained identical barcodes (10 nt). The tool ‘remove_dup_PE.py’ provides this function, and generates a fastq/fasta file containing the non-duplicated reads, and reports the number of duplicates removed.

2. Assigning Multiplexed Sequencing Reads into Corresponding Experimental Samples

The tool ‘split_library_pairend.py’ assigns each pair-end read into a sample by matching the sample barcode in each read with those in the list of sample barcodes (a user input text file), generates a fastq/fasta file for the reads assigned to each sample, as well as a fastq/fasta file for the unassigned reads.

3. Recovering the cDNAs in the Sequencing Library

This step identifies the overlapping regions of the two ends of every read pair, if any. It also recovers the entire sequences of the cDNAs in the sequencing library, whenever possible.

If an overlap existed, this read pair was sequenced from a cDNA between 100 bp and 200 bp (not counting the lengths of P5 and P7) (Type 2, FIG. 32). In this case the entire sequence of the cDNA was completely covered by concatenating the forward read (Read 1) with the non-overlapping region of the reverse read (Read2).

If the cDNA was shorter than 100 bp, the presence of the P5 and the P7 primers at the two ends of the cDNA were verified (Type 1). The ones did not contain P5 or P7 were discarded (Type 4).

Without an overlap, the read pair was sequenced from a cDNA longer than 200 bp, whose sequence can only be partially recovered (Type 3, FIG. 32).

This function is achieved by ‘recoverFragment.py’, which uses local alignment to identify the overlapping regions. When the overlap was small (15 bp or less) compared to read length (100 bp on each end), local alignment could be insensitive. To overcome this insensitivity, ‘recoverFragment.py’ collects the read pairs without identifiable overlaps after the first alignment (ALIGN1, FIG. 32), truncates each read into one third of its length (retaining 33 bp at the 3′ of each read), and repeats local alignment (ALIGN4).

4. Parsing the Chimeric cDNAs

This step categorizes the cDNAs based on their configurations (FIG. 15C). This takes the completely (Type 1 and Type 2, FIG. 32) and partially recovered (Type 3) cDNA sequences, as well as the linker sequence as inputs. It identifies the location of the linker in the cDNA, and generates five categories of cDNAs based the locations of the linker sequence, including:
1. No linker. Any Type 1 or Type 2 cDNA that does not contain the linker sequence belongs to this category. This category can be further classified into three subsets, including:
   a. Barcode only. The entire cDNA was the 10 nt barcode (4 nt sample barcode+6 nt random barcode), most likely results of contamination of the unligated RT primers.
   b. Single RNA. The entire cDNA was a continuous fraction of an RNA.
   c. RNA1-RNA2. These were likely produced from a proximity ligation prior to the linker ligation.

Four linker-containing categories, including:
2. RNA1-Linker-RNA2. These were generated from the desirable chimeric RNAs. Any linker-free Type 3 cDNA, whose two reads were completed aligned two distinct RNA genes, was put into this category as well. It was required that both RNA1 and RNA2 sides contained at least 5 bp sequences.
3. Linker-RNA2. A linker was successfully ligated to the 5′ end of an RNA, but it was not succeeded by a proximity ligation.
4. RNA1-Linker. A linker was ligated to the 3′ end of an RNA. This was likely generated from RNAs or RNA fragments with a 3′-OH group, or cutting off the other RNA (RNA2) from the RNA1-Linker-RNA2 chimeras during the 2nd fragmentation step.
5. LinkerOnly. The entire cDNA was a barcode and a linker sequence.
   This step outputs the list of cDNAs belonged to the RNA1-Linker-RNA2 category.

### 5. Mapping to the Genome

Hereafter, all analyses were based on the RNA1-Linker-RNA2 type of read pairs. First, any cDNA containing less than 15 bp on either the RNA1 or RNA2 side of linker was discarded, because it is unlikely to uniquely map a 15 bp or less sequence to the genome in the mapping step. Then the two RNA fragments on each side of the linker (RNA1 and RNA2) were separately mapped to the mouse genome mm9/NCBI37 using Bowtie version 0.12.7 (B. Langmead, C. Trapnell, M. Pop, S. L. Salzberg, Genome Biology 10, (2009)), and parameters -f -n 1 -l 15 -e 200 -p 9 -S. This step, implemented in ‘Stitch-seq_Aligner.py’ outputs the read pairs where both RNA1 and RNA2 were uniquely mapped to the genome.

A potentially more sensitive mapping method was tested using Bowtie2 (B. Langmead, S. L. Salzberg, Nat Methods 9, 357 (April, 2012))'s “—sensitive-local” mode, with parameters “-D 15 -R 2 -N 0 -L 20 -i S,1,0.75”. This “multiseed alignment” used 20 bp seeds, allowing for 0 mismatches in any seed, 9 bp intervals (ceil (1+0.75×√100) between seeds, up to 15 consecutive seed extension attempts, and up to 2 times of “re-seeding”. It turned out that this alternative strategy identified slightly fewer unique alignments than Bowtie 0.12.7. The Bowtie 0.12.7 results were therefore passed into the next steps.

### 6. Identifying Interacting RNA Pairs

The annotations were retrieved from Ensembl (release 67, mouse NCBIM37), including the genes of mRNAs, lincRNAs, rRNAs, snRNAs, snoRNAs, miRNAs, misc_RNAs, tRNAs, and transposons. The different genomic copies of the same transposon were considered as different genes in this analysis. The reads mapped to rRNAs were removed from further analysis. The number of uniquely aligned reads (from either RNA1 or RNA2 of the RNA1-Linker-RNA2 type) were counted on every gene. Any gene with a read count less than 5 was filtered out. Next, the association between any two genes was tested with Fisher's exact test. The null hypothesis was that gene A and gene B independently contributed to the sequencing reads. The alternative hypothesis was that their contributions to read counts were associated. c_{A}, c_{B} were denoted as the read counts for gene A and gene B, respectively, and I_{A, B} as the read counts of co-appearance, where the two genes co-appeared on the same read pair. A Fisher's exact test was carried out on each gene pair, with I_{A,B}, c_{A}, c_{B}, c_{A}, c_{B}, c̅_̅{̅A̅}̅, c̅_̅{̅B̅}̅ as the test statistics, where c̅_̅{̅A̅}̅(c̅_̅{̅B̅}̅) was the read counts on other genes besides gene A (gene B). Both p-values and FDRs (Benjamini-Hochberg procedure (Y. Benjamini, Y. Hochberg, Journal of the Royal Statistical Society. 57, 289 (1995)) were calculated for every gene pair. This step outputs gene pairs with FDR<0.05 and fold-change (FC)≥3. The FC was calculated as (I′_{A, B}+0.5)/(I′_{A, B}+0.5), where I_{A,B}, was the co-appearing read counts in the control sample (ES-indirect). This step was implemented in ‘Select_strongInteraction_RNA.py’ which outputs strong interacting RNA pairs with information of their interaction regions, number of supporting pairs, p-value of significance, FDR and fold changes.

### 7. Identifying RNA Interaction Sites

The RNA interaction site was defined as a continuous RNA segment that frequently contributed to RNA-RNA interactions. RNA interaction sites were inferred from RNA Hi-C data as continuous RNA segments with multiple overlapping reads and frequent co-appearance (proximity ligation) with other RNAs. First, any continuous RNA segment covered by 5 or more uniquely aligned reads was identified as a candidate interaction site. Second, the association between any two candidate sites were tested with Fisher's exact test. The null hypothesis was that candidate sites A and gene B independently contributed to the sequencing reads. The alternative hypothesis was that their contributions to read counts were associated. c_{A}, c_{B}, was denoted as the read counts for candidate sites A and B, respectively, and I_{A,B} as the read counts of co-appearance, where the two sites co-appeared on the same read pair. A Fisher's exact test was carried out on each site pair, with I_{A,B}, c_{A}, c_{B}, c_{A}, c_{B}, c̅_̅{̅A̅}̅, c̅_̅{̅B̅}̅ as the test statistics, where c̅_̅{̅A̅}̅ (c̅_̅{̅B̅}̅) was the read counts on other candidate sites besides A (B). Both p-values and FDRs (Benjamini-Hochberg procedure) were calculated for every pair of candidate sites. The candidate sites exhibiting significant associations (FDR<0.05) were regarded as RNA interaction sites. This step was automated in ‘Select_strongInteraction_pp.py’ which outputs the identified RNA interaction sites.

The tool ‘Plot_interaction.py’ was developed for visualizing RNA interaction sites and the ligation events of these sites (FIGS. 16A-16B). Given any two genomic regions as input, for example the locations of two genes, this tool displays all the supporting read pairs in the form of RNA1-Linker-RNA2, where RNA1 and RNA2 were aligned to each of the two genomic locations. The linker of each RNA pair was plotted as well. This tool also plots RNA interaction sites in the input regions, if any, as well as the identified interactions between these sites.

The tool ‘Plot_Circos.R’ provides a global view of the RNA-RNA interactome (FIG. 16C). It plots the entire genome as a circle, and any RNA-RNA interaction as a curved line connecting two contributing genes. The interactions involving different types of RNAs are coded with different colors. The densities of RNA1 and RNA2 read fragments are displayed along with every chromosome as inner circles. Other analysis and visualization tools are described in http://systemsbio.ucsd.edu/RNA-Hi-C.

### Binding Energies Between RNA Interaction Sites

The binding energies between two RNA interaction sites were calculated by the DuplexFold program from RNAstructure version 5.6 (S. Bellaousov, J. S. Reuter, M. G. Seetin, D. H. Mathews, Nucleic Acids Res 41, W471 (July, 2013)). The base paring between two interaction sites was determined by MiRanda version 3.3a (D. Betel, A. Koppal, P. Agius, C. Sander, C. Leslie, Genome Biol 11, (2010).

### Conservation Levels of RNA Interaction Sites

For every read pair in the RNA1-Linker-RNA2 category (output of Step 4), the PhyloP conservation scores were obtained (G. M. Cooper et al., Genome Res 15, 901 (July, 2005)) of two 1000 bp genomic regions, one centered at the ligation junction of RNA1-Linker and the other centered at the ligation junction of Linker-RNA2. The average PhyloP scores of all the RNA1-Linker-RNA2 type read pairs were plotted. As a control, average PhyloP scores from the same number of random genomic regions of the same lengths were obtained.

### Network Analysis

The identified RNA-RNA interactions (output of Step 6) were converted to tabular format and imported into Cytoscape 3.1.0 (R. Saito et al., Nat Methods 9, 1069 (November, 2012)) for visualization. Each node represents a gene and is color-coded by the gene type. The degree of each node was calculated by Cytoscape.

Detecting Read Pairs Generated from Intra-Molecule Cutting and Ligation

Starting from the RNA1-Linker-RNA2 type of read pairs (output of Step 6), the following filters to identify the pair-end reads generated from self-interacting RNAs were applied:
1. Read pairs that mapped to two different genes were removed.
2. If a read pair mapped to the same gene, pairs were also removed that: (1) did not contain any fraction of the linker sequence; (2) the forward and the reverse reads mapped to opposite strands within 2000 bp; (3) the read mapped to plus strand has smaller coordinates than the read mapped to minus strand in the genome within the pair. This step minimizes the inclusion of any intact (continuous) RNA fragment in the structural analysis.

### RNA Folding and Secondary Structure Prediction

Structural information of the RNAs with known or generally accepted structures was downloaded from fRNAdb database v3.4 (T. Mituyama et al., Nucleic Acids Research 37, D89 (January, 2009)) in DOT format (graph description language). Figures were drawn from the DOT files using the command line version of VARNA Applet version 3.9 (K. Darty, A. Denise, Y. Ponty, Bioinformatics 25, 1974 (Aug. 1, 2009)). For the RNAs without structural information in fRNAdb, their secondary structures were predicted based on the sequence using the “Fold” program in RNAstructure version 5.6 (S. Bellaousov, J. S. Reuter, M. G. Seetin, D. H. Mathews, Nucleic Acids Res 41, W471 (July, 2013)).

### Control Experiments for RNA Hi-C

The first control experiment skipped the cross-linking step in the procedure. The second control experiment skipped the protein biotinylation step. The third control experiment carried out the entire procedure on the mixed cell lysate of mouse ES cells and Drosophila S2 cells.

A non-cross-linking control with approximately 3×10⁸ mouse ES cells was first carried out. The RNAs immobilized with proteins on streptavidin beads were purified by protein digestion as previously described. The purified RNAs were subjected to quantification by Qubit RNA HS assay (Invitrogen). The RNAs were below the detection limit of the assay (250 pg/μl). The sample volume was 20 μl (the same as previously described), which suggests that the RNA abundance was no more than 5 ng. At this point, the experiment was stopped because there was no chance to accomplish linker selection and library construction. In previously described experiments, the purified RNAs would be in the μg range at this step.

Second, another control was performed by not doing protein biotinylation (keeping cross-linking) with 3×10⁸ mouse ES cells. It turned out the RNAs purified from the beads were below the detection limit of Qubit RNA HS assay.

Third, the experiment was started with 3×10⁸ Drosophila S2 cells and 3×10⁸ mouse ES cells (cross-species control). The cells were cross-linked and lysed. The lysate from the two cell lines were mixed before protein biotinylation and proximity ligation. The mixture was subjected to the rest of the experimental procedure to produce a sequencing library (Fly-Mm). Fly-Mm contained 27,748,688 read pairs. After removing duplicate reads and splitting by the linker, there were 16,881,326 RNA1-RNA2 pairs. Each RNA part (either RNA1 or RNA2) was mapped to the fly genome (dm6) and mapped to the mouse genome (mm9). A total of 7,188,769 pairs had at least one part (either RNA1 or RNA2) that was not mappable to either mouse or fly genome. The rest 9,692,557 RNA1-RNA2 pairs had both parts mapped to the genomes, among which 8,484,807 pairs had each RNA part uniquely mapped to only one genome. The distribution of these mapped RNA pairs is as follows (Table 6). The proportion of RNA pairs mapped to two species is 0.52% (44,229/8,484,807).

Furthermore, it was inquired what would happen if the ES-1 library (pure mouse sample) were to be subjected to the analysis above. It turned out that 0.55% of the RNA1-RNA2 pairs would have one RNA part mapped uniquely to the mouse genome and the other part mapped uniquely to the fly genome. Therefore, the “contamination rate” for Fly-Mm sample (0.52%) was even smaller than that of the ES-1 sample (0.55%), suggesting that the experimental contamination (supposedly due to random ligation) was so low that it fell into the error range of the informatics procedure.

### Differences Between Dual Cross-Linking and UV Cross-Linking

FA-DSG dual cross-linking was compared to psoralen cross-linking and formaldehyde (FA) cross-linking in RAP-sequencing (J. M. Engreitz et al., RNA-RNA interactions enable specific targeting of noncoding RNAs to nascent Pre-mRNAs and chromatin sites. Cell 159, 188 (Sep. 25, 2014)). After cross-linking, Engreitz et al. used antisense oligonucleotides to purify nuclear Malat1 RNA, and sequenced the RNA that were purified together with Malat1. Engreitz et al. found little overlap of the Malat1 targets between dual cross-linking and the other two cross-linking methods. Except for one RNA, the hundreds of RNAs co-purified with Malat1 in the dual cross-linking were all unique (Supplementary Table 3 of Engreitz et al.). Engreitz et al. attributed this to the idea that dual cross-linking could “efficiently capture RNAs linked indirectly through multiple protein intermediates.” UV cross-linking (our method) was less effective than psoralen in nucleic acid to nucleic acid cross-linking, and was less effective than FA overall. Based on the published data, it was not expected that the detected RNA pairs by UV cross-linking and dual cross-linking strongly overlap.

More specifically, snoRNAs are short (˜150 nt) and are likely wrapped around or within the snoRNP protein complex when interacting with mRNA. Dual cross-linking is expected to retain the entire snoRNP complex. The snoRNP complex is expected to hinder RNase I from cutting snoRNA and also hinder RNA ligation. Therefore, large differences in the detected interactions involving snoRNA was expected.

Other RNAs with miRNA-Like Interactions.

It was inquired whether other RNAs could experience a similar process to miRNA biogenesis and also interact with mRNAs. The RNA Hi-C identified interacting RNAs with those found by small RNA sequencing (smallRNA-seq) and those bond to the AGO protein (HITS-CLIP) in ES cells. The smallRNA-seq selectively sequenced, “miRNAs and other small RNAs that have a 3′ hydroxyl group resulting from enzymatic cleavage by Dicer or other RNA processing enzymes”. Besides miRNA, other RNA types including snoRNA, pseudogeneRNA, mRNA UTRs also contributed to the small RNA pool, and were attached to AGO (FIGS. 17A to 17D). Moreover, large portions of RNA Hi-C identified interacting RNA pairs co-appeared in AGO HITS-CLIP data (FIG. 18). This data suggest there are non-miRNAs that are digested by DICER or other RNA processing enzymes and are incorporated into the RISC complex.

To elucidate what types of non-miRNA genes were most likely to undergo miRNA-like biogenesis, the RNA Hi-C identified RNA-RNA interactions to the following filters were subjected:

1. the interaction involves one mRNA (dubbed target) and one other RNA (source RNA);
2. the source RNA is processed into small RNA by enzymatic cleavage (FPKM>0 in smallRNA-seq);
3. both the target and the source RNAs appear in AGO HITS-CLIP (FPKM>0 for both RNAs);
4. the RNA Hi-C identified interaction sites on the source and the target RNAs exhibit strong base pairing (p-value <0.05, Wilcoxon signed-rank test comparing the binding energies between the RNA1 and RNA2 sequences of every pair-end read to the binding energies of randomly shuffled nucleotide sequences).

A total of 302 RNA-RNA interactions passed these filters. The majority (79%) of the source RNAs in these interactions were snoRNAs (Table ST2). The snoRNAs were prioritized for functional analysis.

It was hypothesized that a large number of snoRNAs were enzymatically processed into miRNA-like short RNAs and interact with mRNAs. This hypothesis was supported by 919 RNA Hi-C identified snoRNA-mRNA interactions where both the mRNA and the snoRNA were bound by AGO. Furthermore, AGO bound snoRNAs and their interacting mRNAs exhibited anti-correlated expression changes during guided differentiation of ES cells toward mesendoderm (P. Yu et al., Spatiotemporal clustering of the epigenome reveals rules of dynamic gene regulation. Genome research 23, 352 (February, 2013))(FIG. 17B). Additionally, AGO bound snoRNAs and their target mRNAs exhibited stronger base pairing than that without AGO binding (FIG. 17C). Finally, the small RNAs processed from snoRNAs referentially interacted with the UTR regions of mRNAs. Out of the 497 snoRNAs involved in RNA-RNA interactions, 243 interacted with UTR regions, among which 223 (92%) were detected in smallRNA-seq, suggesting the experience of an enzymatic cut (FIG. 17D). In comparison, the other 254 snoRNAs interacting with non-UTR regions contained fewer (55%) small RNAs. Besides, two times more UTR-interacting sno-siRNAs were AGO bound than the non-UTR interacting snoRNAs (p-value <2.2⁻¹⁶, Chi-square test). For example, Snora14 RNA targeted the 3′ UTR of Mcl1 mRNA (FIG. 19A). The interacting site on Snora14 RNA (110-135 nt) precisely overlapped with the enzymatically processed small RNA (light purple lane) as well as the AGO bound region (green lane). The enzymatically processed portion of Snora14 RNA is located completely on one side of a hairpin loop (FIG. 19B), and exhibits a strong binding affinity (−60 kCal/mol) to the target site on Mcl1 UTR. The expression of the processed Snora14 RNA was negatively correlated with that of Mcl1 mRNA (FIG. 19C). Taken together, this data suggest a large number of small interfering RNAs originated from snoRNA genes, which interact with more than 900 mRNAs in ES cells.

Mapping RNA-RNA Interactome and RNA Structures In Vivo without Perturbation

It remains formidable to analyze the entire RNA-RNA interactome. The RNA Hi-C technology was developed to map RNA-RNA interactions embraced by any single protein in vivo, without any perturbation. The RNA-RNA interactome was systematically mapped in embryonic stem cells, revealing 46,780 interactions. 7 interactions were validated using RAP-seq 1. In this interactome the majority of miRNAs and lincRNAs each specifically interacted with one mRNA, which contradicts the current dogma of “promiscuous” RNA interactions. Base pairing was observed at the interacting regions between long RNAs, suggesting a class of regulatory sequences acting in trans. In addition, RNA Hi-C provided new information on RNA structures, by simultaneously revealing the footprint of single stranded regions and the spatially proximal sites of each RNA. This technology vastly expands the identifiable portion of an RNA-RNA interactome, without perturbing the endogenous level of RNA expression.

Simulation analysis of RNA Hi-C.

Data Synthesis.

In order to estimate the sensitivity and specificity of RNA Hi-C, including its experimental and computational procedures, a simulation analysis was carried out. 1,000,000 pair-end reads was simulated by computationally mimicking the data generation process. The parameters used for the simulation were derived from real data. The simulated data generation process is as follows.

For each pair-end read (2×100 bases):

1. Choose a sample barcode from the four sample barcodes with equal probabilities and concatenate it with a 6 nt random barcode (as in FIG. 15A).

2. Assign this pair-end read to a type of cDNAs from the list of [linkerOnly, NoLinker, RNA1-linker, linker-RNA2, RNA1-linker-RNA2] with probability [0.1, 0.3, 0.1, 0.3, 0.2], respectively (as in FIG. 15C).

3. If this read-pair was assigned to a linker-containing type, randomly choose 1 or 2 linkers with equal probability. It is noted that a small percentage of linker-containing read-pairs contained 2 linkers; the use of equal probability was a conservative choice for estimating worst cases.

4. Generate the sequences for the RNA1 and the RNA2 parts, according to the cDNA type determined in Step 2. For both RNA1 and RNA2,
a. simulate its length from l˜Unif (15,150),
b. choose an RNA type from [“miRNA”, “mRNA”, “lincRNA”, “snoRNA”, “snRNA”, “tRNA”] based on the following probabilities:
c. if length l<50, use [0.2,0.2,0.1,0.2,0.2,0.1],
d. otherwise, use [0.05,0.4,0.2,0.2,0.1,0.05];
e. randomly choose an RNA according to the sampled RNA type from Ensembl (release 67, mouse NCBIM37),
f. randomly take a sequence segment with length/from the chosen RNA.

5. Concatenate the barcodes, linker, and RNA fragments generated from Steps 1, 3, 4, producing a synthetic cDNA sequence.

6. If the synthetic cDNA in Step 5 is 100 bp or longer, take the 100 bases from the two ends of the synthetic cDNA in forward and reverse strands respectively.

7. If the synthetic cDNA in Step 5 is shorter than 100 bp, assign its forward and reverse strands as the forward and the reverse reads, and concatenate P5 and P7 primer sequences to the two reads.

8. Simulate sequencing errors with a rate of 0.01 on each base (N. J. Loman et al., Performance comparison of benchtop high-throughput sequencing platforms. Nature biotechnology 30, 434 (May, 2012).

Steps 1-5 simulated a cDNA sequence according the experimental procedure, and steps 6-8 simulated a pair-end read based on this cDNA sequence. The simulated interacting RNA pairs, as well as the cDNA type and the length of each part (RNA1, linker, and RNA2, if applicable) were kept for comparison with the computational predictions.

### Evaluation of Intermediate and Final Results.

The synthetic data was used to evaluate the sensitivities and specificities of two intermediate analysis steps, as well as the final predictions.

First, the program-identified cDNA lengths were compared (output of Step 3 of RNA-HiC-Tools) to the actual (synthesized) lengths (Table 8). This step “3. Recovering the cDNAs in the sequencing library” assigns each cDNA into four types with respect to their lengths, namely Type 1 (<100 bp); Type 2 (100-200 bp); Type 3 (>200 bp); Type 4 (unknown). The algorithm achieved high sensitivity and specificity for identifying each type. Only very few (0.58%) of the cDNAs shorter than 200 bp were identified as longer than 200 bp. These errors were due to a small overlap (typically between 0 and 5 bps) of the forward and the reverse reads, which were not detected by the local alignment.

TABLE 8
A comparison of the program-identified and true cDNA length ranges. The counts
of program identified cDNAs of each type (Columns 1-4) are compared to their true types
(rows).

| | Identified | | | | | |
|---|---|---|---|---|---|---|
| True | Type 1 | Type 2 | Type 3 | Type 4 | Sensitivity | Specificity |
| Type 1 | 312,411 | 24 | — | — | 99.99% | 99.97% |
| Type 2 | 65 | 480,835 | 5,750 | 898 | 98.62% | 99.73% |
| Type 3 | 126 | 1,322 | 197,716 | 853 | 98.84% | 99.28% |

When the program identified length was shorter than 200 bp (Types 1 and 2), the exact length could be computed. In these cases, the program identified lengths often precisely matched the lengths of the simulated cDNAs (FIG. 33 A).

Next, the program identified chimeric configuration of each cDNA and they were compared (output of Step 4 of RNA-HiC-Tools) with the synthesized configuration. In Step “4. Parsing the chimeric cDNAs”, the algorithm assigned the cDNAs into five categories, based on the presence of the linker sequence. The algorithm reached 99.89% sensitivity and 95.82% specificity for the cDNAs in the “RNA1-linker-RNA2” form (Table 9).

TABLE 9
A comparison of the program identified and true cDNA configurations.
The counts of cDNAs of the program identified configurations (columns)
are compared to their tru configurations (rows).

| | Identified | | | | |
|---|---|---|---|---|---|
| | | | | | R1- |
| | | | | Linker- | linker- |
| True | NoLinker | LinkerOnly | R1-linker | R2 | R2 |
| NoLinker | 266,554 | 10 | — | — | 33,402 |
| LinkerOnly | — | 100,230 | — | — | — |
| R1-linker | 24 | 25 | 100,267 | — | — |
| Linker-R2 | 50 | 58 | — | 299,180 | — |
| R1-linker-R2 | 57 | 116 | 24 | 22 | 199,981 |

Lastly, the program identified and the simulated RNA-RNA interactions, which were compared. The simulated dataset contained 200,200 chimeric RNA pairs, among which 131,571 pairs of RNAs were detected (sensitivity=65.72%, specificity=92.57%). The sensitivity and specificity for interactions of each type of RNAs were also separately calculated (FIG. 33C). Regardless of the types of participating RNAs, the method showed few false positives (specificity ≥90%). Interactions that did not involve transposon RNA or snRNA exhibited fewer false negatives than those that did. This was due to the repetitive nature of transposon and snRNA sequences. The worst cases involved LINE RNAs, where sensitivities dropped to 52%. It was conservatively estimated that about a half of the interactions involving transposon RNAs could have been missed by this procedure. It was estimated that about ⅔ to ¾ of the interactions that do not involve transposon RNAs would have been identified.

### Validation by RAP-Seq.

A Malat1 RAP-sequencing experiment on mouse ES cell was carried out. After cross-linking, five antisense oligonucleotides were used to pulldown Malat1 and then sequence the other RNAs that were purified together with Malat1. Actin RAP-sequencing was performed as the control. Malat1 RNA itself exhibited a 5.81 fold increase in Malat1 RAP-seq than Actin RAP-seq, confirming the validity of the purification. RNA Hi-C reported that Malat1 as a “hub” lincRNA which interacted with Tfrc, S1c2a3, Eif4a2, and 0610007P14Rik RNA. These RNAs showed 14.6 (0610007P14Rik), 4.53 (S1c2a3), 3.38 (Eif4a2), and 2.39 (Tfrc) fold increase in Malat1 RAP-seq than Actin RAP-seq (the largest Chi-square test p-value <0.0003). This suggests a strong overlap of Malat1 targets from RNA Hi-C and Malat1 RAP-seq.

For another validation, a Tfrc RAP-seq experiment was performed. Tfrc was identified as a Malat1 interacting RNA from RNA Hi-C (FIG. 1D). It was asked whether Tfrc pulldown could reversely identify Malat1. The Tfrc RNA itself showed 2.87 fold of increase in Tfrc RAP-seq compared to Actin RAP-seq. In the same dataset, Malat1 RNA showed 3.84 fold increase, comparing Tfrc RAP-seq to Actin RAP-seq (p-value <2.2×10⁻¹⁶, derived from testing the null hypothesis fold change=1).

The other RNAs interacting with Tfrc as identified by RNA Hi-C was checked and could be validated by Tfrc RAP-seq as well. RNA Hi-C data identified a total of five RNAs as interacting with Tfrc. Besides Malat1, the other four were all snoRNAs, namely Snord13, SNORA3, Snord52, SNORA74. Three of these 4 snoRNAs exhibited fold increases (1.4 fold for Snord13, 13.6 fold for SNORA3, 8.7 fold for SNORA74) in Tfrc RNA-seq as compared to Actin RAP-seq, confirming these interactions (Chi-square test p value <0.00002). In summary, RAP-seq confirmed nearly all RNA Hi-C identified interactions. With the two types of experiments (RNA Hi-C and RAP-seq), a few RNA interactions (mentioned above) were nominated as “real” in mouse ES cells.

Comparison of snoRNA-mRNA Interactions with mRNA Pseudouridines.

The pseudouridylation sequencing data (Ψ-seq) were compared with the RNA-interaction sites. Schwartz et al. carried out Ψ-seq in yeast and in mouse bone-marrow-derived dendritic cells (BMDDC). BMDDC Ψ-seq data were retrieved (CMC treated GSM1464234 and control GSM1464235), and called pseudouridines (Ψ-sites) using the bioinformatic procedure described in the paper. Briefly, Ψ-sites were determined as having more than 5 CMC-treated reads next to a ‘U’ on the correct strand and direction and having a Ψ-fc value greater than 3. This yielded 386 Ψ-sites out of a total of 8,194,131 ‘U’ positions (0.00471% ‘U’s were Ψ-sites).

Next, these 386 Ψ-sites to RNA Hi-C identified RNA interaction sites were compared. It was acknowledged that Ψ-seq and RNA Hi-C were done in different cell types. Nevertheless, within the RNA interaction sites, 93 were Ψ-sites out of a total of 551,634 ‘U’s (0.0109%). Therefore, RNA interaction sites determined by RNA Hi-C were enriched with Ψ-sites (odds ratio=4.4, Chi-square test p-value=7.70×10⁻⁹⁵).

Furthermore, it was asked whether the Ψ-sites were enriched in the snoRNA-mRNA interaction sites detected by RNA Hi-C. Within snoRNA participating interaction sites, there were 57 Ψ-sites out of a total of 136,535 ‘U’s (0.0381%). Compared to the entire transcriptome, RNA Hi-C detected snoRNA-participated interaction sites were greatly enriched with Ψ-sites (odds ratio=10.2, Chi-square test p-value <1×10⁻¹⁰⁰). Although snoRNA was known to contribute to RNA pseudouridination, these data indicate which snoRNAs may be specifically responsible. (Table 10).

TABLE 10
Two-way contingency tables for association test
of Ψ sites and RNA interaction sites.

| | | None | Total # | Odds |
|---|---|---|---|---|
| | Ψ-sites | Ψ-sites | of ‘U’s | ratio = 4.4 |
| Within RNA interaction | 93 | 551,541 | 551,634 | |
| sites as detected by | | | | |
| RNA Hi-C | | | | |
| Others | 293 | 7,642,204 | 7,642,497 | P value = |
| Total # of ‘U’s | 386 | 8,193,745 | 8,194,131 | 7.70 × 10⁻⁹⁵ |
| | | None | Total # | Odds |
| | Ψ-sites | Ψ-sites | of ‘U’s | ratio = 10.2 |
| Within snoRNA | 57 | 136,478 | 136,535 | |
| participated | | | | |
| interaction sites as | | | | |
| detected by RNA Hi-C | | | | |
| Others | 329 | 8,057,267 | 8,057,596 | P value < |
| Total # of ‘U’s | 386 | 8,193,745 | 8,194,131 | 10⁻¹⁰⁰ |

Interactions between RNA molecules exert key regulatory roles and are often mediated by RNA binding proteins (Ray, D. et al. A compendium of RNA-binding motifs for decoding gene regulation. Nature 499, 172-177, doi:10.1038/nature12311 (2013)) such as ARGONAUTE proteins (AGO), PUM2, QKI, and snoRNP proteins (Meister, G. Argonaute proteins: functional insights and emerging roles. Nat Rev Genet 14, 447-459, doi:10.1038/nrg3462 (2013); Hafner, M. et al. Transcriptome-wide identification of RNA-binding protein and microRNA target sites by PAR-CLIP. Cell 141, 129-141, doi:10.1016/j.cell.2010.03.009 (2010); Granneman, S., Kudla, G., Petfalski, E. & Tollervey, D. Identification of protein binding sites on U3 snoRNA and pre-rRNA by UV cross-linking and high-throughput analysis of cDNAs. Proceedings of the National Academy of Sciences of the United States of America 106, 9613-9618, doi:10.1073/pnas.0901997106 (2009)). Despite recent advances, such as PAR-CLIP 4, HITS-CLIP 6, and CLASH 7,8, it remains a formidable challenge to map all protein-assisted RNA-RNA interactions (Hafner, M. et al. Transcriptome-wide identification of RNA-binding protein and microRNA target sites by PAR-CLIP. Cell 141, 129-141, doi:10.1016/j.cell.2010.03.009 (2010); Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009); Helwak, A., Kudla, G., Dudnakova, T. & Tollervey, D. Mapping the human miRNA interactome by CLASH reveals frequent noncanonical binding. Cell 153, 654-665, doi:10.1016/j.cell.2013.03.043 (2013). Kudla, G., Granneman, S., Hahn, D., Beggs, J. D. & Tollervey, D. Cross-linking, ligation, and sequencing of hybrids reveals RNA-RNA interactions in yeast. Proc Natl Acad Sci USA 108, 10010-10015, doi:10.1073/pnas.1017386108 (2011)). In each of these three approaches, only the interactions mediated by one RNA-binding protein can be analyzed per experiment. HITS-CLIP and PAR-CLIP cannot directly map the interacting RNA pairs. Additionally, each experiment requires either a protein-specific antibody (HITS-CLIP or PAR-CLIP) or stable expression of a tagged protein in transformed cell lines (CLASH).

Earlier approaches often require ectopic expression of one or several components of the proposed interactions. Such methods include luciferase reporter assays and the use of synthetic RNA mimics for target capturing (Nicolas, F. E. Experimental validation of microRNA targets using a luciferase reporter system. Methods in molecular biology 732, 139-152, doi:10.1007/978-1-61779-083-6_11 (2011); Lal, A. et al. Capture of microRNA-bound mRNAs identifies the tumor suppressor miR-34a as a regulator of growth factor signaling. PLoS Genet 7, e1002363, doi:10.1371/journal.pgen.1002363 (2011)). Because ectopic expression rarely reproduces the endogenous expression levels, it is prudent to interpret the results from these methods as potential interactions rather than in vivo interactions. It is noted that the premise that miRNA tend to “promiscuously” interact with many mRNAs were primarily derived from data using ectopic expression (Du, T. & Zamore, P. D. Beginning to understand microRNA function. Cell Res 17, 661-663, doi: 10.1038/cr.2007.67 (2007)).

The RNA Hi-C method was developed to detect protein-assisted RNA-RNA interactions in vivo. In this procedure, RNA molecules are cross-linked with their bound proteins then ligated to a biotinylated RNA linker such that RNA molecules co-bound by the same protein form a chimeric RNA of the form RNA1-Linker-RNA2. These linker-containing chimeric RNAs are isolated using streptavidin coated magnetic beads and subjected to pair-end sequencing (Methods, FIG. 1A, FIGS. 7A to 7B). Thus, each non-redundant pair-end read reflects a molecular interaction. Some design aspects of this technology were inspired by chromosome conformation capture methods (Kalhor, R., Tjong, H., Jayathilaka, N., Alber, F. & Chen, L. Genome architectures revealed by tethered chromosome conformation capture and population-based modeling. Nature biotechnology 30, 90-98, doi:10.1038/nbt.2057 (2012); Belton, J. M. et al. Hi-C: a comprehensive technique to capture the conformation of genomes. Methods 58, 268-276, doi: 10.1016/j.ymeth.2012.05.001 (2012)).

The RNA Hi-C method offers several advantages for mapping RNA-RNA interactions. First, RNA Hi-C directly analyzes the endogenous cellular features without introducing any exogenous nucleotides or protein-coding genes prior to cross-linking (Hafner, M. et al. Transcriptome-wide identification of RNA-binding protein and microRNA target sites by PAR-CLIP. Cell 141, 129-141, doi:10.1016/j.cell.2010.03.009 (2010); Helwak, A., Kudla, G., Dudnakova, T. & Tollervey, D. Mapping the human miRNA interactome by CLASH reveals frequent noncanonical binding. Cell 153, 654-665, doi:10.1016/j.cell.2013.03.043 (2013); Lal, A. et al. Capture of microRNA-bound mRNAs identifies the tumor suppressor miR-34a as a regulator of growth factor signaling. PLoS Genet 7, e1002363, doi:10.1371/journal.pgen.1002363 (2011); Baigude, H., Ahsanullah, Li, Z., Zhou, Y. & Rana, T. M. miR-TRAP: a benchtop chemical biology strategy to identify microRNA targets. Angew Chem Int Ed Engl 51, 5880-5883, doi:10.1002/anie.201201512 (2012)). This eliminates the uncertainty of reporting spurious interactions produced by changing the RNA or protein expression levels. Moreover, it makes RNA Hi-C well suited for assaying tissue samples. Second, the use of a biotinylated linker as a selection marker circumvents the requirement for a protein-specific antibody or the need to express a tagged protein. This allows for an unbiased mapping of the RNA-RNA interactome. As described in the literature other methods can only work with one RNA-binding protein at a time. Third, only RNA brought together by the same, singular protein molecule are captured, avoiding capture of independent RNA molecules that are individually bound to different copies of the same protein (potentially leading to reporting spurious interactions) (Hafner, M. et al. Transcriptome-wide identification of RNA-binding protein and microRNA target sites by PAR-CLIP. Cell 141, 129-141, doi:10.1016/j.cell.2010.03.009 (2010); Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009)). Fourth, false positives that result from RNAs ligating randomly to other nearby RNAs are minimized by performing the RNA ligation step on streptavidin beads in extremely dilute conditions. Fifth, the RNA linker provides a clear boundary delineating sequencing reads that span across the ligation site, thus avoiding ambiguities in mapping the sequencing reads. Sixth, potential PCR amplification biases are removed by attaching a random 6 nucleotide barcode to each chimeric RNA before PCR amplification and subsequently counting completely overlapping sequencing reads with identical barcodes only once (Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009), Loeb, G. B. et al. Transcriptome-wide miR-155 binding map reveals widespread noncanonical microRNA targeting. Mol Cell 48, 760-770, doi:10.1016/j.molcel.2012.10.002 (2012); Wang, Z. et al. iCLIP predicts the dual splicing effects of TIA-RNA interactions. PLoS Biol 8, e1000530, doi:10.1371/journal.pbio.1000530 (2010); Konig, J. et al. iCLIP reveals the function of hnRNP particles in splicing at individual nucleotide resolution. Nat Struct Mol Biol 17, 909-915, doi:10.1038/nsmb.1838 (2010)).

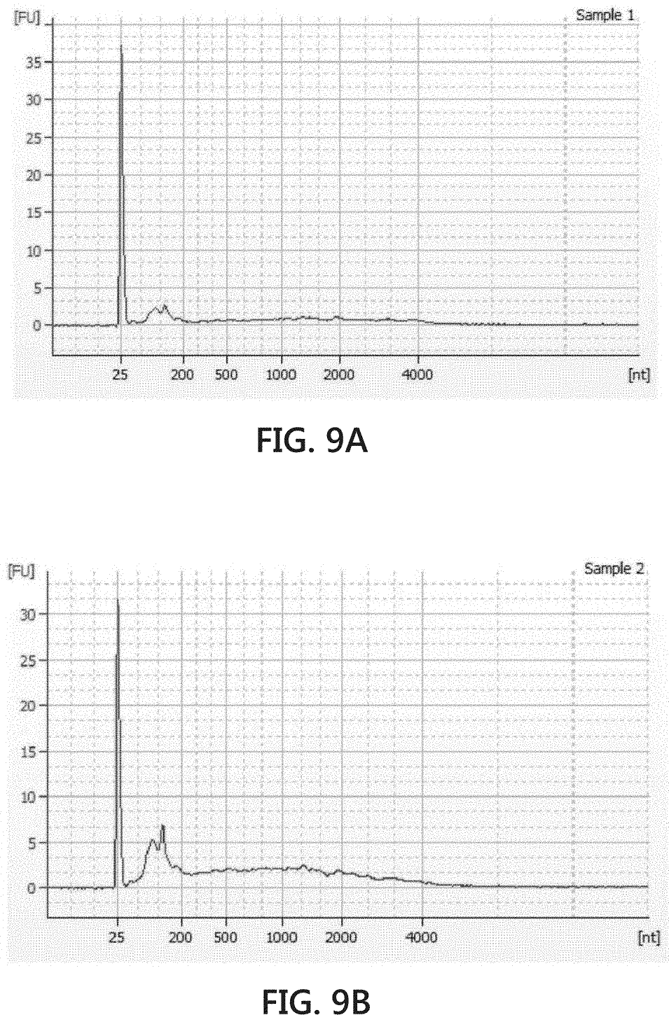

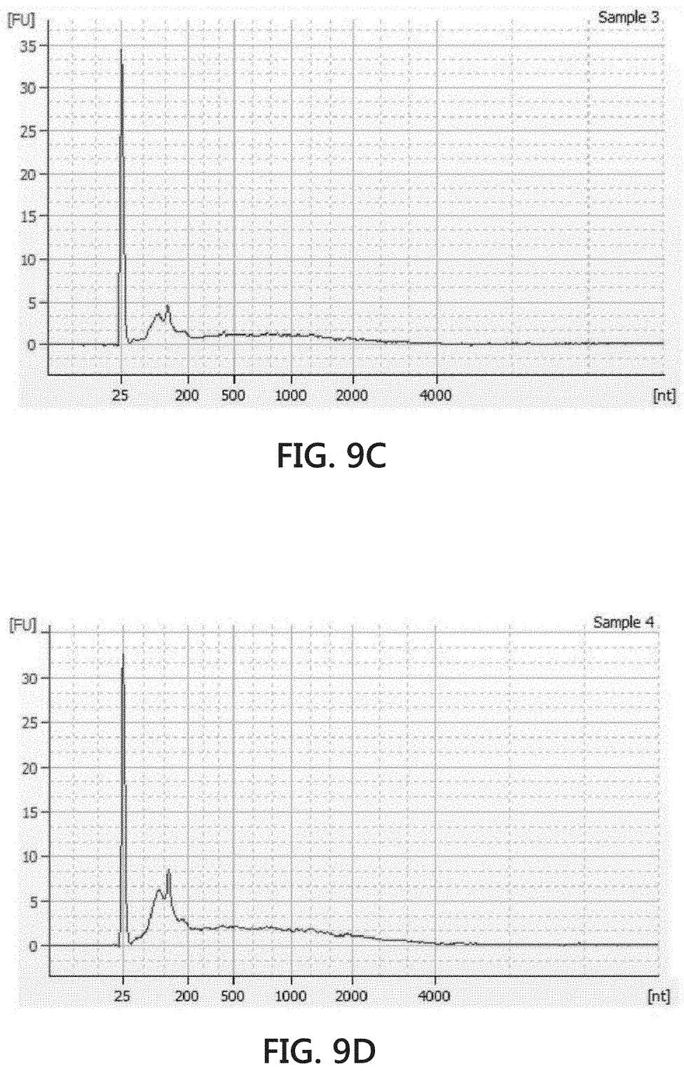

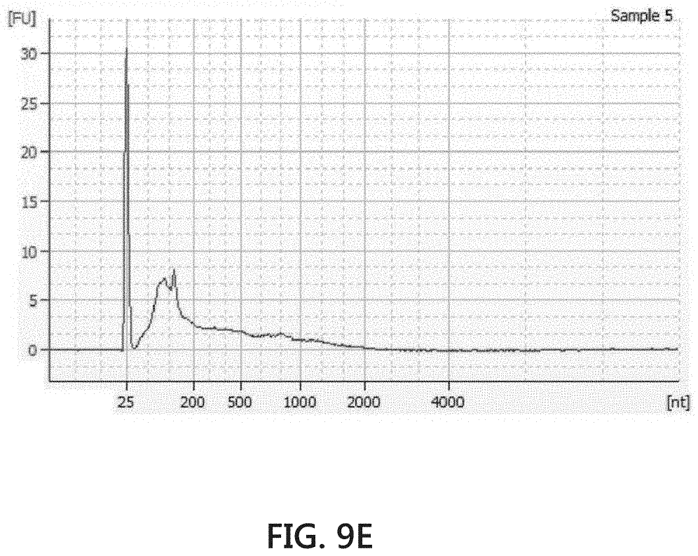

Two independent RNA Hi-C assays were carried out on mouse embryonic stem (ES) cells with minor technical differences (Table 5, FIGS. 9A to 9B and 10-12), which were designated as ES-1 and ES-2. A library for indirect RNA interactions was produced using two cross-linking agents (formaldehyde and EGS) which “effectively captures RNAs linked indirectly through multiple protein intermediates” 1 (ES-indirect) (Engreitz, J. M. et al. RNA-RNA interactions enable specific targeting of noncoding RNAs to nascent Pre-mRNAs and chromatin sites. Cell 159, 188-199, doi:10.1016/j.cell.2014.08.018 (2014); Nowak, D. E., Tian, B. & Brasier, A. R. Two-step cross-linking method for identification of NF-kappaB gene network by chromatin immunoprecipitation. Biotechniques 39, 715-725 (2005); Zeng, P. Y., Vakoc, C. R., Chen, Z. C., Blobel, G. A. & Berger, S. L. In vivo dual cross-linking for identification of indirect DNA-associated proteins by chromatin immunoprecipitation. BioTechniques 41, 694-698 (2006); Zhao, J. et al. Genome-wide identification of polycomb-associated RNAs by RIP-seq. Mol Cell 40, 939-953, doi:10.1016/j.molcel.2010.12.011 (2010)). Two other unique libraries were produced from mouse embryonic fibroblasts (MEF) and mouse brain, offering two additional datasets for bioinformatics quality assessment (FIGS. 13A to 13C). It was confirmed that each library contained RNA constructs of the desired form (RNA1-Linker-RNA2) and lengths (FIG. 1B). Each library was sequenced to yield, on average, 47.3 million pair-end reads, among which approximately 15.1 million non-redundant pair-end reads represented the desired chimeric form (FIG. 1C). Additionally, three control experiments were carried out. The first and the second control experiments excluded the cross-linking step (non-cross-linking control) and the protein biotinylation step (non-biotinylation control), respectively (Control experiments for RNA Hi-C). The third control experiment used Drosophila S2 cells and mouse ES cells to test the extent of random ligation of RNAs (cross-species control). After cross-linking, the lysates from the two cell lines were mixed before protein biotinylation and proximity ligation. The mixture was subjected to the rest of the experimental procedure and resulted in a sequenced library (Fly-Mm). The proportion of RNA pairs mapped to two species (false positives) is 0.52%. However, when the ES-1 sequencing library was subjected to the same informatics analysis, 0.55% RNA pairs were mapped to two species (mouse and the fly genomes), suggesting that the experimental false positives (supposedly due to random ligation) were less frequent than the error range of the informatics procedure (Control experiments for RNA Hi-C).

TABLE 5
Description of the RNA Hi-C samples. The “total # of read pairs”
is the number of pair-end sequencing reads for each sample. The “# of
non-duplicate read pairs in the form of RNA1-Linker-RNA2” is the
number of the pair-end reads in the output of Step 4, parsing the chimeric
cDNAs, of the bioinformatics pipeline.

| Sample name | ES-1 | ES-2 | ES-indirect | MEF | Brain |
|---|---|---|---|---|---|
| Cell type | ES cells | ES cells | ES cells | MEF | Brain Tissue |
| Crosslinking | 254 nm UV | 254 nm UV | Dual crosslinking | 254 nm UV | 254 nm UV |
| RNA-protein | Direct | Direct | Indirect | Direct | Direct |
| interactions | | | | | |
| Protein | Detergents | Detergents | Sonication | Detergents | Detergents |
| solubilization | | | | | |
| First | 1000-2000 nt | ~1000 nt | ~1000 nt | ~300 nt | ~1000 nt |
| fragmentation | | | | | |
| rRNA removal | Duplex-specific | Antibody | Duplex-specific | Antibody | Duplex-specific |
| | nuclease | based | nuclease | based | nuclease |
| Sample | ACCT | GGCG | AATG | GGCG | TTGT |
| barcode | | | | | |
| Total # of | 45,702,794 | 49,316,127 | 74,009,386 | 83,083,324 | 36,463,565 |
| read pairs | | | | | |
| # of non- | 13,848,413 | 9,553,722 | 19,554,316 | 17,616,980 | 2,877,233 |
| duplicate read | | | | | |
| pairs in the | | | | | |
| form of RNA1- | | | | | |
| Linker-RNA2 | | | | | |

A suite of bioinformatics tools was created (RNA-HiC-tools) to analyze and visualize RNA Hi-C data (FIGS. 14 and 15A to 15F). RNA-HiC-tools automated the analysis steps, including removing PCR duplicates, splitting multiplexed samples, identifying the linker sequence, splitting junction reads, calling interacting RNAs, performing statistical assessments, categorizing RNA interaction types, calling interacting sites, and analyzing RNA structure (Methods). It also provides visualization tools for both the RNA-RNA interactome and the proximal sites within an RNA (FIGS. 16A to 16C).

The five RNA Hi-C libraries were compared. ES-1 and ES-2 were most similar judged by correlations of FPKMs (separately calculated for the read fragments on the left and the right sides of the linker), followed by ES-indirect, and then MEF and brain tissue (FIGS. 13A to 13C). The interacting RNA pairs identified from ES-1 and those from ES-2 exhibited strong overlaps (p-value <10-35, permutation test) (Table 6). The interactions identified in MEF did not exhibit significant overlaps with those in either of the ES samples (p-value for each overlap=1, permutation tests). For example, an interaction between the 3′ UTR of Trim25 RNA and small nucleolar RNA (snoRNA) Snora1 was supported by 24 and 22 pair-end reads in ES-1 and ES-2 samples, respectively, but was not detected in ES-indirect (Differences between dual cross-linking and UV cross-linking) or MEF libraries (FIG. 1C). Including Snora1, as many as 172 snoRNAs were identified as having interacted with mRNAs detected in AGO HITS-CLIP data (green lane, FIG. 1C) and enzymatically processed small RNAs (red lane, FIG. 1C, FIGS. 17A to 17D, 18 and 19A to 19C) (Yu, P. et al. Spatiotemporal clustering of the epigenome reveals rules of dynamic gene regulation. Genome Res 23, 352-364, doi:10.1101/gr.144949.112 (2013).). This supports the proposition that transcripts from snoRNA genes could be enzymatically processed into miRNA-like small RNAs and interact with mRNAs in RISC complex (Ender, C. et al. A human snoRNA with microRNA-like functions. Mol Cell 32, 519-528, doi:10.1016/j.molcel.2008.10.017 (2008); Brameier, M., Herwig, A., Reinhardt, R., Walter, L. & Gruber, J. Human box C/D snoRNAs with miRNA like functions: expanding the range of regulatory RNAs. Nucleic Acids Res 39, 675-686, doi:10.1093/nar/gkq776 (2011)). (Other RNAs with miRNA-like interactions).

TABLE 6
The distribution of read pairs mapped to two genomes. The reads
not included in this table were either not mappable to any genome
or having the same RNA part mapped to both genomes. An RNA part
is the read sequence on either side of the linker sequence.

| | Both RNA | Both RNA | One part mapped to |
|---|---|---|---|
| | parts mapped | parts mapped | mouse and the other |
| | mouse genome | to fly genome | part mapped to fly |
| Total: | 3,102,147 | 5,338,431 | 44,229 |
| 8,484,807 | | | |
| RNA1-RNA2 | | | |
| pairs | | | |

The ES-1 and ES-2 libraries were merged to infer the RNA-RNA interactome in ES cells. This data included 4.54 million non-duplicated pair-end reads that were unambiguously split into two RNA fragments with both fragments uniquely mapping to the genome (mm9). 46,780 inter-RNA interactions were identified (FDR<0.05, Fisher's exact test with Benjamin & Hochberg correction) (FIG. 20). As expected, the RNA expression level (FPKM) is weakly correlated with the number of RNA Hi-C reads on each RNA, but FPKM is not correlated with the statistical significance (FDR) of the interactions (FIGS. 20C-20D). mRNA-snoRNA interactions were the most abundant type, although thousands of mRNA-mRNA and hundreds of lincRNA-mRNA, pseudogeneRNA-mRNA, miRNA-mRNA interactions were also detected (FIG. 21). This is the first RNA-RNA interactome described in any organism. Our simulation suggested approximately 66% sensitivity and 93% specificity for the entire experimental and analysis procedure (Simulation analysis of RNA Hi-C).

In order to confirm interactions at a larger scale, RNA antisense oligonucleotide purification sequencing was carried out (RAP-seq)(Engreitz, J. M. et al. RNA-RNA interactions enable specific targeting of noncoding RNAs to nascent Pre-mRNAs and chromatin sites. Cell 159, 188-199, doi:10.1016/j.cell.2014.08.018 (2014)). First, Malat1 RAP-seq and Actb RAP-seq (control) was performed to test the interactions involving Malat1 (Comparison of snoRNA-mRNA interactions with mRNA pseudouridines). Malat1 RNA itself exhibited a 5.81 fold increase in Malat1 RAP-seq over Actb RAP-seq, confirming the validity of the purification. The RNA-Hi C reported Malat1 interacting RNAs (FIG. 1D) showed 14.6 (0610007P14Rik), 4.53 (S1c2a3), 3.38 (Eif4a2), and 2.39 (Tfrc) fold increase in Malat1 RAP-seq over Actb RAP-seq (p-value <0.0003, Chi-square test). This suggests a strong overlap of Malat1 targets in RNA Hi-C and Malat1 RAP-seq. Next, it was asked whether Tfrc RAP could reversely identify Malat1 by Tfrc RAP-seq (Comparison of snoRNA-mRNA interactions with mRNA pseudouridines). The Tfrc RNA itself showed 2.87 fold of increase in Tfrc RAP-seq compared to Actb RAP-seq. Malat1 exhibited 3.84 fold increase (p-value <2.2×10-16, derived from testing the null hypothesis fold change=1). In addition, three out of four other Tfrc interacting RNAs identified by RNA Hi-C exhibited 1.4-13.6 fold increases (p value <0.00002, Chi-square test). Taken together, 7 additional RNA Hi-C identified interactions were validated by RAP-seq.

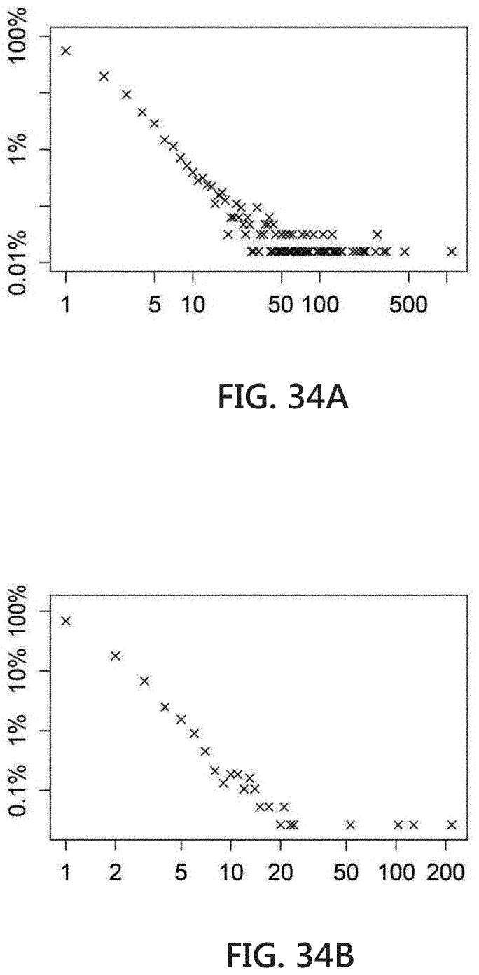

RNA-RNA interactions have been reported as “surprisingly promiscuous” (Du, T. & Zamore, P. D. Beginning to understand microRNA function. Cell Res 17, 661-663, doi:10.1038/cr.2007.67 (2007)). It was suggested that each miRNA interacts with 300 to 1,000 mRNAs in one cell type, and a similar picture was proposed for lincRNAs (Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009); Guttman, M. et al. Chromatin signature reveals over a thousand highly conserved large non-coding RNAs in mammals. Nature 458, 223-227, doi:10.1038/nature07672 (2009)). However, the observed RNA-RNA interactome (46,780 interactions) is a scale-free network, with a degree distribution conforming to power law (FIG. 1D, FIGS. 34A to 34B) (Barabasi, A. L. & Oltvai, Z. N. Network biology: understanding the cell's functional organization. Nat Rev Genet 5, 101-113, doi:10.1038/nrg1272 (2004)). In other words, the majority of RNAs that participated in RNA-RNA interactions have specific interaction partners, and the quantity of RNAs with a given number of interaction partners decreases exponentially as that number of interaction partners increases. This global property does not change if the interactions are restricted to only of mRNAs, lincRNAs, miRNAs, pseudogene RNAs, and antisense transcripts (FIG. 1D). Moreover, the RNA-RNA interactome derived from mouse brain (57,833 interactions) is scale-free (FIG. 34B), suggesting this global property is not cell-type specific. In each cell type, the vast majority of the miRNAs and lincRNAs interacted with 1 to 3 mRNAs, more than 80% of which were specifically interacting with one mRNA (FIG. 1E). In summary, “promiscuous” RNAs are exceptions in the RNA-RNA interactomes derived from RNA Hi-C. It is speculated that this is because, unlike previous methods, RNA Hi-C directly captured the RNA molecules co-attached to each individual protein molecule in the endogenous cellular condition.

The majority (83.05%) of the interacting RNAs exhibited overlapping RNA Hi-C reads (FIG. 3A), suggesting interactions were often concentrated at specific segments of an RNA. “Peaks” of overlapping read fragments were identified and termed “interaction sites” (FIG. 3B). Interaction sites appeared not only on miRNAs (the entire mature miRNA), mRNAs, lincRNAs, but also on pseudogene and transposon RNAs (FIG. 3C). Over 2000 interaction sites were harbored in L1, SINE, ERVK, MaLR, and ERV1 transposon RNAs (Table 7), indicative of their frequent interactions with other RNAs (Shalgi, R., Pilpel, Y. & Oren, M. Repression of transposable-elements—a microRNA anti-cancer defense mechanism? Trends in genetics: TIG 26, 253-259, doi: 10.1016/j.tig.2010.03.006 (2010); Yuan, Z., Sun, X., Liu, H. & Xie, J. MicroRNA genes derived from repetitive elements and expanded by segmental duplication events in mammalian genomes. PloS one 6, e17666, doi:10.1371/journal.pone.0017666 (2011)). Additionally, pseudouridines were enriched in the mRNA interactions sites of snoRNA-mRNA interactions, corroborating the idea that some RNA segments were favored in certain types of RNA interactions (Schwartz, S. et al. Transcriptome-wide mapping reveals widespread dynamic-regulated pseudouridylation of ncRNA and mRNA. Cell 159, 148-162, doi:10.1016/j.cell.2014.08.028 (2014)).

TABLE 7
Distribution of interaction sites in different types of genes
and transposons. Novel: unannotated genomic regions.

| | | Number of | | Total copy |
|---|---|---|---|---|
| | Number of | genes | Total number | number of |
| | interaction | containing | of genes in | genes in |
| Type | sites | these sites | the genome | the genome |
| mRNA | 12439 | 6600 | 22562 | 22562 |
| snoRNA | 553 | 511 | 1561 | 1561 |
| tRNA | 365 | 57 | 60 | 4760 |
| lincRNA | 363 | 243 | 2054 | 2054 |
| snRNA | 226 | 13 | 32 | 1429 |
| miRNA | 27 | 25 | 1630 | 1630 |
| misc_RNA | 33 | 17 | 114 | 487 |
| pseudogene | 234 | 131 | 5306 | 5306 |
| antisense | 34 | 31 | 1351 | 1351 |
| LINE (L1) | 726 | 76 | 112 | 884320 |
| LINE (L2) | 26 | 4 | 4 | 65481 |
| LTR (ERVK) | 346 | 96 | 150 | 245391 |
| LTR (MaLR) | 274 | 60 | 102 | 430745 |
| LTR (ERV1) | 235 | 39 | 113 | 61660 |
| LTR (ERVL) | 78 | 31 | 88 | 111531 |
| SINE | 458 | 32 | 40 | 1521108 |
| Novel | 4426 | | | |

It was asked whether base complementation is utilized by different types of RNA-RNA interactions. It was estimated the hybridization energy of a pair of interacting RNAs by the average hybridization energy of the pairs of ligated fragments (RNA1, RNA2), and compared it to the hybridization energy of control RNAs generated by random shuffling of the bases (Ray, D. et al. A compendium of RNA-binding motifs for decoding gene regulation. Nature 499, 172-177, doi:10.1038/nature12311 (2013); Bellaousov, S., Reuter, J. S., Seetin, M. G. & Mathews, D. H. RNAstructure: web servers for RNA secondary structure prediction and analysis. Nucleic Acids Research 41, W471-W474, doi:Doi 10.1093/Nar/Gkt290 (2013)). Complementary bases were preferred in nearly all types of RNA-RNA interactions, and were most pronounced in transposonRNA-mRNA, mRNA-mRNA, pseudogeneRNA-mRNA, lincRNA-mRNA, miRNA-mRNA interactions (p-values <2.4-18), but was not observed in LTR-pseudogeneRNA interactions (FIG. 3D, FIGS. 24A to 24F). This data suggests a new mechanism, where base pairing facilitates sequence-specific posttranscriptional regulation in long RNAs.

If these RNA-RNA interactions are sequence-specific, the RNA interaction sites should be under selective pressure (Gong, C. & Maquat, L. E. lncRNAs transactivate STAU1-mediated mRNA decay by duplexing with 3′ UTRs via Alu elements. Nature 470, 284-288, doi:10.1038/nature09701 (2011)). It was found that the interspecies conservation levels are strongly increased at the interaction sites, and the peak of conservation precisely pinpointed the junction of the two RNA fragments (FIG. 3D) (Cooper, G. M. et al. Distribution and intensity of constraint in mammalian genomic sequence. Genome Res 15, 901-913, doi:10.1101/gr.3577405 (2005)). When interacting with lincRNAs, pseudogene RNAs, transposon RNAs, or other mRNAs, the interaction sites on mRNAs were more conserved than the rest of the transcripts (FIGS. 25A to 25C). The interactions sites on lincRNAs and pseudogene RNAs exhibited increased conservation in lincRNAs-mRNA, pseudogeneRNA-mRNA, and pseudogeneRNA-transposonRNA interactions (FIGS. 25A to 25C). The increased conservation on interaction sites was not due to exon-intron boundaries (FIG. 26). Taken together, base complementation is wide-spread in the interactions of long RNAs. The complementary regions are evolutionarily conserved.

Although designed RNA Hi-C were originally for mapping inter-molecule interactions, it was found that RNA Hi-C revealed RNA secondary and tertiary structures. All the analyses above were based on inter-molecular reads. By looking at intra-molecular reads, two characteristics of RNA structure were learned. First, the footprint of single stranded regions of an RNA were identified by the density of RNase I digestion sites (RNase I digestion was applied before ligation, see Step 2 in FIG. 1A, FIGS. 27A to 27D). Second, the spatially proximal sites of each RNA were captured by proximity ligation (Step 5 in FIG. 1A). A total of 67,221 read pairs were mapped to individual genes, but were not mapped within 2,000 bp of each other or on the same strand, and thus were generated from intra-molecule cutting and ligation (FIGS. 28A to 28C). Each cut-and-ligated sequence can be unambiguously assigned to one of two structural classes by comparing the orientations of RNA1 and RNA2 in the sequencing read with their orientations in the genome (FIG. 4A). These reads provided spatial proximity information for 2,374 RNAs, including those from 1,696 known genes and 678 novel genes. For example, 277 cut-and-ligated sequences were produced from Snora73 transcripts (FIG. 4B). The density of RNase I digestion sites (FIG. 4C) was strongly predictive of the single stranded regions of the RNA (heatmap, FIG. 4E). Six pairs of proximal sites were detected (circles, FIG. 4D). Each pair was supported by three or more cut-and-ligated sequences with overlapping ligation positions (black spots, FIG. 4B). Five out of the six proximal site pairs were physically close in the generally accepted secondary structure (arrows of the same color, FIG. 4E). On Snora14, a pair of inferred proximal sites appeared distant, according to sequenced inferred secondary structure (, FIGS. 29A to 29B). However, ribonucleoprotein DYSKERIN bent Snora14 transcript in vivo, making the two pseudouridylation loops close to each other, as predicted by the cut-and-ligated sequence (arrows, FIG. 4F) (Kiss, T., Fayet-Lebaron, E. & Jady, B. E. Box H/ACA small ribonucleoproteins. Mol Cell 37, 597-606, doi:10.1016/j.molcel.2010.01.032 (2010)). Structural information can even be derived on novel transcripts and some parts of mRNAs (FIGS. 30A to 30C and 31). To date, resolving the spatially proximal bases of any individual RNA remains a grand challenge. RNA Hi-C in ES cells provides intra-molecule spatial proximity information for the thousands of RNAs. Additionally, the single strand footprints of every RNA are mapped at the same time. Thus, RNA Hi-C largely expanded our capacity to examine RNA structures.

The key to mapping RNA interactions is selection. The introduction of a selectable linker in RNA Hi-C enabled an unbiased selection of interacting RNAs, making it possible to globally map an RNA-RNA interactome. The number of interacting partners per RNA in ES cells was strongly unbalanced, resulting in a scale-free RNA network. Interactions between long RNAs frequently used a small fraction of the transcripts. Analogous to protein interaction domains, the notion of RNA interaction sites were proposed. RNA interaction sites utilized base pairing to facilitate interactions of long RNAs, suggesting a new type of trans regulatory sequences. These trans regulatory sequences are more evolutionarily conserved than other parts of transcripts. RNA structure could be mapped by RNA Hi-C as well. Here an example is provided where an RNA was bent by a protein, and such tertiary structure was revealed by the intro-molecule reads of RNA Hi-C. This method and data should greatly facilitate future investigations of RNA functions and regulatory roles.

Software Access

The RNA-HiC-tools software is available at http://systemsbio.ucsd.edu/RNA-Hi-C.

From the foregoing, it will be appreciated that various embodiments of the present disclosure have been described herein for purposes of illustration, and that various modifications can be made without departing from the scope and spirit of the present disclosure. Accordingly, the various embodiments disclosed herein are not intended to be limiting, with the true scope and spirit being indicated by the following claims.

### Additional Embodiments

In some embodiments, a method for generating chimeric RNAs comprises RNAs which interact with one another in a cell, wherein the method comprises cross-linking RNA to protein and ligating RNAs cross-linked to the same protein molecule together to form a chimeric RNA. In some embodiments, said cross-linking of RNA to protein is performed on an intact cell or in a cell lysate. In some embodiments, said cross-linking comprises UV cross-linking. In some embodiments, the method further comprises associating said protein with an agent which facilitates immobilization of said protein on a surface. In some embodiments, said agent which facilitates immobilization comprises biotin. In some embodiments, the protein is biotinylated at least one cysteine. In some embodiments, the method further comprises fragmenting said RNAs cross-linked to the same protein molecule. In some embodiments, said fragmenting comprises contacting said RNAs cross-linked to the same protein molecule with an RNAse under conditions which facilitate partial digestion of said RNAs. In some embodiments, the method further comprises linking said RNAs cross-linked to the same protein molecule to an agent which facilitates recovery of said RNAs. In some embodiments, said linking comprises ligating the ends of said RNAs to said agent. IN some embodiments, the RNA is ligated with a biotin-tagged RNA linker. In some embodiments, the biotin-tagged RNA linker is 2, 3, 4, 5, 6, 7, 8, 9, 10, 11, 12, 13, 14, 15, 16, 17, 18. 19, 20, 21, 22, 23, 24, 25, 26, 27, 28, 29 or 30 nucleotides long or any length between any aforementioned values. In some embodiments, said agent which facilitates recovery of said RNAs comprises a nucleic acid. In some embodiments, said nucleic acid comprises a nucleic acid having biotin thereon. In some embodiments, said linking of said nucleic acid having biotin thereon to said ends of said RNAs comprises ligating said nucleic acid having biotin thereon to the 5′ ends of said RNAs prior to ligating said RNAs cross-linked to the same protein molecule together to form a chimeric RNA. In some embodiments, the method further comprises removing said biotin from the 5′ region of said chimeric RNA. In some embodiments, the method further comprises recovering said chimeric RNAs. In some embodiments, the method further comprises fragmenting said chimeric RNAs. In some embodiments, the method further comprises DNAse treatment to eliminate DNA contamination. In some embodiments, said fragmenting of said chimeric RNAs comprises contacting said chimeric RNAs with an RNAse under conditions which facilitate partial digestion of said RNAs. In some embodiments, the method further comprises reverse transcribing said chimeric RNAs to generate a chimeric cDNA. In some embodiments, the method further comprises determining at least a portion of the sequences in said chimeric RNAs or chimeric cDNAs which originate from each of the RNAs in said chimeric RNAs or chimeric cDNAs. In some embodiments, the method further comprises identifying the RNAs present in said chimeric RNAs, thereby identifying RNAs which interact with one another in a cell. In some embodiments, at least 100, at least 500, at least 1000 or more than 1000 RNA-RNA interactions in the cell are identified. In some embodiments, substantially all of the RNAs which interact with one another in a cell are identified. In some embodiments, wherein at least 70%, at least 80%, at least 90% or more than 90% of the direct RNA-RNA interactions in the cell are identified. In some embodiments, the identification of the RNAs which interact with one another in a cell comprises performing sequence reads on said chimeric RNAs using an automated sequencing device. In some embodiments, said identification of the RNAs which interact with one another in a cell comprises identifying the chimeric sequences from all the sequence reads. In some embodiments, the method further comprises transforming the chimeric RNAs into annotated RNA clusters using a computer. In some embodiments, the method further comprises identifying direct interactions among said RNA clusters using a statistical test performed by a computer.

In some embodiments, an isolated complex is provided. The isolated complex can comprise a chimeric RNA cross-linked to a protein, wherein said chimeric RNA comprises RNAs which interact with one another in a cell. An isolated complex can also comprise a complex comprising a protein and nucleic acid, intermediate proteins and nucleic acid or a protein complex and nucleic acid, wherein the nucleic acid is RNA. In some embodiments, an isolated complex comprises a complex comprising a protein and nucleic acid, intermediate proteins and nucleic acid or a protein complex and nucleic acid, wherein the nucleic acid is RNA.

In some embodiments, a method for identifying a candidate therapeutic agent is provided, wherein the method comprises identifying RNAs which interact with one another in a cell using the method of any of the embodiments described herein and evaluating the ability of an agent to reduce or increase the interaction of said RNAs, wherein said agent is a candidate therapeutic agent if said agent is able to reduce or increase said interaction of said RNAs. In some embodiments the method for identifying RNAs which interact with one another in a cell comprises cross-linking RNA to protein and ligating RNAs cross-linked to the same protein molecule together to form a chimeric RNA. In some embodiments, said cross-linking of RNA to protein is performed on an intact cell or in a cell lysate. In some embodiments, said cross-linking comprises UV cross-linking. In some embodiments, the method further comprises associating said protein with an agent which facilitates immobilization of said protein on a surface. In some embodiments, said agent which facilitates immobilization comprises biotin. In some embodiments, the method further comprises fragmenting said RNAs cross-linked to the same protein molecule. In some embodiments, said fragmenting comprises contacting said RNAs cross-linked to the same protein molecule with an RNAse under conditions which facilitate partial digestion of said RNAs. In some embodiments, the method further comprises linking said RNAs cross-linked to the same protein molecule to an agent which facilitates recovery of said RNAs. In some embodiments, said linking comprises ligating the ends of said RNAs to said agent. In some embodiments, said agent which facilitates recovery of said RNAs comprises a nucleic acid. In some embodiments, said nucleic acid comprises a nucleic acid having biotin thereon. In some embodiments, said linking of said nucleic acid having biotin thereon to said ends of said RNAs comprises ligating said nucleic acid having biotin thereon to the 5′ ends of said RNAs prior to ligating said RNAs cross-linked to the same protein molecule together to form a chimeric RNA. In some embodiments, the method further comprises removing said biotin from the 5′ region of said chimeric RNA. In some embodiments, the method further comprises recovering said chimeric RNAs. In some embodiments, the method further comprises fragmenting said chimeric RNAs. In some embodiments, said fragmenting of said chimeric RNAs comprises contacting said chimeric RNAs with an RNAse under conditions which facilitate partial digestion of said RNAs. In some embodiments, the method further comprises reverse transcribing said chimeric RNAs to generate a chimeric cDNA. In some embodiments, the method further comprises determining at least a portion of the sequences in said chimeric RNAs or chimeric cDNAs which originate from each of the RNAs in said chimeric RNAs or chimeric cDNAs. In some embodiments, the method further comprises identifying the RNAs present in said chimeric RNAs, thereby identifying RNAs which interact with one another in a cell. In some embodiments, at least 100, at least 500, at least 1000 or more than 1000 RNA-RNA interactions in the cell are identified. In some embodiments, substantially all of the RNAs which interact with one another in a cell are identified. In some embodiments, wherein at least 70%, at least 80%, at least 90% or more than 90% of the direct RNA-RNA interactions in the cell are identified. In some embodiments, the identification of the RNAs which interact with one another in a cell comprises performing sequence reads on said chimeric RNAs using an automated sequencing device. In some embodiments, said identification of the RNAs which interact with one another in a cell comprises identifying the chimeric sequences from all the sequence reads. In some embodiments, the method further comprises transforming the chimeric RNAs into annotated RNA clusters using a computer. In some embodiments, the method further comprises identifying direct interactions among said RNA clusters using a statistical test performed by a computer. In some embodiments, said agent comprises a nucleic acid. In some embodiments, said agent comprises a chemical compound.

In some embodiments, a method of making a pharmaceutical is provided, wherein the method comprises formulating an agent identified using the method of any of the embodiments described herein, in a pharmaceutically acceptable carrier. In some embodiments, formulating an agent identified is performed by a method for identifying a candidate therapeutic agent, wherein the method comprises identifying RNAs which interact with one another in a cell using the method of any of the embodiments described herein and evaluating the ability of an agent to reduce or increase the interaction of said RNAs, wherein said agent is a candidate therapeutic agent if said agent is able to reduce or increase said interaction of said RNAs. In some embodiments the method for identifying RNAs which interact with one another in a cell comprises cross-linking RNA to protein and ligating RNAs cross-linked to the same protein molecule together to form a chimeric RNA. In some embodiments, said cross-linking of RNA to protein is performed on an intact cell or in a cell lysate. In some embodiments, said cross-linking comprises UV cross-linking. In some embodiments, the method further comprises associating said protein with an agent which facilitates immobilization of said protein on a surface. In some embodiments, said agent which facilitates immobilization comprises biotin. In some embodiments, the method further comprises fragmenting said RNAs cross-linked to the same protein molecule. In some embodiments, said fragmenting comprises contacting said RNAs cross-linked to the same protein molecule with an RNAse under conditions which facilitate partial digestion of said RNAs. In some embodiments, the method further comprises linking said RNAs cross-linked to the same protein molecule to an agent which facilitates recovery of said RNAs. In some embodiments, said linking comprises ligating the ends of said RNAs to said agent. In some embodiments, said agent which facilitates recovery of said RNAs comprises a nucleic acid. In some embodiments, said nucleic acid comprises a nucleic acid having biotin thereon. In some embodiments, said linking of said nucleic acid having biotin thereon to said ends of said RNAs comprises ligating said nucleic acid having biotin thereon to the 5′ ends of said RNAs prior to ligating said RNAs cross-linked to the same protein molecule together to form a chimeric RNA. In some embodiments, the method further comprises removing said biotin from the 5′ region of said chimeric RNA. In some embodiments, the method further comprises recovering said chimeric RNAs. In some embodiments, the method further comprises fragmenting said chimeric RNAs. In some embodiments, said fragmenting of said chimeric RNAs comprises contacting said chimeric RNAs with an RNAse under conditions which facilitate partial digestion of said RNAs. In some embodiments, the method further comprises reverse transcribing said chimeric RNAs to generate a chimeric cDNA. In some embodiments, the method further comprises determining at least a portion of the sequences in said chimeric RNAs or chimeric cDNAs which originate from each of the RNAs in said chimeric RNAs or chimeric cDNAs. In some embodiments, the method further comprises identifying the RNAs present in said chimeric RNAs, thereby identifying RNAs which interact with one another in a cell. In some embodiments, at least 100, at least 500, at least 1000 or more than 1000 RNA-RNA interactions in the cell are identified. In some embodiments, substantially all of the RNAs which interact with one another in a cell are identified. In some embodiments, wherein at least 70%, at least 80%, at least 90% or more than 90% of the direct RNA-RNA interactions in the cell are identified. In some embodiments, the identification of the RNAs which interact with one another in a cell comprises performing sequence reads on said chimeric RNAs using an automated sequencing device. In some embodiments, said identification of the RNAs which interact with one another in a cell comprises identifying the chimeric sequences from all the sequence reads. In some embodiments, the method further comprises transforming the chimeric RNAs into annotated RNA clusters using a computer. In some embodiments, the method further comprises identifying direct interactions among said RNA clusters using a statistical test performed by a computer. In some embodiments, said agent comprises a nucleic acid. In some embodiments, said agent comprises a chemical compound.

In some embodiments a pharmaceutical is provided, wherein the pharmaceutical is made using the method of any of the embodiments described herein. In some embodiments, the method comprises formulating an agent identified using the method of any of the embodiments described herein, in a pharmaceutically acceptable carrier. In some embodiments, formulating an agent identified is performed by a method for identifying a candidate therapeutic agent, wherein the method comprises identifying RNAs which interact with one another in a cell using the method of any of the embodiments described herein and evaluating the ability of an agent to reduce or increase the interaction of said RNAs, wherein said agent is a candidate therapeutic agent if said agent is able to reduce or increase said interaction of said RNAs. In some embodiments the method for identifying RNAs which interact with one another in a cell comprises cross-linking RNA to protein and ligating RNAs cross-linked to the same protein molecule together to form a chimeric RNA. In some embodiments, said cross-linking of RNA to protein is performed on an intact cell or in a cell lysate. In some embodiments, said cross-linking comprises UV cross-linking. In some embodiments, the method further comprises associating said protein with an agent which facilitates immobilization of said protein on a surface. In some embodiments, said agent which facilitates immobilization comprises biotin. In some embodiments, the method further comprises fragmenting said RNAs cross-linked to the same protein molecule. In some embodiments, said fragmenting comprises contacting said RNAs cross-linked to the same protein molecule with an RNAse under conditions which facilitate partial digestion of said RNAs. In some embodiments, the method further comprises linking said RNAs cross-linked to the same protein molecule to an agent which facilitates recovery of said RNAs. In some embodiments, said linking comprises ligating the ends of said RNAs to said agent. In some embodiments, said agent which facilitates recovery of said RNAs comprises a nucleic acid. In some embodiments, said nucleic acid comprises a nucleic acid having biotin thereon. In some embodiments, said linking of said nucleic acid having biotin thereon to said ends of said RNAs comprises ligating said nucleic acid having biotin thereon to the 5′ ends of said RNAs prior to ligating said RNAs cross-linked to the same protein molecule together to form a chimeric RNA. In some embodiments, the method further comprises removing said biotin from the 5′ region of said chimeric RNA. In some embodiments, the method further comprises recovering said chimeric RNAs. In some embodiments, the method further comprises fragmenting said chimeric RNAs. In some embodiments, said fragmenting of said chimeric RNAs comprises contacting said chimeric RNAs with an RNAse under conditions which facilitate partial digestion of said RNAs. In some embodiments, the method further comprises reverse transcribing said chimeric RNAs to generate a chimeric cDNA. In some embodiments, the method further comprises determining at least a portion of the sequences in said chimeric RNAs or chimeric cDNAs which originate from each of the RNAs in said chimeric RNAs or chimeric cDNAs. In some embodiments, the method further comprises identifying the RNAs present in said chimeric RNAs, thereby identifying RNAs which interact with one another in a cell. In some embodiments, at least 100, at least 500, at least 1000 or more than 1000 RNA-RNA interactions in the cell are identified. In some embodiments, substantially all of the RNAs which interact with one another in a cell are identified. In some embodiments, wherein at least 70%, at least 80%, at least 90% or more than 90% of the direct RNA-RNA interactions in the cell are identified. In some embodiments, the identification of the RNAs which interact with one another in a cell comprises performing sequence reads on said chimeric RNAs using an automated sequencing device. In some embodiments, said identification of the RNAs which interact with one another in a cell comprises identifying the chimeric sequences from all the sequence reads. In some embodiments, the method further comprises transforming the chimeric RNAs into annotated RNA clusters using a computer. In some embodiments, the method further comprises identifying direct interactions among said RNA clusters using a statistical test performed by a computer. In some embodiments, said agent comprises a nucleic acid. In some embodiments, said agent comprises a chemical compound.

In some embodiments, a method for generating chimeric RNAs comprising RNAs which interact with one another in a cell is provided, wherein the method comprises cross-linking RNA to protein intermediates and/or a protein complex and ligating RNAs cross-linked to protein intermediates and/or the protein complex together to form a chimeric RNA, and wherein the protein complex comprises two or more interacting proteins. In some embodiments, said cross-linking of RNA to the protein intermediates and/or the protein complex is performed on an intact cell or in a cell lysate. In some embodiments, said cross-linking comprises UV cross-linking. In some embodiments, the method further comprises associating said protein intermediates and/or the protein complex with an agent which facilitates immobilization of said protein intermediates and/or the protein complex on a surface. In some embodiments, said agent which facilitates immobilization comprises biotin. In some embodiments, the method further comprises fragmenting said RNAs cross-linked to the at least one protein molecule. In some embodiments, fragmenting comprises contacting said RNAs cross-linked to the protein intermediates and/or the protein complex with an RNAse under conditions which facilitate partial digestion of said RNAs. In some embodiments, the method further comprises linking said RNAs cross-linked to the protein intermediates and/or the protein complex to an agent which facilitates recovery of said RNAs. In some embodiments, said linking comprises ligating the ends of said RNAs to said agent. In some embodiments, said agent which facilitates recovery of said RNAs comprises a nucleic acid. In some embodiments, said nucleic acid comprises a nucleic acid having biotin thereon. In some embodiments, said linking of said nucleic acid having biotin thereon to said ends of said RNAs comprises ligating said nucleic acid having biotin thereon to the 5′ ends of said RNAs prior to ligating said RNAs cross-linked to the protein intermediates and/or the protein complex together to form a chimeric RNA. In some embodiments, the method further comprises removing said biotin from the 5′ region of said chimeric RNA. In some embodiments, the method further comprises recovering said chimeric RNAs. In some embodiments, the method further comprises fragmenting said chimeric RNAs. In some embodiments, said fragmenting of said chimeric RNAs comprises contacting said chimeric RNAs with an RNAse under conditions which facilitate partial digestion of said RNAs. In some embodiments, the method further comprises reverse transcribing said chimeric RNAs to generate a chimeric cDNA. In some embodiments, the method further comprises identifying the RNAs present in said chimeric RNAs, thereby identifying RNAs which interact with one another in a cell. In some embodiments, at least 100, at least 500, at least 1000 or more than 1000 RNA-RNA interactions in the cell are identified. In some embodiments, substantially all of the RNAs which interact with one another in a cell are identified. In some embodiments, at least 70%, at least 80%, at least 90% or more than 90% of the direct RNA-RNA interactions in the cell are identified. In some embodiments, the identification of the RNAs which interact with one another in a cell comprises performing sequence reads on said chimeric RNAs using an automated sequencing device. In some embodiments, said identification of the RNAs which interact with one another in a cell comprises identifying the chimeric sequences from all the sequence reads. In some embodiments, the method further comprises transforming the chimeric RNAs into annotated RNA clusters using a computer. In some embodiments, the method further comprises identifying direct interactions among said RNA clusters using a statistical test performed by a computer. In some embodiments, said RNAs which interact with each other in the cell are cross-linked to different proteins in said protein intermediate or protein complex.

In some embodiments, an isolated complex comprising a chimeric RNA cross-linked to protein intermediates and/or a protein complex is provided, wherein said chimeric RNA comprises RNAs which interact with one another in a cell, wherein the protein complex comprises two or more interacting proteins. In some embodiments, said chimeric RNA comprises RNAs which are cross-linked to different proteins in said protein intermediate or protein complex.

Each reference listed herein is incorporated herein by reference in its entirety.

### REFERENCES

1. Engreitz, J. M. et al. RNA-RNA interactions enable specific targeting of noncoding RNAs to nascent Pre-mRNAs and chromatin sites. Cell 159, 188-199, doi:10.1016/j.cell.2014.08.018 (2014).
2. Ray, D. et al. A compendium of RNA-binding motifs for decoding gene regulation. Nature 499, 172-177, doi:10.1038/nature12311 (2013).
3. Meister, G. Argonaute proteins: functional insights and emerging roles. Nat Rev Genet 14, 447-459, doi:10.1038/nrg3462 (2013).
4. Hafner, M. et al. Transcriptome-wide identification of RNA-binding protein and microRNA target sites by PAR-CLIP. Cell 141, 129-141, doi:10.1016/j.cell.2010.03.009 (2010).
5. Granneman, S., Kudla, G., Petfalski, E. & Tollervey, D. Identification of protein binding sites on U3 snoRNA and pre-rRNA by UV cross-linking and high-throughput analysis of cDNAs. Proceedings of the National Academy of Sciences of the United States of America 106, 9613-9618, doi:10.1073/pnas.0901997106 (2009).
6. Chi, S. W., Zang, J. B., Mele, A. & Darnell, R. B. Argonaute HITS-CLIP decodes microRNA-mRNA interaction maps. Nature 460, 479-486, doi:10.1038/nature08170 (2009).
7. Helwak, A., Kudla, G., Dudnakova, T. & Tollervey, D. Mapping the human miRNA interactome by CLASH reveals frequent noncanonical binding. Cell 153, 654-665, doi:10.1016/j.cell.2013.03.043 (2013).
8. Kudla, G., Granneman, S., Hahn, D., Beggs, J. D. & Tollervey, D. Cross-linking, ligation, and sequencing of hybrids reveals RNA-RNA interactions in yeast. Proc Natl Acad Sci USA 108, 10010-10015, doi:10.1073/pnas.1017386108 (2011).
9. Nicolas, F. E. Experimental validation of microRNA targets using a luciferase reporter system. Methods in molecular biology 732, 139-152, doi:10.1007/978-1-61779-083-6_11 (2011).
10. Lal, A. et al. Capture of microRNA-bound mRNAs identifies the tumor suppressor miR-34a as a regulator of growth factor signaling. PLoS Genet 7, e1002363, doi:10.1371/journal.pgen.1002363 (2011).
11. Du, T. & Zamore, P. D. Beginning to understand microRNA function. Cell Res 17, 661-663, doi:10.1038/cr.2007.67 (2007).
12. Kalhor, R., Tjong, H., Jayathilaka, N., Alber, F. & Chen, L. Genome architectures revealed by tethered chromosome conformation capture and population-based modeling. Nature biotechnology 30, 90-98, doi:10.1038/nbt.2057 (2012).
13. Belton, J. M. et al. Hi-C: a comprehensive technique to capture the conformation of genomes. Methods 58, 268-276, doi:10.1016/j.ymeth.2012.05.001 (2012).
14. Baigude, H., Ahsanullah, Li, Z., Zhou, Y. & Rana, T. M. miR-TRAP: a benchtop chemical biology strategy to identify microRNA targets. Angew Chem Int Ed Engl 51, 5880-5883, doi:10.1002/anie.201201512 (2012).
15. Loeb, G. B. et al. Transcriptome-wide miR-155 binding map reveals widespread noncanonical microRNA targeting. Mol Cell 48, 760-770, doi:10.1016/j.molcel.2012.10.002 (2012).
16. Wang, Z. et al. iCLIP predicts the dual splicing effects of TIA-RNA interactions. PLoS Biol 8, e1000530, doi:10.1371/journal.pbio.1000530 (2010).
17. Konig, J. et al. iCLIP reveals the function of hnRNP particles in splicing at individual nucleotide resolution. Nat Struct Mol Biol 17, 909-915, doi:10.1038/nsmb.1838 (2010).
18. Nowak, D. E., Tian, B. & Brasier, A. R. Two-step cross-linking method for identification of NF-kappaB gene network by chromatin immunoprecipitation. Biotechniques 39, 715-725 (2005).
19. Zeng, P. Y., Vakoc, C. R., Chen, Z. C., Blobel, G. A. & Berger, S. L. In vivo dual cross-linking for identification of indirect DNA-associated proteins by chromatin immunoprecipitation. BioTechniques 41, 694-698 (2006).
20. Zhao, J. et al. Genome-wide identification of polycomb-associated RNAs by RIP-seq. Mol Cell 40, 939-953, doi:10.1016/j.molcel.2010.12.011 (2010).
21. Yu, P. et al. Spatiotemporal clustering of the epigenome reveals rules of dynamic gene regulation. Genome Res 23, 352-364, doi:10.1101/gr.144949.112 (2013).
22. Ender, C. et al. A human snoRNA with microRNA-like functions. Mol Cell 32, 519-528, doi:10.1016/j.molcel.2008.10.017 (2008).
23. Brameier, M., Herwig, A., Reinhardt, R., Walter, L. & Gruber, J. Human box C/D snoRNAs with miRNA like functions: expanding the range of regulatory RNAs. Nucleic Acids Res 39, 675-686, doi:10.1093/nar/gkq776 (2011).
24. Guttman, M. et al. Chromatin signature reveals over a thousand highly conserved large non-coding RNAs in mammals. Nature 458, 223-227, doi:10.1038/nature07672 (2009).
25. Barabasi, A. L. & Oltvai, Z. N. Network biology: understanding the cell's functional organization. Nat Rev Genet 5, 101-113, doi:10.1038/nrg1272 (2004).
26. Shalgi, R., Pilpel, Y. & Oren, M. Repression of transposable-elements—a microRNA anti-cancer defense mechanism? Trends in genetics: TIG 26, 253-259, doi:10.1016/j.tig.2010.03.006 (2010).
27. Yuan, Z., Sun, X., Liu, H. & Xie, J. MicroRNA genes derived from repetitive elements and expanded by segmental duplication events in mammalian genomes. PloS one 6, e17666, doi:10.1371/journal.pone.0017666 (2011).
28. Schwartz, S. et al. Transcriptome-wide mapping reveals widespread dynamic-regulated pseudouridylation of ncRNA and mRNA. Cell 159, 148-162, doi:10.1016/j.cell.2014.08.028 (2014).
29. Bellaousov, S., Reuter, J. S., Seetin, M. G. & Mathews, D. H. RNAstructure: web servers for RNA secondary structure prediction and analysis. Nucleic Acids Research 41, W471-W474, doi:Doi 10.1093/Nar/Gkt290 (2013).
30. Gong, C. & Maquat, L. E. lncRNAs transactivate STAU1-mediated mRNA decay by duplexing with 3′ UTRs via Alu elements. Nature 470, 284-288, doi:10.1038/nature09701 (2011).
31. Cooper, G. M. et al. Distribution and intensity of constraint in mammalian genomic sequence. Genome Res 15, 901-913, doi:10.1101/gr.3577405 (2005).
32. Kiss, T., Fayet-Lebaron, E. & Jady, B. E. Box H/ACA small ribonucleoproteins. Mol Cell 37, 597-606, doi:10.1016/j.molcel.2010.01.032 (2010).

1. A method for generating chimeric RNAs comprising RNAs which interact with one another in a cell comprising cross-linking RNA to protein and ligating RNAs cross-linked to the same protein molecule together to form a chimeric RNA.

2. The method of claim 1, wherein said cross-linking of RNA to protein is performed on an intact cell or in a cell lysate.

3. The method of claim 1, wherein said cross-linking comprises UV cross-linking.

4. The method of claim 1, further comprising associating said protein with an agent which facilitates immobilization of said protein on a surface.

5. (canceled)

6. The method of claim 1, further comprising fragmenting said RNAs cross-linked to the same protein molecule.

7. (canceled)

8. The method of claim 1, further comprising linking said RNAs cross-linked to the same protein molecule to an agent which facilitates recovery of said RNAs.

9. (canceled)

10. (canceled)

11. (canceled)

12. (canceled)

13. (canceled)

14. The method of claim 1, further comprising recovering said chimeric RNAs.

15. The method of claim 1, further comprising fragmenting said chimeric RNAs.

16. (canceled)

17. The method of claim 1, further comprising reverse transcribing said chimeric RNAs to generate a chimeric cDNA.

18. The method of claim 1, further comprising determining at least a portion of the sequences in said chimeric RNAs or chimeric cDNAs which originate from each of the RNAs in said chimeric RNAs or chimeric cDNAs.

19. The method of claim 1, further comprising identifying the RNAs present in said chimeric RNAs, thereby identifying RNAs which interact with one another in a cell.

20. The method of claim 19, wherein at least 100, at least 500, at least 1000 or more than 1000 RNA-RNA interactions in the cell are identified.

21. The method of claim 19, wherein substantially all of the RNAs which interact with one another in a cell are identified.

22. The method of claim 21, wherein at least 70%, at least 80%, at least 90% or more than 90% of the direct RNA-RNA interactions in the cell are identified.

23. (canceled)

24. (canceled)

25. The method of claim 19, further comprising transforming the chimeric RNAs into annotated RNA clusters using a computer.

26. The method of claim 25, further comprising identifying direct interactions among said RNA clusters using a statistical test performed by a computer.

27. An isolated complex comprising a chimeric RNA cross-linked to a protein, wherein said chimeric RNA comprises RNAs which interact with one another in a cell.

28. A method for identifying a candidate therapeutic agent comprising:
identifying RNAs which interact with one another in a cell using the method of claim 1; and
evaluating the ability of an agent to reduce or increase the interaction of said RNAs, wherein said agent is a candidate therapeutic agent if said agent is able to reduce or increase said interaction of said RNAs.

29. (canceled)

30. (canceled)

31. (canceled)

32. (canceled)

33. A method for generating chimeric RNAs comprising RNAs which interact with one another in a cell comprising cross-linking RNA to protein intermediates and/or a protein complex and ligating RNAs cross-linked to protein intermediates and/or the protein complex together to form a chimeric RNA, and wherein the protein complex comprises two or more interacting proteins.

34. The method of claim 33, wherein said cross-linking of RNA to the protein intermediates and/or the protein complex is performed on an intact cell or in a cell lysate.

35. The method of claim 33 wherein said cross-linking comprises UV cross-linking.

36. The method of claim 33, further comprising associating said protein intermediates and/or the protein complex with an agent which facilitates immobilization of said protein intermediates and/or the protein complex on a surface.

37. (canceled)

38. The method of claim 33, further comprising fragmenting said RNAs cross-linked to the at least one protein molecule.

39. (canceled)

40. The method of claim 33, further comprising linking said RNAs cross-linked to the protein intermediates and/or the protein complex to an agent which facilitates recovery of said RNAs.

41. (canceled)

42. (canceled)

43. (canceled)

44. (canceled)

45. (canceled)

46. The method of claim 33, further comprising recovering said chimeric RNAs.

47. The method of claim 33, further comprising fragmenting said chimeric RNAs.

48. (canceled)

49. The method of claim 33, further comprising reverse transcribing said chimeric RNAs to generate a chimeric cDNA.

50. The method of claim 33, further comprising determining at least a portion of the sequences in said chimeric RNAs or chimeric cDNAs which originate from each of the RNAs in said chimeric RNAs or chimeric cDNAs.

51. The method of claim 33, further comprising identifying the RNAs present in said chimeric RNAs, thereby identifying RNAs which interact with one another in a cell.

52. The method of claim 51, wherein at least 100, at least 500, at least 1000 or more than 1000 RNA-RNA interactions in the cell are identified.

53. The method of claim 51, wherein substantially all of the RNAs which interact with one another in a cell are identified.

54. The method of claim 53, wherein at least 70%, at least 80%, at least 90% or more than 90% of the direct RNA-RNA interactions in the cell are identified.

55. (canceled)

56. (canceled)

57. The method of claim 51, further comprising transforming the chimeric RNAs into annotated RNA clusters using a computer.

58. The method of claim 57, further comprising identifying direct interactions among said RNA clusters using a statistical test performed by a computer.

59. The method of claim 33, wherein said RNAs which interact with each other in the cell are cross-linked to different proteins in said protein intermediate or protein complex.

60. An isolated complex comprising a chimeric RNA cross-linked to protein intermediates and/or a protein complex, wherein said chimeric RNA comprises RNAs which interact with one another in a cell, wherein the protein complex comprises two or more interacting proteins.

61. (canceled)


SEQUENCE LISTING (21 sequences)

SEQ ID NO: 1 - RNA, 24 nt, Artificial Sequence
  misc_feature (5)...(5): biotinylated
  Biotin-tagged RNA linker
cuagtagcccaugcaaugcgagga

SEQ ID NO: 2 - DNA, 24 bp, Artificial Sequence
  complementary DNA oligonucleotide
  misc_feature (1)...(5): phosphorothioate bond between nucleic acids
tcgcattgcatgggctactagcat

SEQ ID NO: 3 - DNA, 20 bp, Artificial Sequence
  3′ reverse transcription (RT) adapter
  misc_feature (1)...(1): adaptor 4rApp
  misc_feature (20)...(20): adaptor 3ddC
agatcggaagagcggttcag

SEQ ID NO: 4 - DNA, 53 bp, Artificial Sequence
  RT primer
  misc_feature (1)...(1): 5phos
  misc_feature (1)...(2): N is A, C, T or G
  misc_feature (3)...(3): N is A or C
  misc_feature (4)...(4): N is G or A
  misc_feature (5)...(5): N is G, C or T
  misc_feature (6)...(6): N is T or C
  misc_feature (7)...(10): N is A, C, T or G
nnnnnnnnnnagatcggaagagcgtcgtggatcctgaaccgctcttccgatct

SEQ ID NO: 5 - DNA, 10 bp, Artificial Sequence
  Reverse complement
  misc_feature (1)...(1): N is A, C, T or G
  misc_feature (2)...(2): N is A, C, T or G
  misc_feature (3)...(3): N is A, C, T or G
  misc_feature (4)...(4): N is A, C, T or G
  misc_feature (5)...(5): N is A or C
  misc_feature (6)...(6): N is G or A
  misc_feature (7)...(7): N is G, C or T
  misc_feature (8)...(8): N is T or C
  misc_feature (9)...(9): N is A, C, T or G
  misc_feature (10)...(10): N is A, C, T or G
nnnnnnnnnn

SEQ ID NO: 6 - DNA, 58 bp, Artificial Sequence
  Illumina PE PCR Forward Primer 1.0
aatgatacggcgaccaccgagatctacactctttccctacacgacgctcttccgatct

SEQ ID NO: 7 - DNA, 61 bp, Artificial Sequence
  PE PCR Reverse Primer 2.0
caagcagaagacggcatacgagatcggtctcggcattcctgctgaaccgctcttccgatct

SEQ ID NO: 8 - DNA, 28 bp, Artificial Sequence
  Cut_oligo
  modified_base (28)...(28): idT
gttcaggatccacgacgctcttcaaaat

SEQ ID NO: 9 - DNA, 19 bp, Artificial Sequence
  forward primer DP5
cacgacgctcttccgatct

SEQ ID NO: 10 - DNA, 20 bp, Artificial Sequence
  reverse primer DP3
ctgaaccgctcttccgatct

SEQ ID NO: 11 - RNA, 24 nt, Artificial Sequence
  Biotinylated RNA linker
  misc_feature (5)...(5): biotinylated
cuagtagcccaugcaaugcgagga

SEQ ID NO: 12 - DNA, 24 bp, Artificial Sequence
  Complementary DNA strand with RNA linker
  misc_feature (1)...(5): phosphorothioate bond between the nucleic acid bases
tcgcattgcatgggctactagcat

SEQ ID NO: 13 - DNA, 20 bp, Artificial Sequence
  Pre-adenylated RT adapter (RNase-free HPLC-purified from IDT)
  misc_feature (1)...(1): 54rApp
  misc_feature (20)...(20): 3ddC
agatcggaagagcggttcag

SEQ ID NO: 14 - DNA, 52 bp, Artificial Sequence
  RT Primer for the ES-1 sample
  misc_feature (1)...(1): 5Phos
  misc_feature (1)...(1): N is A, C, T or G
  misc_feature (2)...(2): N is A, C, T or G
  misc_feature (7)...(7): N is A, C, T or G
  misc_feature (8)...(8): N is A, C, T or G
  misc_feature (9)...(9): N is A, C, T or G
nnaggtnnnagatcggaagagcgtcgtggatcctgaaccgctcttccgatct

SEQ ID NO: 15 - DNA, 53 bp, Artificial Sequence
  RT Primer for the ES-2 and MEF samples
  misc_feature (1)...(1): 5Phos
  misc_feature (1)...(1): N is A, C, T or G
  misc_feature (2)...(2): N is A, C, T or G
  misc_feature (7)...(7): N is A, C, T or G
  misc_feature (8)...(8): N is A, C, T or G
  misc_feature (9)...(9): N is A, C, T or G
  misc_feature (10)...(10): N is A, C, T or G
nncgccnnnnagatcggaagagcgtcgtggatcctgaaccgctcttccgatct

SEQ ID NO: 16 - DNA, 53 bp, Artificial Sequence
  RT Primer for the ES-indirect sample
  misc_feature (1)...(1): 5Phos
  misc_feature (1)...(1): N is A, C, T or G
  misc_feature (2)...(2): N is A, C, T or G
  misc_feature (7)...(7): N is A, C, T or G
  misc_feature (8)...(8): N is A, C, T or G
  misc_feature (9)...(9): N is A, C, T or G
  misc_feature (10)...(10): N is A, C, T or G
nncattnnnnagatcggaagagcgtcgtggatcctgaaccgctcttccgatct

SEQ ID NO: 17 - DNA, 28 bp, Artificial Sequence
  Cut_oligo (HPLC-purified from IDT)
  misc_feature (28)...(28): idT
gttcaggatccacgacgctcttcaaaat

SEQ ID NO: 18 - DNA, 19 bp, Artificial Sequence
  Truncated PCR Forward Primer DP5
cacgacgctcttccgatct

SEQ ID NO: 19 - DNA, 20 bp, Artificial Sequence
  Truncated PCR Reverse Primer DP3
ctgaaccgctcttccgatct

SEQ ID NO: 20 - DNA, 58 bp, Artificial Sequence; identical to SEQ ID NO: 6
  Illumina PE PCR Forward Primer 1.0

SEQ ID NO: 21 - DNA, 61 bp, Artificial Sequence; identical to SEQ ID NO: 7
  Illumina PE PCR Reverse Primer 2.0